(19) United States
(12) Patent Application Publication
(10) Pub. No.: US 2024/0174998 A1
(43) Pub. Date: May 30, 2024

(54) MODIFIED CASCADE RIBONUCLEOPROTEINS AND USES THEREOF

(71) Applicant: Caribou Biosciences, Inc., Berkeley, CA (US)

(72) Inventors: Stan Johan Josef Brouns, Wageningen (NL); John van der Oast, Renkum (NL)

(73) Assignee: Caribou Biosciences, Inc., Berkeley, CA (US)

(21) Appl. No.: 18/422,843

(22) Filed: Jan. 25, 2024

Related U.S. Application Data
(63) Continuation of application No. 17/179,215, filed on Feb. 18, 2021, now Pat. No. 11,939,604.
Application No. 17/179,215 is a continuation of application No. 16/914,203, filed on Jun. 26, 2020, now Pat. No. 10,954,498.
Application No. 16/914,203 is a continuation of application No. 16/554,225, filed on Aug. 28, 2019, now Pat. No. 10,711,257.
Application No. 16/554,225 is a continuation of application No. 15/802,413, filed on Nov. 2, 2017, now Pat. No. 10,435,678.
Application No. 15/802,413 is a continuation of application No. 14/997,474, filed on Jan. 15, 2016, now Pat. No. 9,885,026.
Application No. 14/997,474 is a continuation of application No. 14/326,099, filed on Jul. 8, 2014, now abandoned.
Application No. 14/326,099 is a continuation of application No. 14/240,735, filed on Feb. 24, 2014, now abandoned.

(51) Int. Cl. C12N 9/22; A61K 48/00; C07K 14/245; C07K 14/47; C12N 9/16; C12N 15/62; C12N 15/66; C12N 15/70; C12N 15/74; C12N 15/81; C12N 15/82; C12N 15/86; C12N 15/90; A61K 38/00

(52) U.S. Cl. CPC C12N 9/22; A61K 48/005; C07K 14/245; C07K 14/47; C12N 9/16; C12N 15/62; C12N 15/66; C12N 15/70; C12N 15/74; C12N 15/81; C12N 15/82; C12N 15/86; C12N 15/902; C12N 15/907; A61K 38/00; C07K 2319/09; C07K 2319/22; C07K 2319/60; C07K 2319/71; C07K 2319/80; C07K 2319/85; C12N 2310/20; C12Y 301/21004

(57) ABSTRACT

A clustered regularly interspaced short palindromic repeat (CRISPR)-associated complex for adaptive antiviral defence (Cascade); the Cascade protein complex comprising at least CRISPR-associated protein subunits Cas7, Cas5 and Cas6 which includes at least one subunit with an additional amino acid sequence possessing nucleic acid or chromatin modifying, visualising, transcription activating or transcription repressing activity. The Cascade complex with additional activity is combined with an RNA molecule to produce a ribonucleoprotein complex. The RNA molecule is selected to have substantial complementarity to a target sequence. Targeted ribonucleoproteins can be used as genetic engineering tools for precise cutting of nucleic acids in homologous recombination, non-homologous end joining, gene modification, gene integration, mutation repair or for their visualisation, transcriptional activation or repression. A pair of ribonucleotides fused to FokI dimers may be used to generate double-strand breakages in the DNA to facilitate these applications in a sequence-specific manner.

This application is a Continuation of U.S. patent application Ser. No. 16/914,203, filed 26 Jun. 2020, now allowed, which is a Continuation of U.S. patent application Ser. No. 16/554,225, filed 28 Aug. 2019, now U.S. Pat. No. 10,711,257, issued 14 Jul. 2020, which is a Continuation of U.S. patent application Ser. No. 15/802,413, filed 2 Nov. 2017, now U.S. Pat. No. 10,435,678, issued 8 Oct. 2019, which is a Continuation of U.S. patent application Ser. No. 14/997,474, filed 15 Jan. 2016, now U.S. Pat. No. 9,885,026, issued 6 Feb. 2018, which is a Continuation of U.S. patent application Ser. No. 14/326,099, filed 8 Jul. 2014, now abandoned, which is a Continuation of U.S. patent application Ser. No. 14/240,735, filed 24 Feb. 2014, now abandoned, which is a National Stage Entry of PCT/EP2012/076674, filed 21 Dec. 2012, now expired, which claims the benefit of priority under 35 U.S.C. 119(a)/(b) of United Kingdom Patent Application No. GB1122458.1, filed 30 Dec. 2011, now abandoned, the contents of all of which are each of which applications is incorporated herein by reference in its entirety their entireties. A certified copy of the foreign priority document (GB 1122458.1) is of record in U.S. patent application Ser. No. 14/240,735.

The invention relates to the field of genetic engineering and more particularly to the area of gene and/or genome modification of organisms, including prokaryotes and eukaryotes. The invention also concerns methods of making site specific tools for use in methods of genome analysis and genetic modification, whether in vivo or in vitro. The invention more particularly relates to the field of ribonucleoproteins which recognise and associate with nucleic acid sequences in a sequence specific way.

Bacteria and archaea have a wide variety of defense mechanisms against invasive DNA. So called CRISPR/Cas defense systems provide adaptive immunity by integrating plasmid and viral DNA fragments in loci of clustered regularly interspaced short palindromic repeats (CRISPR) on the host chromosome. The viral or plasmid-derived sequences, known as spacers, are separated from each other by repeating host-derived sequences. These repetitive elements are the genetic memory of this immune system and each CRISPR locus contains a diverse repertoire of unique ‘spacer’ sequences acquired during previous encounters with foreign genetic elements.

Acquisition of foreign DNA is the first step of immunization, but protection requires that the CRISPR is transcribed and that these long transcripts are processed into short CRISPR-derived RNAs (crRNAs) that each contains a unique spacer sequence complementary to a foreign nucleic acid challenger.

In addition to the crRNA, genetic experiments in several organisms have revealed that a unique set of CRISPR-associated (Cas) proteins is required for the steps of acquiring immunity, for crRNA biogenesis and for targeted interference. Also, a subset of Cas proteins from phylogenetically distinct CRISPR systems have been shown to assemble into large complexes that include a crRNA.

A recent re-evaluation of the diversity of CRISPR/Cas systems has resulted in a classification into three distinct types (Makarova K. et al (2011) Nature Reviews Microbiology—AOP 9 May 2011; doi: 10.1038/nrmicro2577) that vary in cas gene content, and display major differences throughout the CRISPR defense pathway. (The Makarova classification and nomenclature for CRISPR-associated genes is adopted in the present specification.) RNA transcripts of CRISPR loci (pre-crRNA) are cleaved specifically in the repeat sequences by CRISPR associated (Cas) endoribonucleases in type I and type III systems or by RNase III in type II systems; the generated crRNAs are utilized by a Cas protein complex as a guide RNA to detect complementary sequences of either invading DNA or RNA. Cleavage of target nucleic acids has been demonstrated in vitro for the Pyrococcus furiosus type III-B system, which cleaves RNA in a ruler-anchored mechanism, and, more recently, in vivo for the Streptococcus thermophiles type II system, which cleaves DNA in the complementary target sequence (protospacer). In contrast, for type I systems the mechanism of CRISPR-interference is still largely unknown.

The model organism Escherichia coli strain K12 possesses a CRISPR/Cas type I-E (previously known as CRISPR subtype E (Cse)). It contains eight cas genes (cas1, cas2, cas3 and cse1, cse2, cas7, cas5, cas6e) and a downstream CRISPR (type-2 repeats). In Escherichia coli K12 the eight cas genes are encoded upstream of the CRISPR locus. Cas1 and Cas2 do not appear to be needed for target interference, but are likely to participate in new target sequence acquisition. In contrast, six Cas proteins: Cse1, Cse2, Cas3, Cas7, Cas5 and Cas6e (previously also known as CasA, CasB, Cas3, CasC/Cse4, CasD and CasE/Cse3 respectively) are essential for protection against lambda phage challenge. Five of these proteins: Cse1, Cse2, Cas7, Cas5 and Cas6e (previously known as CasA, CasB, CasC/Cse4, CasD and CasE/Cse3 respectively) assemble with a crRNA to form a multi-subunit ribonucleoprotein (RNP) referred to as Cascade.

In E. coli, Cascade is a 405 kDa ribonucleoprotein complex composed of an unequal stoichiometry of five functionally essential Cas proteins: Cse1₁Cse2₂Cas7₆Cas5₁Cas6e₁ (i.e. under previous nomenclature CasA₁B₂C₆D₁E₁) and a 61-nt CRISPR-derived RNA. Cascade is an obligate RNP that relies on the crRNA for complex assembly and stability, and for the identification of invading nucleic acid sequences. Cascade is a surveillance complex that finds and binds foreign nucleic acids that are complementary to the spacer sequence of the crRNA.

Jore et al. (2011) entitled “Structural basis for CRISPR RNA-guided DNA recognition by Cascade” Nature Structural & Molecular Biology 18: 529-537 describes how there is a cleavage of the pre-crRNA transcript by the Cas6e subunit of Cascade, resulting in the mature 61 nt crRNA being retained by the CRISPR complex. The crRNA serves as a guide RNA for sequence specific binding of Cascade to double stranded (ds) DNA molecules through base pairing between the crRNA spacer and the complementary protospacer, forming a so-called R-loop. This is known to be an ATP-independent process.

Brouns S.J.J., et al (2008) entitled “Small CRISPR RNAs guide antiviral defense in prokaryotes” Science 321: 960-964 teaches that Cascade loaded with a crRNA requires Cas3 for in vivo phage resistance.

Marraffini L. & Sontheimer E. (2010) entitled “CRISPR interference: RNA-directed adaptive immunity in bacteria and archaea” Nature Reviews Genetics 11: 181-190 is a review article which summarises the state of knowledge in the art in the field. Some suggestions are made about CRISPR-based applications and technologies, but this is mainly in the area of generating phage resistant strains of domesticated bacteria for the dairy industry. The specific cleavage of RNA molecules in vitro by a crRNP complex in Pyrococcus furiosus is suggested as something which awaits further development. Manipulation of CRISPR systems is also suggested as a possible way of reducing transmission of antibiotic-resistant bacterial strains in hospitals. The authors stress that further research effort will be needed to explore the potential utility of the technology in these areas.

US2011236530 A1 (Manoury et al.) entitled “Genetic cluster of strains of Streptococcus thermophilus having unique rheological properties for dairy fermentation” discloses certain S. thermophilus strains which ferment milk so that it is highly viscous and weakly ropy. A specific CRISPR locus of defined sequence is disclosed.

US2011217739 A1 (Terns et al.) entitled “Cas6 polypeptides and methods of use” discloses polypeptides which have Cas6 endoribonuclease activity. The polypeptides cleave a target RNA polynucleotide having a Cas6 recognition domain and cleavage site. Cleavage may be carried out in vitro or in vivo. Microbes such as E. coli or Haloferax volcanii are genetically modified so as to express Cas6 endoribonuclease activity.

WO2010054154 (Danisco) entitled “Bifidobacteria CRISPR sequences” discloses various CRISPR sequences found in Bifidobacteria and their use in making genetically altered strains of the bacteria which are altered in their phage resistance characteristics.

US2011189776 A1 (Terns et al.) entitled “Prokaryotic RNAi-like system and methods of use” describes methods of inactivating target polynucleotides in vitro or in prokaryotic microbes in vivo. The methods use a psiRNA having a 5′ region of 5-10 nucleotides chosen from a repeat from a CRISPR locus immediately upstream of a spacer. The 3′ region is substantially complementary to a portion of the target polynucleotide. Also described are polypeptides having endonuclease activity in the presence of psiRNA and target polynucleotide.

EP2341149 A1 (Danisco) entitled “Use of CRISPR associated genes (CAS) describes how one or more Cas genes can be used for modulating resistance of bacterial cells against bacteriophage; particularly bacteria which provide a starter culture or probiotic culture in dairy products.

WO2010075424 (The Regents of the University of California) entitled “Compositions and methods for downregulating prokaryotic genes” discloses an isolated polynucleotide comprising a CRISPR array. At least one spacer of the CRISPR is complementary to a gene of a prokaryote so that is can down-regulate expression of the gene; particularly where the gene is associated with biofuel production.

WO2008108989 (Danisco) entitled “Cultures with improved phage resistance” discloses selecting bacteriophage resistant strains of bacteria and also selecting the strains which have an additional spacer having 100% identity with a region of phage RNA. Improved strain combinations and starter culture rotations are described for use in the dairy industry. Certain phages are described for use as biocontrol agents.

WO2009115861 (Institut Pasteur) entitled “Molecular typing and subtyping of Salmonella by identification of the variable nucleotide sequences of the CRISPR loci” discloses methods for detecting and identifying bacterial of the Salmonella genus by using their variable nucleotide sequences contained in CRISPR loci.

WO2006073445 (Danisco) entitled “Detection and typing of bacterial strains” describes detecting and typing of bacterial strains in food products, dietary supplements and environmental samples. Strains of Lactobacillus are identified through specific CRISPR nucleotide sequences.

Urnov F et al. (2010) entitled “Genome editing with engineered zinc finger nucleases” Nature 11: 636-646 is a review article about zinc finger nucleases and how they have been instrumental in the field of reverse genetics in a range of model organisms. Zinc finger nucleases have been developed so that precisely targeting genome cleavage is possible followed by gene modification in the subsequent repair process. However, zinc finger nucleases are generated by fusing a number of zinc finger DNA-binding domains to a DNA cleavage domain. DNA sequence specificity is achieved by coupling several zinc fingers in series, each recognising a three nucleotide motif. A significant drawback with the technology is that new zinc fingers need to be developed for each new DNA locus which requires to be cleaved. This requires protein engineering and extensive screening to ensure specificity of DNA binding.

In the fields of genetic engineering and genomic research there is an ongoing need for improved agents for sequence/site specific nucleic acid detection and/or cleavage.

The inventors have made a surprising discovery in that certain bacteria expressing Cas3, which has helicase-nuclease activity, express Cas3 as a fusion with Cse1. The inventors have also unexpectedly been able to produce artificial fusions of Cse1 with other nuclease enzymes.

The inventors have also discovered that Cas3-independent target DNA recognition by Cascade marks DNA for cleavage by Cas3, and that Cascade DNA binding is governed by topological requirements of the target DNA.

The inventors have further found that Cascade is unable to bind relaxed target plasmids, but surprisingly Cascade displays high affinity for targets which have a negatively supercoiled (nSC) topology.

Accordingly in a first aspect the present invention provides a clustered regularly interspaced short palindromic repeat (CRISPR)-associated complex for antiviral defence (Cascade), the Cascade protein complex, or portion thereof, comprising at least CRISPR-associated protein subunits:
Cas7 (or COG 1857) having an amino acid sequence of SEQ ID NO:3 or a sequence of at least 18% identity therewith,
Cas5 (or COG1688) having an amino acid sequence of SEQ ID NO:4 or a sequence of at least 17% identity therewith, and
Cas6 (or COG 1583) having an amino acid sequence of SEQ ID NO:5 or a sequence of at least 16% identity therewith;
   and wherein at least one of the subunits includes an additional amino acid sequence providing nucleic acid or chromatin modifying, visualising, transcription activating or transcription repressing activity.

A subunit which includes an additional amino acid sequence having nucleic acid or chromatin modifying, visualising, transcription activating or transcription repressing activity is an example of what may be termed “a subunit linked to at least one functional moiety”; a functional moiety being the polypeptide or protein made up of the additional amino acid sequence. The transcription activating activity may be that leading to activation or upregulation of a desired genes; the transcription repressing activity leading to repressing or downregulation of a desired genes. The selection of the gene being due to the targeting of the cascade complex of the invention with an RNA molecule, as described further below.

The additional amino acid sequence having nucleic acid or chromatin modifying, visualising, transcription activating or transcription repressing activity is preferably formed of contiguous amino acid residues. These additional amino acids may be viewed as a polypeptide or protein which is contiguous and forms part of the Cas or Cse subunit(s) concerned. Such a polypeptide or protein sequence is preferably not normally part of any Cas or Cse subunit amino acid sequence. In other words, the additional amino acid sequence having nucleic acid or chromatin modifying, visualising, transcription activating or transcription repressing activity may be other than a Cas or Cse subunit amino acid sequence, or portion thereof, i.e. may be other than a Cas3 submit amino acid sequence or portion thereof.

The additional amino acid sequence with nucleic acid or chromatin modifying, visualising, transcription activating or transcription repressing activity may, as desired, be obtained or derived from the same organism, e.g. E. coli, as the Cas or Cse subunit(s).

Additionally and/or alternatively to the above, the additional amino acid sequence having nucleic acid or chromatin modifying, visualising, transcription activating or transcription repressing activity may be “heterologous” to the amino acid sequence of the Cas or Cse subunit(s). Therefore, the additional amino acid sequence may be obtained or derived from an organism different from the organism from which the Cas and/or Cse subunit(s) are derived or originate.

Throughout, sequence identity may be determined by way of BLAST and subsequent Cobalt multiple sequence alignment at the National Center for Biotechnology Information webserver, where the sequence in question is compared to a reference sequence (e.g. SEQ ID NO: 3, 4 or 5). The amino acid sequences may be defined in terms of percentage sequence similarity based on a BLOSUM62 matrix or percentage identity with a given reference sequence (e.g. SEQ ID NO:3, 4 or 5). The similarity or identity of a sequence involves an initial step of making the best alignment before calculating the percentage conservation with the reference and reflects a measure of evolutionary relationship of sequences.

Cas7 may have a sequence similarity of at least 31% with SEQ ID NO:3; Cas5 may have a sequence similarity of at least 26% with SEQ ID NO:4. Cas6 may have a sequence similarity of at least 27% with SEQ ID NO:5.

For Cse1/CasA (502 AA):
>gi|16130667|ref|NP_417240.1| CRISP RNA
(crRNA) containing Cascade antiviral
complex protein [Escherichia coli str.
K-12 substr. MG1655]
[SEQ ID NO: 1]
MNLLIDNWIPVRPRNGGKVQIINLQSLYCSRDQWRLSLPRDDMELAALAL
LVCIGQIIAPAKDDVEFRHRIMNPLTEDEFQQLIAPWIDMFYLNHAEHPF
MQTKGVKANDVTPMEKLLAGVSGATNCAFVNQPGQGEALCGGCTAIALFN
QANQAPGFGGGFKSGLRGGTPVTTFVRGIDLRSTVLLNVLTLPRLQKQFP
NESHTENQPTWIKPIKSNESIPASSIGFVRGLFWQPAHIELCDPIGIGKC
SCCGQESNLRYTGFLKEKFTFTVNGLWPHPHSPCLVTVKKGEVEEKFLAF
TTSAPSWTQISRVVVDKIIQNENGNRVAAVVNQFRNIAPQSPLELIMGGY
RNNQASILERRHDVLMFNQGWQQYGNVINEIVTVGLGYKTALRKALYTFA
EGFKNKDFKGAGVSVHETAERHFYRQSELLIPDVLANVNFSQADEVIADL
RDKLHQLCEMLFNQSVAPYAHHPKLISTLALARATLYKHLRELKPQGGPS
NG
For Cse2/CasB (160 AA):
>gi|16130666|ref|NP_417239.1| CRISP RNA
(crRNA) containing Cascade antiviral
complex protein [Escherichia coli str.
K-12 substr. MG1655]
[SEQ ID NO: 2]
MADEIDAMALYRAWQQLDNGSCAQIRRVSEPDELRDIPAFYRLVQPFGWE
NPRHQQALLRMVFCLSAGKNVIRHQDKKSEQTTGISLGRALANSGRINER
RIFQLIRADRTADMVQLRRLLTHAEPVLDWPLMARMLTWWGKRERQQLLE
DFVLTTNKNA
For Cas7/CasC/Cse4 (363 AA):
>gi|16130665|ref|NP_417238.1| CRISP RNA
(crRNA) containing Cascade antiviral
complex protein [Escherichia coli str.
K-12 substr. MG1655]
[SEQ ID NO: 3]
MSNFINIHVLISHSPSCLNRDDMNMQKDAIFGGKRRVRISSQSLKRAMRK
SGYYAQNIGESSLRTIHLAQLRDVLRQKLGERFDQKIIDKTLALLSGKSV
DEAEKISADAVTPWVVGEIAWFCEQVAKAEADNLDDKKLLKVLKEDIAAI
RVNLQQGVDIALSGRMATSGMMTELGKVDGAMSIAHAITTHQVDSDIDWF
TAVDDLQEQGSAHLGTQEFSSGVFYRYANINLAQLQENLGGASREQALEI
ATHVVHMLATEVPGAKQRTYAAFNPADMVMVNFSDMPLSMANAFEKAVKA
KDGFLQPSIQAFNQYWDRVANGYGLNGAAAQFSLSDVDPITAQVKQMPTL
EQLKSWVRNNGEA
For Cas5/CasD (224 AA):
>gi|90111483|ref|NP_417237.2| CRISP RNA
(crRNA) containing Cascade antiviral
complex protein [Escherichia coli str.
K-12 substr. MG1655]
[SEQ ID NO: 4]
MRSYLILRLAGPMQAWGQPTFEGTRPTGRFPTRSGLLGLLGACLGIQRDD
TSSLQALSESVQFAVRCDELILDDRRVSVTGLRDYHTVLGAREDYRGLKS
HETIQTWREYLCDASFTVALWLTPHATMVISELEKAVLKPRYTPYLGRRS
CPLTHPLFLGTCQASDPQKALLNYEPVGGDIYSEESVTGHHLKFTARDEP
MITLPRQFASREWYVIKGGMDVSQ
For Cas6e/CasE (199 AA):
>gi|16130663|ref|NP_417236.1| CRISPR RNA
precursor cleavage enzyme; CRISP RNA
(crRNA) containing Cascade antiviral
complex protein [Escherichia coli str.
K-12 substr. MG1655]
[SEQ ID NO: 5]
MYLSKVIIARAWSRDLYQLHQGLWHLFPNRPDAARDFLFHVEKRNTPEGC
HVLLQSAQMPVSTAVATVIKTKQVEFQLQVGVPLYFRLRANPIKTILDNQ
KRLDSKGNIKRCRVPLIKEAEQIAWLQRKLGNAARVEDVHPISERPQYFS
GDGKSGKIQTVCFEGVLTINDAPALIDLVQQGIGPAKSMGCGLLSLAPL

In defining the range of sequence variants which fall within the scope of the invention, for the avoidance of doubt, the following are each optional limits on the extent of variation, to be applied for each of SEQ ID NO:1, 2, 3, 4 or 5 starting from the respect broadest range of variants as specified in terms of the respective percentage identity above. The range of variants therefore may therefore include: at least 16%, or at least 17%, or at least 18%, or at least 19%, or at least 20%, or at least 21%, or at least 22%, or at least 23%, or at least 24%, or at least 25%, or at least 26%, or at least 27%, or at least 28%, or at least 29%, or at least 30%, or at least 31%, or at least 32%, or at least 33%, or at least 34%, or at least 35%, or at least 36%, or at least 37%, or at least 38%, or at least 39%, or at least 40%, or at least 41%, or at least 42%, or at least 43%, at least 44%, or at least 45%, or at least 46%, or at least 47%, or at least 48%, or at least 49%, or at least 50%, or at least 51%, or at least 52%, or at least 53%, or at least 54%, or at least 55%, or at least 56%, or at least 57%, or at least 58%, or at least 59%, or at least 60%, or at least 61%, or at least 62%, or at least 63%, or at least 64%, or at least 65%, or at least 66%, or at least 67%, or at least 68%, or at least 69%, or at least 70%, or at least 71%, at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or at least 96%, or at least 97%, or at least 98%, or at least 99%, or 100% amino acid sequence identity.

Throughout, the Makarova et al. (2011) nomenclature is being used in the definition of the Cas protein subunits. Table 2 on page 5 of the Makarova et al. article lists the Cas genes and the names of the families and superfamilies to which they belong. Throughout, reference to a Cas protein or Cse protein subunit includes cross reference to the family or superfamily of which these subunits form part.

Throughout, the reference sequences of the Cas and Cse subunits of the invention may be defined as a nucleotide sequence encoding the amino acid sequence. For example, the amino acid sequence of SEQ ID NO:3 for Cas7 also includes all nucleic acid sequences which encode that amino acid sequence. The variants of Cas7 included within the scope of the invention therefore include nucleotide sequences of at least the defined amino acid percentage identities or similarities with the reference nucleic acid sequence; as well as all possible percentage identities or similarities between that lower limit and 100%.

The Cascade complexes of the invention may be made up of subunits derived or modified from more than one different bacterial or archaeal prokaryote. Also, the subunits from different Cas subtypes may be mixed.

In a preferred aspect, the Cas6 subunit is a Cas6e subunit of SEQ ID NO: 17 below, or a sequence of at least 16% identity therewith.

The sequence of a preferred Cas6e subunit is >gi|16130663|ref|NP_417236.1| CRISPR RNA precursor cleavage enzyme; CRISP RNA (crRNA) containing Cascade antiviral complex protein [Escherichia coli str. K-12 substr. MG1655]:

[SEQ ID NO: 17]
MYLSKVIIARAWSRDLYQLHQGLWHLFPNRPDAARDFLFHVEKRNTPEGC
HVLLQSAQMPVSTAVATVIKTKQVEFQLQVGVPLYFRLRANPIKTILDNQ
KRLDSKGNIKRCRVPLIKEAEQIAWLQRKLGNAARVEDVHPISERPQYFS
GDGKSGKIQTVCFEGVLTINDAPALIDLVQQGIGPAKSMGCGLLSLAPL

The Cascade complexes, or portions thereof, of the invention—which comprise at least one subunit which includes an additional amino acid sequence having nucleic acid or chromatin modifying, visualising, transcription activating or transcription repressing activity—may further comprise a Cse2 (or Ygck-like) subunit having an amino acid sequence of SEQ ID NO:2 or a sequence of at least 20% identity therewith, or a portion thereof. Alternatively, the Cse subunit is defined as having at least 38% similarity with SEQ ID NO:2. Optionally, within the protein complex of the invention it is the Cse2 subunit which includes the additional amino acid sequence having nucleic acid or chromatin modifying activity.

Additionally or alternatively, the Cascade complexes of the invention may further comprise a Cse1 (or YgcL-like) subunit having an amino acid sequence of SEQ ID NO: 1 or a sequence of at least 9% identity therewith, or a portion thereof. Optionally within the protein complex of the invention it is the Cse1 subunit which includes the additional amino acid sequence having nucleic acid or chromatin modifying, visualising, transcription activating or transcription repressing activity.

In preferred embodiments, a Cascade complex of the invention is a Type I CRISPR-Cas system protein complex; more preferably a subtype I-E CRISPR-Cas protein complex or it can be based on a Type I-A or Type I-B complex. A Type I-C, D or F complex is possible. In particularly preferred embodiments based on the E. coli system, the subunits may have the following stoichiometries: Cse1₁Cse2₂Cas7₆Cas5₁Cas6₁ or Cse1₁Cse2₂Cas7₆Cas5₁Cas6e₁.

The additional amino acid sequence having nucleic acid or chromatin modifying, visualising, transcription activating or transcription repressing activity may be translationally fused through expression in natural or artificial protein expression systems, or covalently linked by a chemical synthesis step to the at least one subunit; preferably the at least one functional moiety is fused or linked to at least the region of the N terminus and/or the region of the C terminus of at least one of a Cse1, Cse2 Cas7. Cas5. Cas6 or Cas6e subunit. In particularly preferred embodiments, the additional amino acid sequence having nucleic acid or chromatin modifying activity is fused or linked to the N terminus or the C terminus of a Cse1, a Cse2 or a Cas5 subunit; more preferably the linkage is in the region of the N terminus of a Cse1 subunit, the N terminus of a Cse2 subunit, or the N terminus of a Cas7 subunit.

The additional amino acid sequence having nucleic acid or chromatin modifying, activating, repressing or visualising activity may be a protein; optionally selected from a helicase, a nuclease, a nuclease-helicase, a DNA methyltransferase (e.g. Dam), or DNA demethylase, a histone methyltransferase, a histone demethylase, an acetylase, a deacetylase, a phosphatase, a kinase, a transcription (co-)activator, an RNA polymerase submit, a transcription repressor, a DNA binding protein, a DNA structuring protein, a marker protein, a reporter protein, a fluorescent protein, a ligand binding protein (e.g. mCherry or a heavy metal binding protein), a signal peptide (e.g. Tat-signal sequence), a subcellular localisation sequence (e.g. nuclear localisation sequence) or an antibody epitope.

The protein concerned may be a heterologous protein from a species other than the bacterial species from which the Cascade protein subunits have their sequence origin.

When the protein is a nuclease, it may be one selected from a type II restriction endonuclease such as FokI, or a mutant or an active portion thereof. Other type II restriction endonucleases which may be used include EcoR1, EcoRV, BgII, BamHI, BsgI and BspMI. Preferably, one protein complex of the invention may be fused to the N terminal domain of FokI and another protein complex of the invention may be fused to the C terminal domain of FokI. These two protein complexes may then be used together to achieve an advantageous locus specific double stranded cut in a nucleic acid, whereby the location of the cut in the genetic material is at the design and choice of the user, as guided by the RNA component (defined and described below) and due to presence of a so-called “protospacer adjacent motif”' (PAM) sequence in the target nucleic acid strand (also described in more detail below).

In a preferred embodiment, a protein complex of the invention has an additional amino acid sequence which is a modified restriction endonuclease, e.g. FokI. The modification is preferably in the catalytic domain. In preferred embodiments, the modified FokI is KKR Sharkey or ELD Sharkey which is fused to the Cse1 protein of the protein complex. In a preferred application of these complexes of the invention, two of these complexes (KKR Sharkey and ELD Sharkey) may be together in combination. A heterodimer pair of protein complexes employing differently modified FokI is has particular advantage in targeted double stranded cutting of nucleic acid. If homodimers are used then it is possible that there is more cleavage at non-target sites due to non-specific activity. A heterodimer approach advantageously increases the fidelity of the cleavage in a sample of material.

The Cascade complex with additional amino acid sequence having nucleic acid or chromatin modifying, visualising, transcription activating or transcription repressing activity defined and described above is a component part of an overall system of the invention which advantageously permits the user to select in a predetermined matter a precise genetic locus which is desired to be cleaved, tagged or otherwise altered in some way, e.g methylation, using any of the nucleic acid or chromatin modifying, visualising, transcription activating or transcription repressing entities defined herein. The other component part of the system is an RNA molecule which acts as a guide for directing the Cascade complex of the invention to the correct locus on DNA or RNA intending to be modified, cut or tagged.

The Cascade complex of the invention preferably also comprises an RNA molecule which comprises a ribonucleotide sequence of at least 50% identity to a desired target nucleic acid sequence, and wherein the protein complex and the RNA molecule form a ribonucleoprotein complex. Preferably the ribonucleoprotein complex forms when the RNA molecule is hybridized to its intended target nucleic acid sequence. The ribonucleoprotein complex forms when the necessary components of Cascade-functional moiety combination and RNA molecule and nucleic acid (DNA or RNA) are present together in suitable physiological conditions, whether in vivo or in vitro. Without wishing to be bound by any particular theory, the inventors believe that in the context of dsDNA, particularly negatively supercoiled DNA, the Cascade complex associating with the dsDNA causes a partial unwinding of the duplex strands which then allows the RNA to associate with one strand; the whole ribonucleoprotein complex then migrates along the DNA strand until a target sequence substantially complementary to at least a portion of the RNA sequence is reached, at which point a stable interaction between RNA and DNA strand occurs, and the function of the functional moiety takes effect, whether by modifying, nuclease cutting or tagging of the DNA at that locus.

In preferred embodiments, a portion of the RNA molecule has at least 50% identity to the target nucleic acid sequence; more preferably at least 95% identity to the target sequence. In more preferred embodiments, the portion of the RNA molecule is substantially complementary along its length to the target DNA sequence; i.e. there is only one, two, three, four or five mismatches which may be contiguous or non-contiguous. The RNA molecule (or portion thereof) may have at least 51%, or at least 52%, or at least 53%, or at least 54%, or at least 55%, or at least 56%, or at least 57%, or at least 58%, or at least 59%, or at least 60%, or at least 61%, or at least 62%, or at least 63%, or at least 64%, or least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or least 85%, or at least 86%, or at least 87%, or at least 88%, or at least 89%, or at least 90%, or at least 91%, or at least 92%, or at least 93%, or at least 94%, or at least 95%, or at least 96%, or at least 97%, or at least 98%, or at least 99%, or 100% identity to the target sequence.

The target nucleic acid may be DNA (ss or ds) or RNA.

In other preferred embodiments, the RNA molecule or portion thereof has at least 70% identity with the target nucleic acid. At such levels of identity, the target nucleic acid is preferably dsDNA.

The RNA molecule will preferably require a high specificity and affinity for the target nucleic acid sequence. A dissociation constant (K_{d}) in the range 1 pM to 1 μM, preferably 1-100 nM is desirable as determined by preferably native gel electrophoresis, or alternatively isothermal titration calorimetry, surface plasmon resonance, or fluorescence based titration methods. Affinity may be determined using an electrophoretic mobility shift assay (EMSA), also called gel retardation assay (see Semenova E et al. (2011) Proc. Natl. Acad. Sci. USA 108: 10098 -10103).

The RNA molecule is preferably modelled on what are known from nature in prokaryotes as CRISPR RNA (crRNA) molecules. The structure of crRNA molecules is already established and explained in more detail in Jore et al. (2011) Nature Structural & Molecular Biology 18: 529-537. In brief, a mature crRNA of type I-E is often 61 nucleotides long and consists of a 5′ “handle” region of 8 nucleotides, the “spacer” sequence of 32 nucleotides, and a 3′ sequence of 21 nucleotides which form a hairpin with a tetranucleotide loop. However, the RNA used in the invention does not have to be designed strictly to the design of naturally occurring crRNA, whether in length, regions or specific RNA sequences. What is clear though, is that RNA molecules for use in the invention may be designed based on gene sequence information in the public databases or newly discovered, and then made artificially, e.g. by chemical synthesis in whole or in part. The RNA molecules of the invention may also be designed and produced by way of expression in genetically modified cells or cell free expression systems and this option may include synthesis of some or all of the RNA sequence.

The structure and requirements of crRNA has also been described in Semenova E et al. (2011) Proc. Natl. Acad. Sci. USA 108: 10098-10103. There is a so-called “SEED” portion forming the 5′ end of the spacer sequence and which is flanked 5′ thereto by the 5′ handle of 8 nucleotides. Semenova et al. (2011) have found that all residues of the SEED sequence should be complementary to the target sequence, although for the residue at position 6, a mismatch may be tolerated. Similarly, when designing and making an RNA component of a ribonucleoprotein complex of the invention directed at a target locus (i.e. sequence), the necessary match and mismatch rules for the SEED sequence can be applied.

The invention therefore includes a method of detecting and/or locating a single base change in a target nucleic acid molecule comprising contacting a nucleic acid sample with a ribonucleoprotein complex of the invention as hereinbefore described, or with a Cascade complex and separate RNA component of the invention as hereinbefore described, and wherein the sequence of the RNA component (including when in the ribonucleoprotein complex) is such that it discriminates between a normal allele and a mutant allele by virtue of a single base change at position 6 of a contiguous sequence of 8 nucleotide residues.

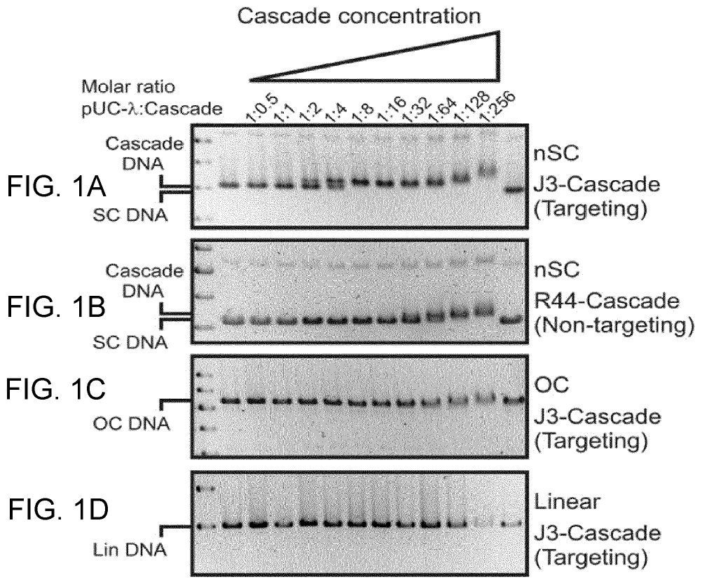

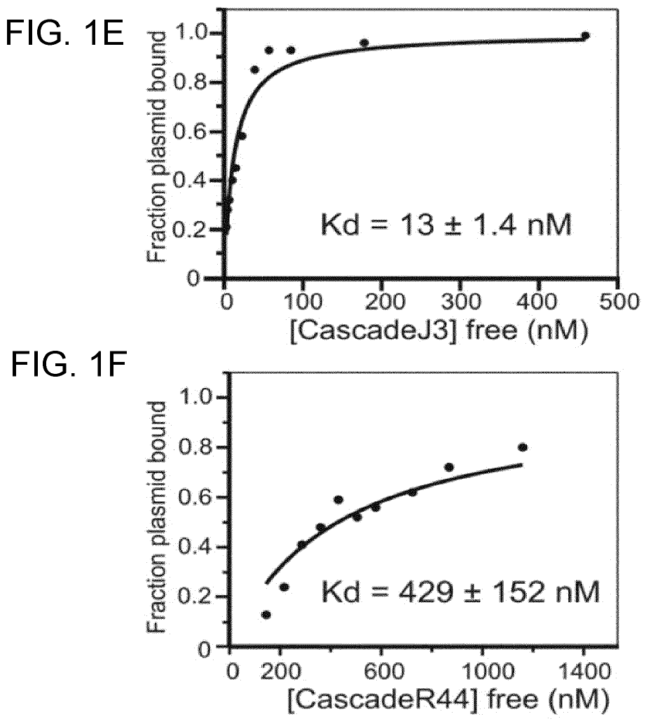

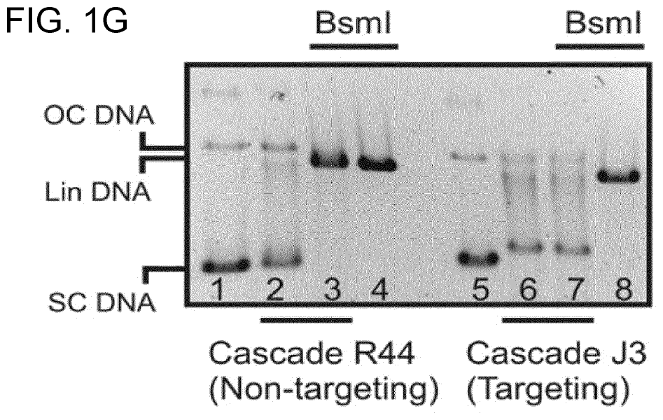

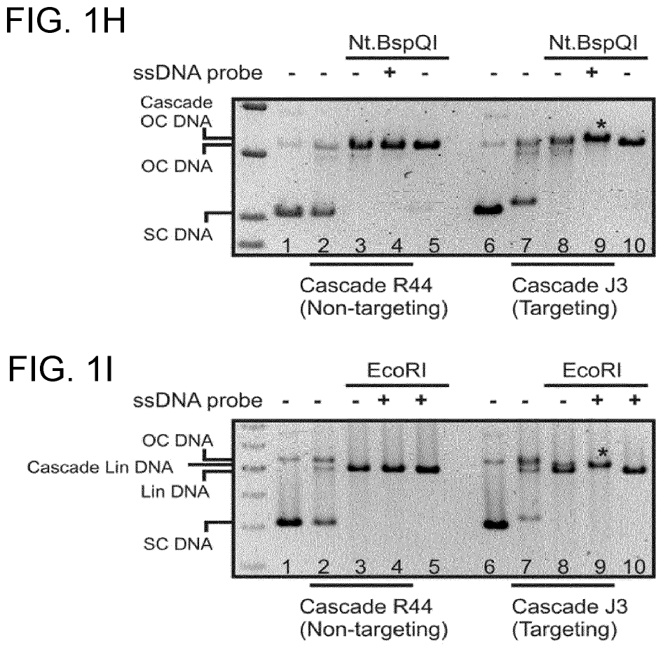

In embodiments of the invention, the RNA molecule may have a length in the range of 35-75 residues. In preferred embodiments, the portion of the RNA which is complementary to and used for targeting a desired nucleic acid sequence is 32 or 33 residues long. (In the context of a naturally occurring crRNA, this would correspond to the spacer portion; as shown in FIG. 1 of Semenova et al. (2011)).

A ribonucleoprotein complex of the invention may additionally have an RNA component comprising 8 residues 5′ to the RNA sequence which has at least substantial complementarity to the nucleic acid target sequence. (The RNA sequence having at least substantial complementarity to the nucleic acid target sequence would be understood to correspond in the context of a crRNA as being the spacer sequence. The 5′ flanking sequence of the RNA would be considered to correspond to the 5′ handle of a crRNA. This is shown in FIG. 1 of Semenova et al. (2011)).

A ribonucleoprotein complex of the invention may have a hairpin and tetranucleotide loop forming sequence 3′ to the RNA sequence which has at least substantial complementarity to the DNA target sequence. (In the context of crRNA, this would correspond to a 3′ handle flanking the spacer sequence as shown in FIG. 1 of Semenova et al. (2011)).

In some embodiments, the RNA may be a CRISPR RNA (crRNA).

The Cascade proteins and complexes of the invention may be characterised in vitro in terms of its activity of association with the RNA guiding component to form a ribonucleoprotein complex in the presence of the target nucleic acid (which may be DNA or RNA). An electrophoretic mobility shift assay (EMSA) may be used as a functional assay for interaction of complexes of the invention with their nucleic acid targets. Basically, Cascade-functional moiety complex of the invention is mixed with nucleic acid targets and the stable interaction of the Cascade-functional moiety complex is monitored by EMSA or by specific readout out the functional moiety, for example endonucleolytic cleavage of target DNA at the desired site. This can be determined by further restriction fragment length analysis using commercially available enzymes with known specificities and cleavage sites in a target DNA molecule.

Visualisation of binding of Cascade proteins or complexes of the invention to DNA or RNA in the presence of guiding RNA may be achieved using scanning/atomic force microscopy (SFM/AFM) imaging and this may provide an assay for the presence of functional complexes of the invention.

The invention also provides a nucleic acid molecule encoding at least one clustered regularly interspaced short palindromic repeat (CRISPR)-associated protein subunit selected from:
a. a Cse1 subunit having an amino acid sequence of SEQ ID NO: 1 or a sequence of at least 9% identity therewith;
b. a Cse2 subunit having an amino acid sequence of SEQ ID NO:2 or a sequence of at least 20% identity therewith;
c. a Cas7 subunit having an amino acid sequence of SEQ ID NO:3 or a sequence of at least 18% identity therewith;
d. a Cas5 subunit having an amino acid sequence of SEQ ID NO:4 or a sequence of at least 17% identity therewith;
e. a Cas6 subunit having an amino acid sequence of SEQ ID NO:5 or a sequence of at least 16% identity therewith; and
   wherein at least a, b, c, d or e includes an additional amino acid sequence having nucleic acid or chromatin modifying, visualising, transcription activating or transcription repressing activity.

The additional amino acid sequence having nucleic acid or chromatin modifying, visualising, transcription activating or transcription repressing activity is preferably fused to the CRISPR-associated protein subunit.

In the nucleic acids of the invention defined above, the nucleotide sequence may be that which encodes the respective SEQ ID NO: 1, SEQ ID NO:2, SEQ ID NO:3, SEQ ID NO:4 or SEQ ID NO:5, or in defining the range of variant sequences thereto, it may be a sequence hybridisable to that nucleotide sequence, preferably under stringent conditions, more preferably very high stringency conditions. A variety of stringent hybridisation conditions will be familiar to the skilled reader in the field. Hybridization of a nucleic acid molecule occurs when two complementary nucleic acid molecules undergo an amount of hydrogen bonding to each other known as Watson-Crick base pairing. The stringency of hybridization can vary according to the environmental (i.e. chemical/physical/biological) conditions surrounding the nucleic acids, temperature, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, NY, 2001); and Tijssen, Laboratory Techniques in Biochemistry and Molecular Biology—Hybridization with Nucleic Acid Probes Part I, Chapter 2 (Elsevier, New York, 1993). The Tₘ is the temperature at which 50% of a given strand of a nucleic acid molecule is hybridized to its complementary strand. The following is an exemplary set of hybridization conditions and is not limiting:

### Very High Stringency (Allows Sequences That Share at Least 90% Identity to Hybridize)

| | |
|---|---|
| Hybridization: | 5x SSC at 65° C. for 16 hours |
| Wash twice: | 2x SSC at room temperature (RT) for 15 minutes each |
| Wash twice: | 0.5x SSC at 65° C. for 20 minutes each |

### High Stringency (Allows Sequences That Share at Least 80% Identity to Hybridize)

| | |
|---|---|
| Hybridization: | 5x-6x SSC at 65°C-70° C. for 16-20 hours |
| Wash twice: | 2x SSC at RT for 5-20 minutes each |
| Wash twice: | 1x SSC at 55°C-70° C. for 30 minutes each |

### Low Stringency (Allows Sequences That Share at Least 50% Identity to Hybridize)

| | |
|---|---|
| Hybridization: | 6x SSC at RT to 55° C. for 16-20 hours |
| Wash at least twice: | 2x-3x SSC at RT to 55ºC for 20-30 minutes each. |

The nucleic acid molecule may be an isolated nucleic acid molecule and may be an RNA or a DNA molecule.

The additional amino acid sequence may be selected from a helicase, a nuclease, a nuclease-helicase (e.g. Cas3), a DNA methyltransferase (e.g. Dam), a DNA demethylase, a histone methyltransferase, a histone demethylase, an acetylase, a deacetylase, a phosphatase, a kinase, a transcription (co-)activator, an RNA polymerase subunit, a transcription repressor, a DNA binding protein, a DNA structuring protein, a marker protein, a reporter protein, a fluorescent protein, a ligand binding protein (e.g. mCherry or a heavy metal binding protein), a signal peptide (e.g. Tat-signal sequence), a subcellular localisation sequence (e.g. nuclear localisation sequence), or an antibody epitope. The additional amino acid sequence may be, or from a different protein from the organism from which the relevant Cascade protein subunit(s) are derived.

The invention includes an expression vector comprising a nucleic acid molecule as hereinbefore defined. One expression vector may contain the nucleotide sequence encoding a single Cascade protein subunit and also the nucleotide sequence encoding the additional amino acid sequence, whereby on expression the subunit and additional sequence are fused. Other expression vectors may comprise nucleotide sequences encoding just one or more Cascade protein subunits which are not fused to any additional amino acid sequence.

The additional amino acid sequence with nucleic acid or chromatin modifying activity may be fused to any of the Cascade subunits via a linker polypeptide. The linker may be of any length up to about 60 or up to about 100 amino acid residues. Preferably the linker has a number of amino acids in the range 10 to 60, more preferably 10-20. The amino acids are preferably polar and/or small and/or charged amino acids (e.g. Gln, Ser, Thr, Pro, Ala, Glu, Asp, Lys, Arg, His, Asn, Cys, Tyr). The linker peptide is preferably designed to obtain the correct spacing and positioning of the fused functional moiety and the subunit of Cascade to which the moiety is fused to allow proper interaction with the target nucleotide.

An expression vector of the invention (with or without nucleotide sequence encoding amino acid residues which on expression will be fused to a Cascade protein subunit) may further comprise a sequence encoding an RNA molecule as hereinbefore defined. Consequently, such expression vectors can be used in an appropriate host to generate a ribonucleoprotein of the invention which can target a desired nucleotide sequence.

Accordingly, the invention also provides a method of modifying, visualising, or activating or repressing transcription of a target nucleic acid comprising contacting the nucleic acid with a ribonucleoprotein complex as hereinbefore defined. The modifying may be by cleaving the nucleic acid or binding to it.

The invention also includes a method of modifying, visualising, or activating or repressing transcription of a target nucleic acid comprising contacting the nucleic acid with a Cascade protein complex as hereinbefore defined, plus an RNA molecule as hereinbefore defined.

In accordance with the above methods, the modification, visualising, or activating or repressing transcription of a target nucleic acid may therefore be carried out in vitro and in a cell free environment; i.e. the method is carried out as a biochemical reaction whether free in solution or whether involving a solid phase. Target nucleic acid may be bound to a solid phase, for example.

In a cell free environment, the order of adding each of the target nucleic acid, the Cascade protein complex and the RNA molecule is at the option of the average skilled person. The three components may be added simultaneously, sequentially in any desired order, or separately at different times and in a desired order. Thus it is possible for the target nucleic acid and RNA to be added simultaneously to a reaction mix and then the Cascade protein complex of the invention to be added separately and later in a sequence of specific method steps.

The modification, visualising, or activating or repressing transcription of a target nucleic acid may be made in situ in a cell, whether an isolated cell or as part of a multicellular tissue, organ or organism. Therefore in the context of whole tissue and organs, and in the context of an organism, the method can be carried out in vivo or it can be carried out by isolating a cell from the whole tissue, organ or organism and then returning the cell treated with ribonucleoprotein complex to its former location, or a different location, whether within the same or a different organism. Thus the method would include allografts, autografts, isografts and xenografts.

In these embodiments, the ribonucleoprotein complex or the Cascade protein complex of the invention requires an appropriate form of delivery into the cell, which will be well known to persons of skill in the art, including microinjection, whether into the cell cytoplasm or into the nucleus.

Also when present separately, the RNA molecule requires an appropriate form of delivery into a cell, whether simultaneously, separately or sequentially with the Cascade protein complex. Such forms of introducing RNA into cells are well known to a person of skill in the art and may include in vitro or ex vivo delivery via conventional transfection methods. Physical methods, such as microinjection and electroporation, as well as calcium co-precipitation, and commercially available cationic polymers and lipids, and cell-penetrating peptides, cell-penetrating particles (gene-gun) may each be used. For example, viruses may be used as delivery vehicles, whether to the cytoplasm and/or nucleus—e.g. via the (reversible) fusion of Cascade protein complex of the invention or a ribonucleoprotein complex of the invention to the viral particle. Viral delivery (e.g. adenovirus delivery) or Agrobacterium-mediated delivery may be used.

The invention also includes a method of modifying visualising, or activating or repressing transcription of a target nucleic acid in a cell, comprising transfecting, transforming or transducing the cell with any of the expression vectors as hereinbefore described. The methods of transfection, transformation or transduction are of the types well known to a person of skill in the art. Where there is one expression vector used to generate expression of a Cascade complex of the invention and when the RNA is added directly to the cell then the same or a different method of transfection, transformation or transduction may be used. Similarly, when there is one expression vector being used to generate expression of a Cascade-functional fusion complex of the invention and when another expression vector is being used to generate the RNA in situ via expression, then the same or a different method of transfection, transformation or transduction may be used.

In other embodiments, mRNA encoding the Cascade complex of the invention is introduced into a cell so that the Cascade complex is expressed in the cell. The RNA which guides the Cascade complex to the desired target sequence is also introduced into the cell, whether simultaneously, separately or sequentially from the mRNA, such that the necessary ribonucleoprotein complex is formed in the cell.

In the aforementioned methods of modifying or visualising a target nucleic acid, the additional amino acid sequence may be a marker and the marker associates with the target nucleic acid; preferably wherein the marker is a protein; optionally a fluorescent protein, e.g. green fluorescent protein (GFP) or yellow fluorescent protein (YFP) or mCherry. Whether in vitro, ex vivo or in vitro, then methods of the invention can be used to directly visualise a target locus in a nucleic acid molecule, preferably in the form of a higher order structure such as a supercoiled plasmid or chromosome, or a single stranded target nucleic acid such as mRNA. Direct visualisation of a target locus may use electron micrography, or fluorescence microscopy.

Other kinds of label may be used to mark the target nucleic acid including organic dye molecules, radiolabels and spin labels which may be small molecules.

In methods of the invention described above, the target nucleic acid is DNA; preferably dsDNA although the target can be RNA; preferably mRNA.

In methods of the invention for modifying, visualising, activating transcription or repressing transcription of a target nucleic acid wherein the target nucleic acid is dsDNA, the additional amino acid sequence with nucleic acid or chromatin modifying activity may be a nuclease or a helicase-nuclease, and the modification is preferably a single stranded or a double stranded break at a desired locus. In this way unique sequence specific cutting of DNA can be engineered by using the Cascade-functional moiety complexes. The chosen sequence of the RNA component of the final ribonucleoprotein complex provides the desired sequence specificity for the action of the additional amino acid sequence.

Therefore, the invention also provides a method of non-homologous end joining of a dsDNA molecule in a cell at a desired locus to remove at least a part of a nucleotide sequence from the dsDNA molecule; optionally to knockout the function of a gene or genes, wherein the method comprises making double stranded breaks using any of the methods of modifying a target nucleic acid as hereinbefore described.

The invention further provides a method of homologous recombination of a nucleic acid into a dsDNA molecule in a cell at a desired locus in order to modify an existing nucleotide sequence or insert a desired nucleotide sequence, wherein the method comprises making a double or single stranded break at the desired locus using any of the methods of modifying a target nucleic acid as hereinbefore described.

The invention therefore also provides a method of modifying, activating or repressing gene expression in an organism comprising modifying, activating transcription or repressing transcription of a target nucleic acid sequence according to any of the methods hereinbefore described, wherein the nucleic acid is dsDNA and the functional moiety is selected from a DNA modifying enzyme (e.g. a demethylase or deacetylase), a transcription activator or a transcription repressor.

The invention additionally provides a method of modifying, activating or repressing gene expression in an organism comprising modifying, activating transcription or repressing transcription of a target nucleic acid sequence according to any of the methods hereinbefore described, wherein the nucleic acid is an mRNA and the functional moiety is a ribonuclease; optionally selected from an endonuclease, a 3′ exonuclease or a 5′ exonuclease.

In any of the methods of the invention as described above, the cell which is subjected to the method may be a prokaryote. Similarly, the cell may be a eukaryotic cell, e.g. a plant cell, an insect cell, a yeast cell, a fungal cell, a mammalian cell or a human cell. When the cell is of a mammal or human then it can be a stem cell (but may not be any human embryonic stem cell). Such stem cells for use in the invention are preferably isolated stem cells. Optionally in accordance with any method the invention a cell is transfected in vitro.

Preferably though, in any of the methods of the invention, the target nucleic acid has a specific tertiary structure, optionally supercoiled, more preferably wherein the target nucleic acid is negatively supercoiled. Advantageously, the ribonucleoprotein complexes of the invention, whether produced in vitro, or whether formed within cells, or whether formed within cells via expression machinery of the cell, can be used to target a locus which would otherwise be difficult to get access to in order to apply the functional activity of a desired component, whether labelling or tagging of a specific sequence, modification of nucleic acid structure, switching on or off of gene expression, or of modification of the target sequence itself involving single or double stranded cutting followed by insertion of one or more nucleotide residues or a cassette.

The invention also includes a pharmaceutical composition comprising a Cascade protein complex or a ribonucleoprotein complex of the invention as hereinbefore described.

The invention further includes a pharmaceutical composition comprising an isolated nucleic acid or an expression vector of the invention as hereinbefore described.

Also provided is a kit comprising a Casacade protein complex of the invention as hereinbefore described plus an RNA molecule of the invention as hereinbefore described.

The invention includes a Cascade protein complex or a ribonucleoprotein complex or a nucleic acid or a vector, as hereinbefore described for use as a medicament.

The invention allows a variety of possibilities to physically alter DNA of prokaryotic or eukaryotic hosts at a specified genomic locus, or change expression patterns of a gene at a given locus. Host genomic DNA can be cleaved or modified by methylation, visualized by fluorescence, transcriptionally activated or repressed by functional domains such as nucleases, methylases, fluorescent proteins, transcription activators or repressors respectively, fused to suitable Cascade-subunits. Moreover, the RNA-guided RNA-binding ability of Cascade permits the monitoring of RNA trafficking in live cells using fluorescent Cascade fusion proteins, and provides ways to sequester or destroy host mRNAs causing interference with gene expression levels of a host cell.

In any of the methods of the invention, the target nucleic acid may be defined, preferably so if dsDNA, by the presence of at least one of the following nucleotide triplets: 5′-CTT-3′, 5′-CAT-3′, 5′-CCT-3′, or 5′-CTC-3′ (or 5′-CUU-3′, 5′-CAU-3′, 5′-CCU-3′, or 5′-CTC-3′ if the target is an RNA). The location of the triplet is in the target strand adjacent to the sequence to which the RNA molecule component of a ribonucleoprotein of the invention hybridizes. The triplet marks the point in the target strand sequence at which base pairing with the RNA molecule component of the ribonucleoprotein does not take place in a 5′ to 3′ (downstream) direction of the target (whilst it takes place upstream of the target sequence from that point subject to the preferred length of the RNA sequence of the RNA molecule component of the ribonucleoprotein of the invention). In the context of a native type I CRISPR system, the triplets correspond to what is known as a “PAM” (protospacer adjacent motif). For ssDNA or ssRNA targets, presence of one of the triplets is not so necessary.

The invention will now be described in detail and with reference to specific examples and drawings in which:

FIGS. 1A-1H show the results of gel-shift assays where Cascade binds negatively supercoiled (nSC) plasmid DNA but not relaxed DNA. FIG. 1A Gel-shift of nSC plasmid DNA with J3-Cascade, containing a targeting (J3) crRNA. pUC-λ was mixed with 2-fold increasing amounts of J3-Cascade, from a pUC-λ: Cascade molar ratio of 1:0.5 up to a 1:256 molar ratio. The first and last lanes contain only pUC-λ. FIG. 1B Gel-shift as in (A) with R44-Cascade containing a non-targeting (R44) cRNA. FIG. 1C Gel-shift as in (A) with Nt.BspQI nicked pUC-λ. FIG. 1D Gel-shift as in (A) with PdmI linearized pUC-λ. FIG. 1E Fit of the fraction pUC-λ bound to J3-Cascade plotted against the concentration of free J3-Cascade gives the dissociation constant (Kd) for specific binding. FIG. 1F Fit of the fraction pUC-λ bound to R44-Cascade plotted against the concentration of free R44-Cascade gives the dissociation constant (Kd) for non-specific binding. FIG. 1G Specific binding of Cascade to the protospacer monitored by restriction analysis, using the unique BsmI restriction site in the protospacer sequence. Lane 1 and 5 contain only pUC-λ. Lane 2 and 6 contain pUC-λ mixed with Cascade. Lane 3 and 7 contain pUC-λ mixed with Cascade and subsequent BsmI addition. Lane 4 and 8 contain pUC-λ mixed with BsmI. FIG. 1H Gel-shift of pUC-λ bound to Cascade with subsequent Nt.BspQI cleavage of one strand of the plasmid. Lane 1 and 6 contain only pUC-λ. Lane 2 and 7 contain pUC-λ mixed with Cascade. Lane 3 and 8 contain pUC-λ mixed with Cascade and subsequent Nt.BspQI nicking. Lane 4 and 9 contain pUC-λ mixed with Cascade, followed by addition of a ssDNA probe complementary to the displaced strand in the R-loop and subsequent nicking with Nt.BspQI. Lane 5 and 10 contain pUC-λ nicked with Nt.BspQI. FIG. 1I Gel-shift of pUC-λ bound to Cascade with subsequent EcoRI cleavage of both strands of the plasmid. Lane 1 and 6 contain only pUC-λ. Lane 2 and 7 contain pUC-λ mixed with Cascade. Lane 3 and 8 contain pUC-λ mixed with Cascade and subsequent EcoRI cleavage. Lane 4 and 9 contain pUC-λ mixed with Cascade, followed by addition of a ssDNA probe complementary to the displaced strand in the R-loop and subsequent cleavage with EcoRI. Lane 5 and 10 contain pUC-λ cleaved with EcoRI.

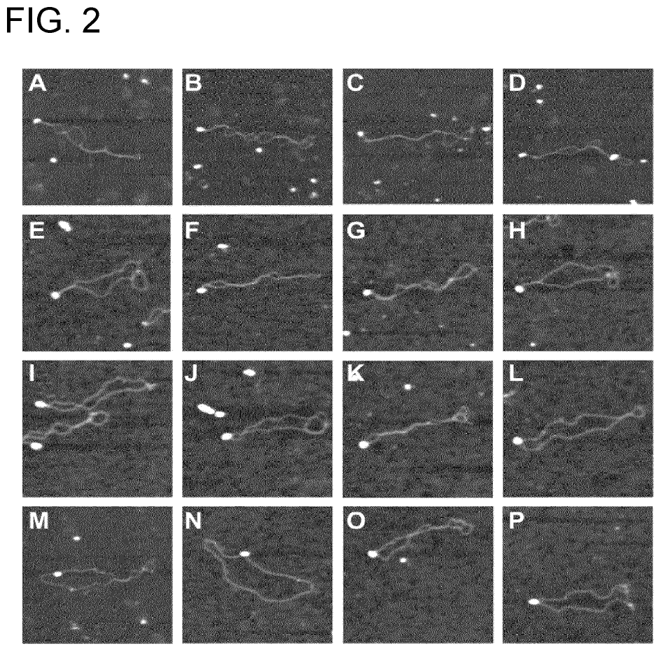

FIG. 2 shows scanning force micrographs demonstrating how Cascade induces bending of target DNA upon protospacer binding. Panels A-P: Scanning force microscopy images of nSC plasmid DNA with J3-Cascade containing a targeting (J3) crRNA. pUC-λ was mixed with J3-Cascade at a pUC-λ: Cascade ratio of 1:7. Each image shows a 500×500 nm surface area. White dots correspond to Cascade.

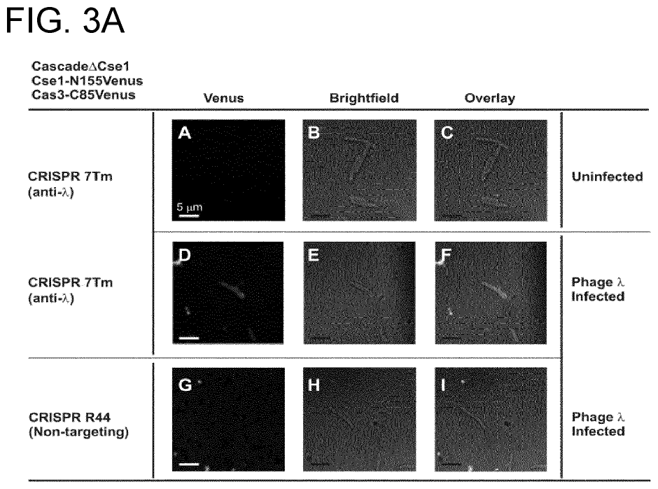

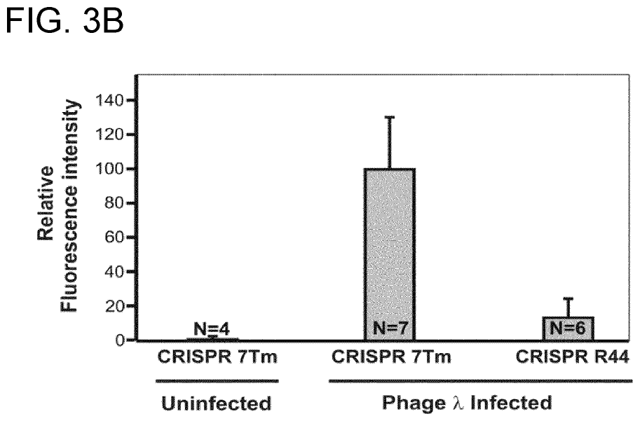

FIG. 3A, panels A-I shows how BiFC analysis reveals that Cascade and Cas3 interact upon target recognition. Panel A: Venus fluorescence of cells expressing CascadeΔCse1 and CRISPR 7Tm, which targets 7 protospacers on the phage λ genome, and Cse1-N155Venus and Cas3-C85Venus fusion proteins. Panel B: Brightfield image of the cells in (A). Panel C: Overlay of (A) and (B). Panel D: Venus fluorescence of phage A infected cells expressing CascadeΔCse1 and CRISPR 7Tm, and Cse1-N155Venus and Cas3-C85Venus fusion proteins. Panel E: Brightfield image of the cells in (D). Panel F: Overlay of (D) and (E). Panel G: Venus fluorescence of phage λ infected cells expressing CascadeΔCse1 and non-targeting CRISPR R44, and N155Venus and C85Venus proteins. Panel H: Brightfield image of the cells in (H). Panel I: Overlay of (G) and (H). FIG. 3B: average of the fluorescence intensity of 4-7 individual cells of each strain, as determined using the profile tool of LSM viewer (Carl Zeiss).

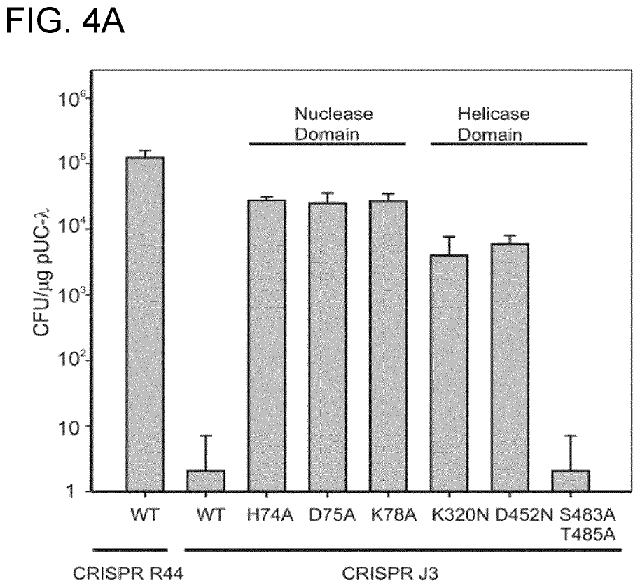

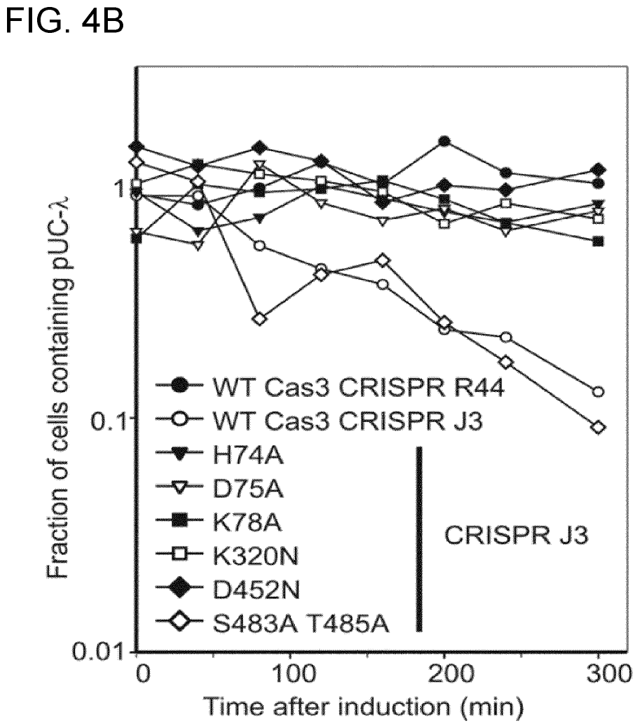

FIGS. 4A and 4B show Cas3 nuclease and helicase activities during CRISPR-interference. FIG. 4A: Competent BL21-AI cells expressing Cascade, a Cas3 mutant and CRISPR J3 were transformed with pUC-λ. Colony forming units per microgram pUC-λ (cfu/ug DNA) are depicted for each of the strains expressing a Cas3 mutant. Cells expressing wt Cas3 and CRISPR J3 or CRISPR R44 serve as positive and negative controls, respectively. FIG. 4B: BL21-AI cells carrying Cascade, Cas3 mutant, and CRISPR encoding plasmids as well as pUC-λ are grown under conditions that suppress expression of the cas genes and CRISPR. At t=0 expression is induced. The percentage of cells that lost pUC-λ over time is shown, as determined by the ratio of ampicillin sensitive and ampicillin resistant cells.

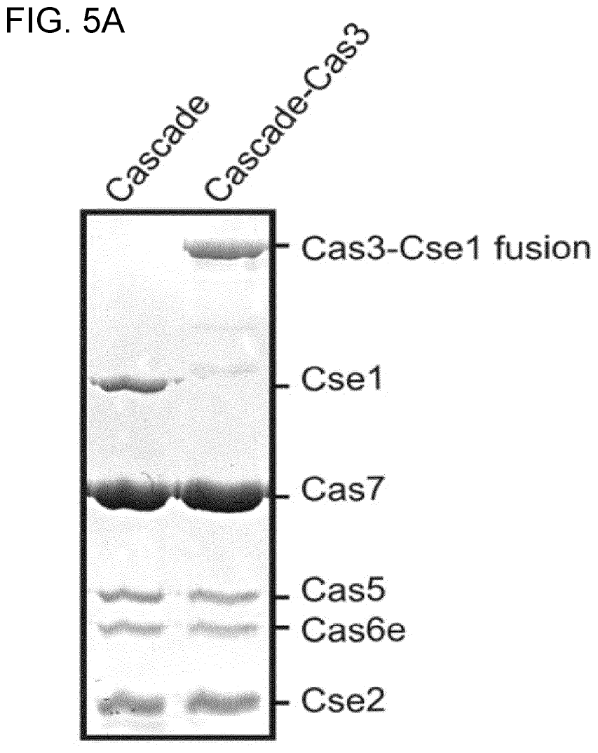

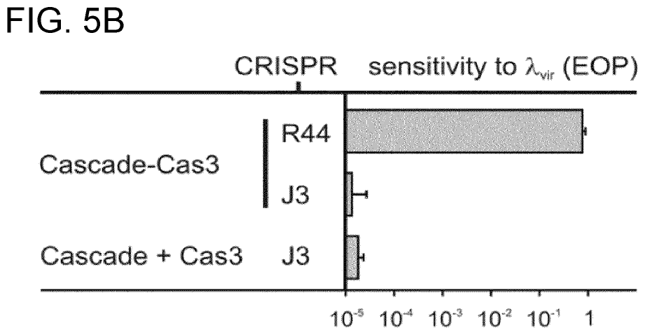

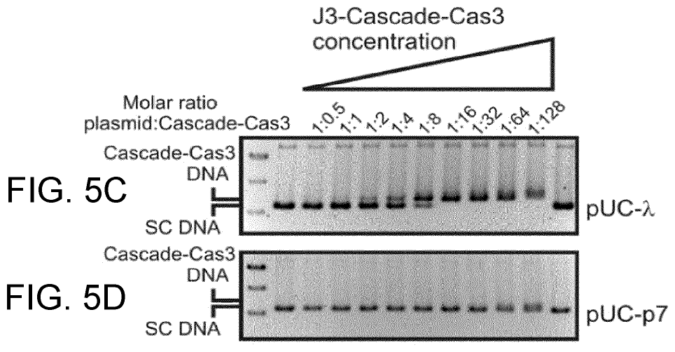

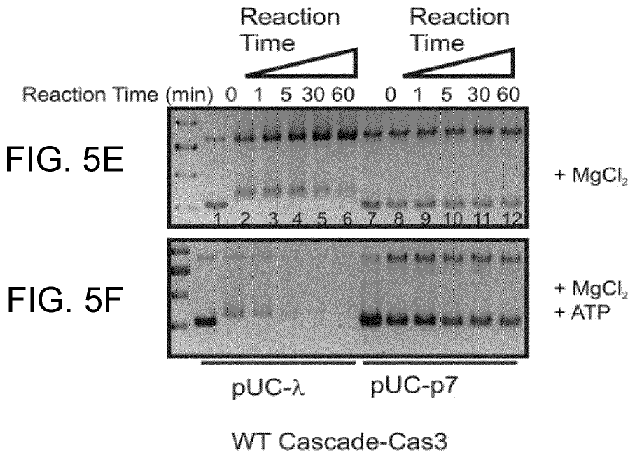

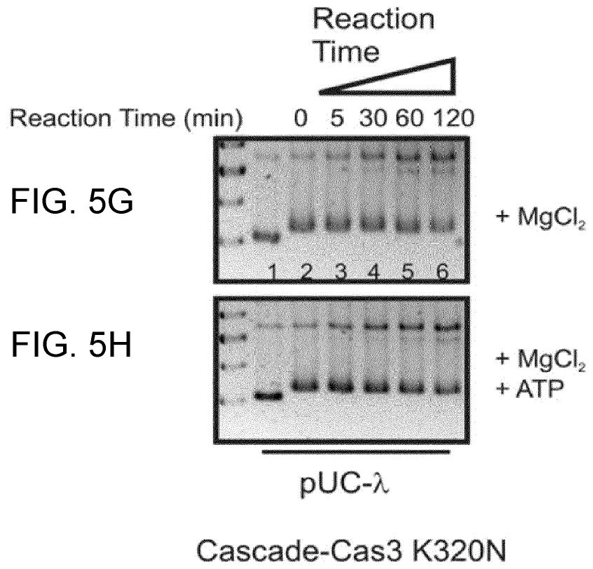

FIGS. 5A-5H show how a Cascade-Cas3 fusion complex provides in vivo resistance and has in vitro nuclease activity. FIG. 5A: Coomassie Blue stained SDS-PAGE of purified Cascade and Cascade-Cas3 fusion complex. FIG. 5B: Efficiency of plaquing of phage λ on cells expressing Cascade-Cas3 fusion complex and a targeting (J3) or non-targeting (R44) CRISPR and on cells expressing Cascade and Cas3 separately together with a targeting (J3) CRISPR. FIG. 5C: Gel-shift (in the absence of divalent metal ions) of nSC target plasmid with J3-Cascade-Cas3 fusion complex. pUC-λ was mixed with 2-fold increasing amounts of J3-Cascade-Cas3, from a pUC-λ:J3-Cascade-Cas3 molar ratio of 1:0.5 up to a 1:128 molar ratio. The first and last lane contain only pUC-λ. FIG. 5D: Gel-shift (in the absence of divalent metal ions) of nSC non-target plasmid with J3-Cascade-Cas3 fusion complex. pUC-p7 was mixed with 2-fold increasing amounts of J3-Cascade-Cas3, from a pUC-p7:J3-Cascade-Cas3 molar ratio of 1:0.5 up to a 1:128 molar ratio. The first and last lane contain only pUC-p7. FIG. 5E: Incubation of nSC target plasmid (pUC-λ, left) or nSC non-target plasmid (pUC-p7, right) with J3-Cascade-Cas3 in the presence of 10 mM MgCl₂. Lane 1 and 7 contain only plasmid. FIG. 5F: Assay as in (E) in the presence of 2 mM ATP. FIG. 5G: Assay as in (E) with the mutant J3-Cascade-Cas3K320N complex. FIG. 5H: Assay as in (G) in the presence of 2 mM ATP.

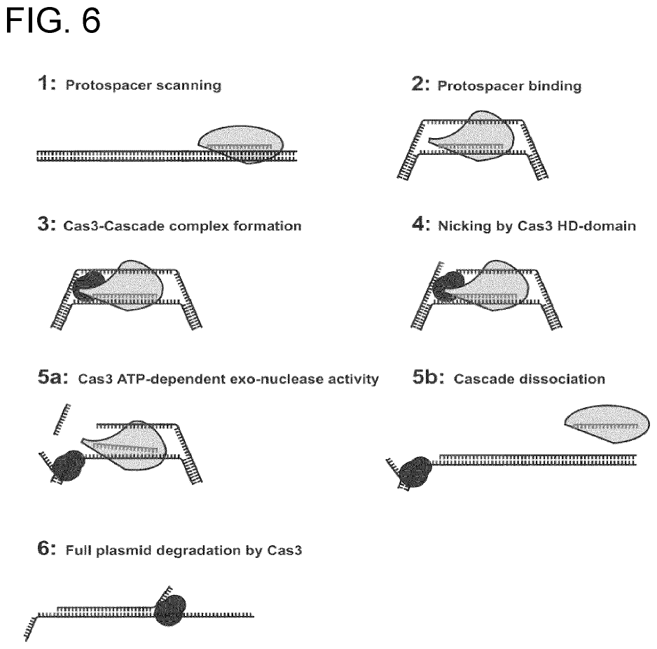

FIG. 6 is a schematic diagram showing a model of the CRISPR-interference type I pathway in E. coli.

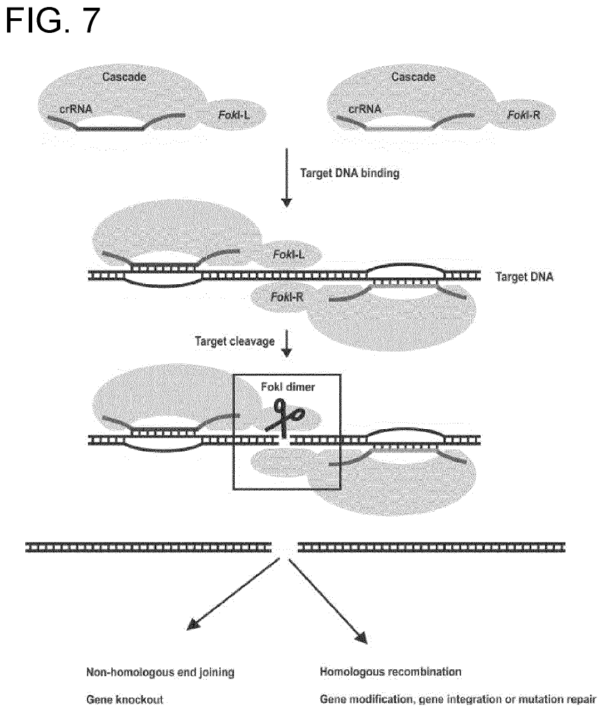

FIG. 7 is a schematic diagram showing how a Cascade-FokI fusion embodiment of the invention is used to create FokI dimers which cuts dsDNA to produce blunt ends as part of a process of non-homologous end joining or homologous recombination.

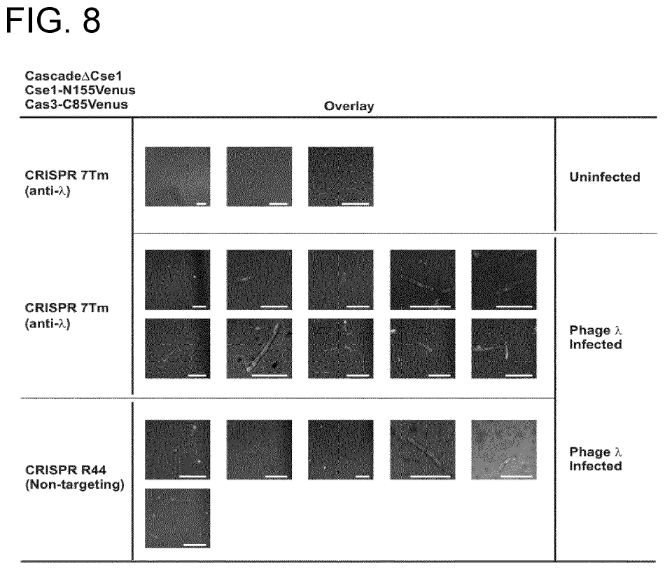

FIG. 8 shows how BiFC analysis reveals that Cascade and Cas3 interact upon target recognition. Overlay of Brightfield image and Venus fluorescence of cells expressing Cascade without Cse1, Cse1-N155Venus and Cas3-C85Venus and either CRISPR 7Tm, which targets 7 protospacers on the phage Lambda genome, or the non-targeting CRISPR R44. Cells expressing CRISPR 7Tm are fluorescent only when infected with phage Lambda, while cells expressing CRISPR R44 are non-fluorescent. The highly intense fluorescent dots (outside cells) are due to light-reflecting salt crystals. White bars correspond to 10 micron.

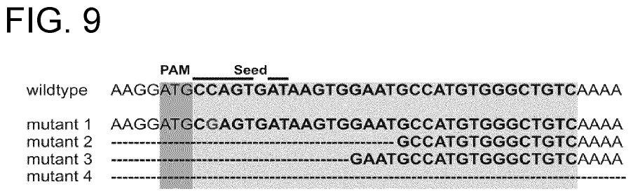

FIG. 9 shows pUC-λ sequences of 4 clones [SEQ ID NOs: 39-42] encoding CRISPR J3, Cascade and Cas3 (wt or S483AT485A) indicate that these are escape mutants carrying (partial) deletions of the protospacer or carrying a single point mutation in the seed region, which explains the inability to cure these plasmids.

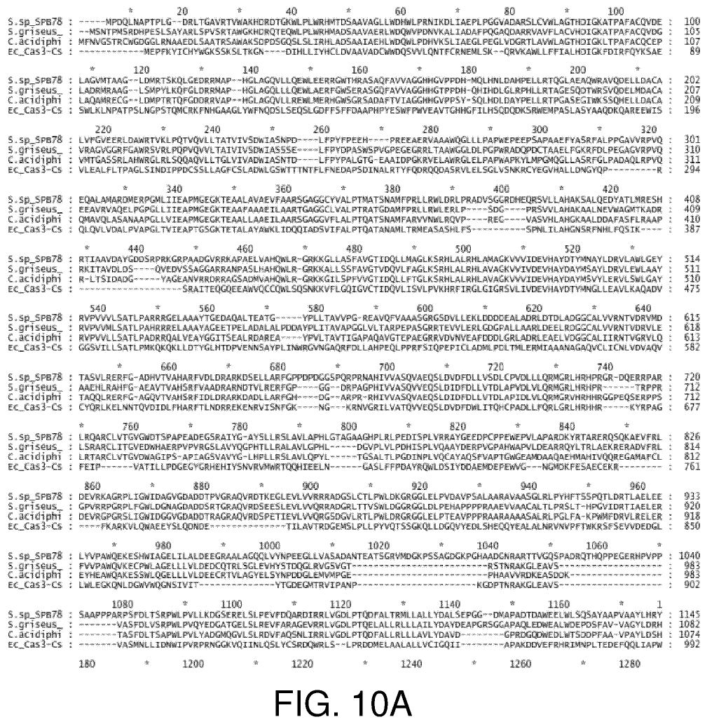

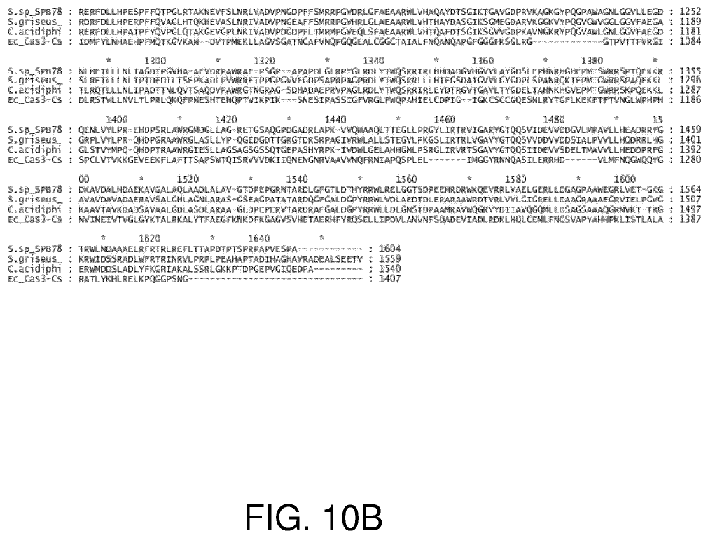

FIGS. 10A and 10B show sequence alignments of cas3 genes from organisms containing the Type I-E CRISPR/Cas system. Alignment of cas3-cse1 genes from Streptomyces sp. SPB78 (1^{st} sequence, Accession Number: ZP_07272643.1) [SEQ ID NO: 43], in Streptomyces griseus (2^{nd} sequence, Accession Number YP_001825054) [SEQ ID NO: 44], and in Catenulispora acidiphila DSM 44928 (3^{rd} sequence, Accession Number YP_003114638) [SEQ ID NO: 45] and an artificial E. coli Cas3-Cse1 fusion protein [SEQ ID NO: 46] which includes the polypeptide linker sequence from S. griseus.

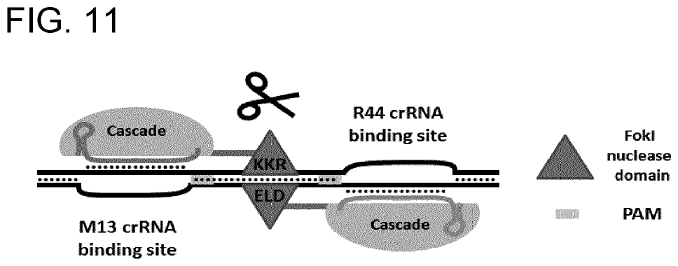

FIG. 11 shows the design of a Cascade^{KKR/ELD} nuclease pair in which FokI nuclease domains are mutated such that only heterodimers consisting of KKR and ELD nuclease domains are and the distance between the opposing binding sites may be varied to determine the optimal distance between a Cascade nuclease pair.

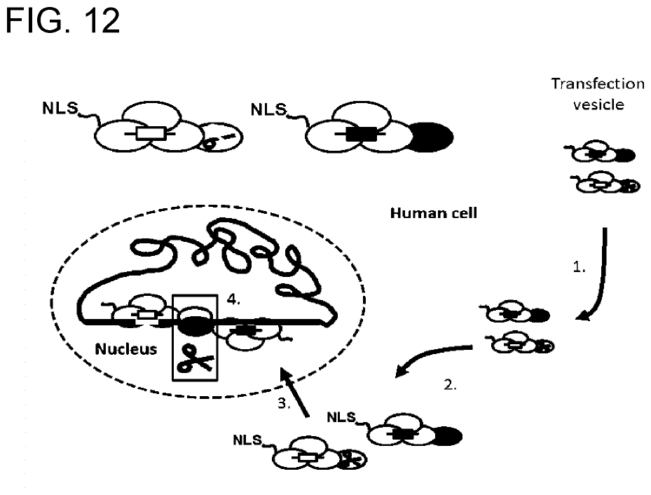

FIG. 12 is a schematic diagram showing genome targeting by a Cascade-FokI nuclease pair.

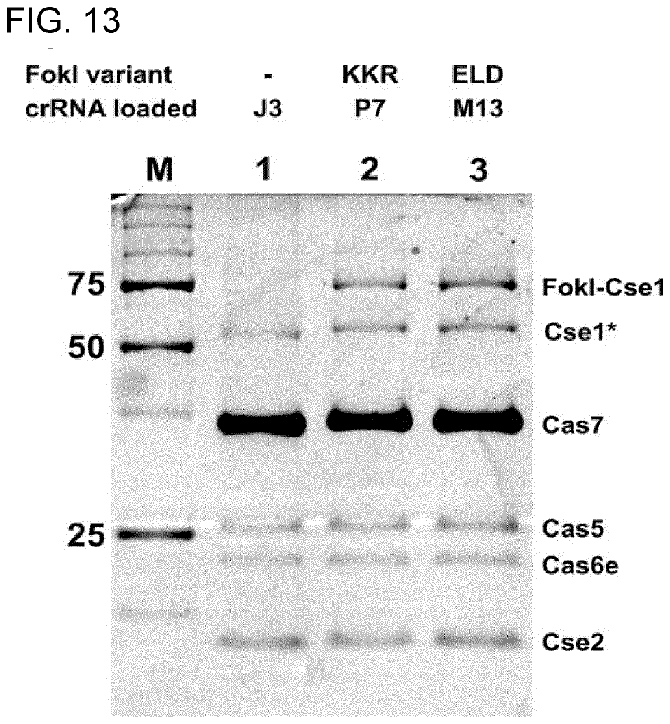

FIG. 13 shows an SDS PAGE gel of Cascade-nuclease complexes.

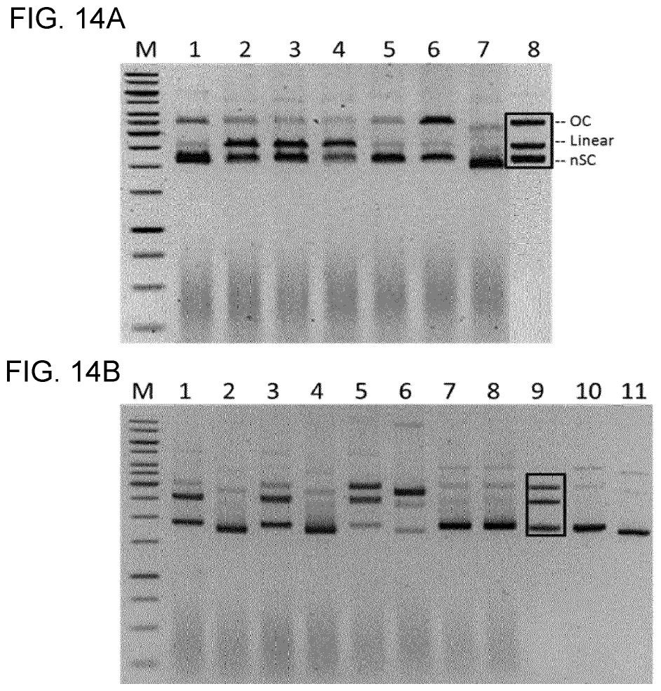

FIGS. 14A and 14B show electrophoresis gels of in vitro cleavage assays of Cascade^{KKR/ELD} on plasmid DNA.

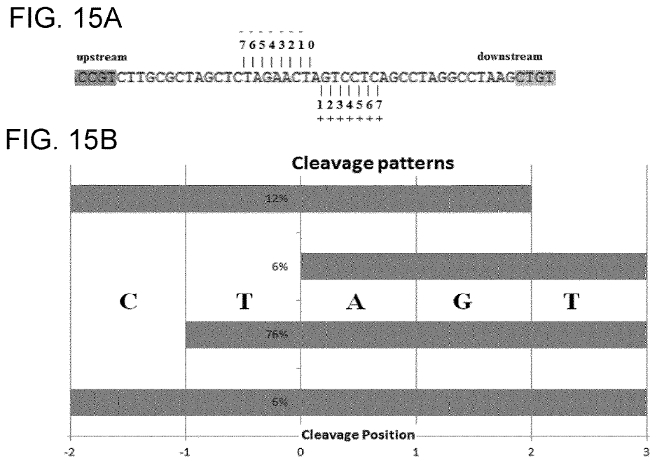

FIGS. 15 and 15B show Cascade^{KKR/ELD} cleavage patterns and frequency [SEQ ID NO: 47].

### EXAMPLES

### Materials and Methods Used

### Strains, Gene Cloning, Plasmids and Vectors

E. coli BL21-AI and E. coli BL21 (DE3) strains were used throughout. Table 1 lists all plasmids used in this study. The previously described pWUR408, pWUR480, pWUR404 and pWUR547 were used for production of Strep-tag II R44-Cascade, and pWUR408, pWUR514 and pWUR630 were used for production of Strep-tag II J3-Cascade (Jore et al., (2011) Nature Structural & Molecular Biology 18, 529-536; Semenova et al., (2011) Proceedings of the National Academy of Sciences of the United States of America 108, 10098-10103.) pUC-λ (pWUR610) and pUC-p7 (pWUR613) have been described elsewhere (Jore et al., 2011; Semenova et al., 2011). The C85Venus protein is encoded by pWUR647, which corresponds to pET52b (Novagen) containing the synthetic GA1070943 construct (Table 2) (Geneart) cloned between the BamHI and NotI sites. The N155Venus protein is encoded by pWUR648, which corresponds to pRSF1b (Novagen) containing the synthetic GA1070941 construct (Table 2) (Geneart) cloned between the NotI and XhoI sites. The Cas3-C85Venus fusion protein is encoded by pWUR649, which corresponds to pWUR647 containing the Cas3 amplification product using primers BG3186 and BG3213 (Table 3) between the NcoI and BamHI sites. The CasA-N155Venus fusion protein is encoded by pWUR650, which corresponds to pWUR648 containing the CasA amplification product using primers BG3303 and BG3212 (Table 3) between the NcoI and BamHI sites. CRISPR 7Tm is encoded by pWUR651, which corresponds to pACYCDuet-1 (Novagen) containing the synthetic GA1068859 construct (Table 2) (Geneart) cloned between the NcoI and KpnI sites. The Cascade encoding pWUR400, the CascadeΔCse1 encoding WUR401 and the Cas3 encoding pWUR397 were described previously (Jore et al., 2011). The Cas3H74A encoding pWUR652 was constructed using site directed mutagenesis of pWUR397 with primers BG3093, BG3094 (Table 3).

TABLE 1
Plasmids used

| | Description and order | Restriction | | |
|---|---|---|---|---|
| Plasmids | of genes (5′-3′) | sites | Primers | Source |
| pWUR397 | cas3 in pR5F-1b, no | | | 1 |
| | tags | | | |
| pWUR400 | casA-casB-casC-casD- | | | 1 |
| | casE in pCDF-1b, no | | | |
| | tags | | | |
| pWUR401 | casB-casC-casD-casE | | | 1 |
| | in pCDF-1b, no tags | | | |
| pWUR404 | casE in pCDF-1b, no | | | 1 |
| | tags | | | |
| pWUR408 | casA in pRSF-1b, no | | | 1 |
| | tags | | | |
| pWUR480 | casB with Strep-tag II | | | 1 |
| | (N-term)-casC-casD in | | | |
| | pET52b | | | |
| pWUR514 | casB with Strep-tag II | | | 2 |
| | (N-term)-casC-casD- | | | |
| | CasE in pET52b | | | |
| pWUR547 | E. coli R44 CRISPR, 7x | | | 2 |
| | spacer nr. 2, in | | | |
| | pACYCDuet-1 | | | |
| pWUR613 | pUC-p7; pUC19 | | | 2 |
| | containing R44- | | | |
| | protospacer on a 350 bp | | | |
| | phage P7 amplicon | | | |
| pWUR630 | CRISPR poly J3, 5x | NcoI/KpnI | | This |
| | spacer J3 in | | | study |
| | pACYCDuet-1 | | | |
| pWUR610 | pUC-λ; pUC19 | | | 3 |
| | containing J3- | | | |
| | protospacer on a 350 bp | | | |
| | phage λ amplicon | | | |
| pWUR647 | C85Venus; GA1070943 | BamHI/ | | This |
| | (Table S1) in pET52b | NotI | | study |
| pWUR648 | N155Venus; | NotI/XhoI | | This |
| | GA1070941 (Table S1) | | | study |
| | in pRSF 1b | | | |
| pWUR649 | cas3-C85Venus; | NcoI/ | BG3186 + | This |
| | pWUR647 containing | BamHI | BG3213 | study |
| | cas3 amplicon | | | |
| pWUR650 | casA-N155Venus | NcoI/NotI | BG3303 + | This |
| | pWUR648 containing | | BG3212 | study |
| | casA amplicon | | | |
| pWUR651 | CRISPR 7Tm; | NcoI/KpnI | | This |
| | GA1068859 (Table S1) | | | study |
| | in pACYCDuet-1 | | | |
| | casB with Strep-tag II | | | This |
| | (N-term)-casC-casD- | | | study |
| | CasE in pCDF-1b | | | |
| | cas3-casA fusion | | | This |
| | | | | study |
| | cas3H74A-CasA fusion | | | This |
| | | | | study |
| | cas3D75A-CasA fusion | | | This |
| | | | | study |
| | cas3K320N-CasA | | | This |
| | fusion | | | study |
| | cas3D452N-CasA | | | This |
| | fusion | | | study |

Source 1 in the table above is Brouns et al (2008) Science 321. 960-964.

Source 2 in the table above is Jore et al (2011) Nature Structural & Molecular Biology 18: 529-537.

TABLE 2
Synthetic Constructs

GA1070943
ACTGGAAAGCGGGCAGTGAAAGGAAGGCCCATGAGGCCAGTTAATTAAGC
GGATCCTGGCGGCGGCAGCGGCGGCGGCAGCGACAAGCAGAAGAACGGCA
TCAAGGCGAACTTCAAGATCCGCCACAACATCGAGGACGGCGGCGTGCAG
CTCGCCGACCACTACCAGCAGAACACCCCCATCGGCGACGGCCCCGTGCT
GCTGCCCGACAACCACTACCTGAGCTACCAGTCCGCCCTGAGCAAAGACC
CCAACGAGAAGCGCGATCACATGGTCCTGCTGGAGTTCGTGACCGCCGCC
GGGATCACTCTCGGCATGGACGAGCTGTACAAGTAAGCGGCCGCGGCGCG
CCTAGGCCTTGACGGCCTTCCTTCAATTCGCCCTATAGTGAG [SEQ ID
NO: 6]
GA1070941
CACTATAGGGCGAATTGGCGGAAGGCCGTCAAGGCCGCATTTAATTAAGC
GGCCGCAGGCGGCGGCAGCGGCGGCGGCAGCATGGTGAGCAAGGGCGAGG
AGCTGTTCACCGGGGTGGTGCCCATCCTGGTCGAGCTGGACGGCGACGTA
AACGGCCACAAGTTCAGCGTGTCCGGCGAGGGCGAGGGCGATGCCACCTA
CGGCAAGCTGACCCTGAAGCTCATCTGCACCACCGGCAAGCTGCCCGTGC
CCTGGCCCACCCTCGTGACCACCCTCGGCTACGGCCTGCAGTGCTTCGCC
CGCTACCCCGACCACATGAAGCAGCACGACTTCTTCAAGTCCGCCATGCC
CGAAGGCTACGTCCAGGAGCGCACCATCTTCTTCAAGGACGACGGCAACT
ACAAGACCCGCGCCGAGGTGAAGTTCGAGGGCGACACCCTGGTGAACCGC
ATCGAGCTGAAGGGCATCGACTTCAAGGAGGACGGCAACATCCTGGGGCA
CAAGCTGGAGTACAACTACAACAGCCACAACGTCTATATCACGGCCTAAC
TCGAGGGCGCGCCCTGGGCCTCATGGGCCTTCCGCTCACTGCCCGCTTTC
CAG [SEQ ID NO: 7]
GA1068859
CACTATAGGGCGAATTGGCGGAAGGCCGTCAAGGCCGCATGAGCTCCATG
GAAACAAAGAATTAGCTGATCTTTAATAATAAGGAAATGTTACATTAAGG
TTGGTGGGTTGTTTTTATGGGAAAAAATGCTTTAAGAACAAATGTATACT
TTTAGAGAGTTCCCCGCGCCAGCGGGGATAAACCGGGCCGATTGAAGGTC
CGGTGGATGGCTTAAAAGAGTTCCCCGCGCCAGCGGGGATAAACCGCCGC
AGGTACAGCAGGTAGCGCAGATCATCAAGAGTTCCCCGCGCCAGCGGGGA
TAAACCGACTTCTCTCCGAAAAGTCAGGACGCTGTGGCAGAGTTCCCCGC
GCCAGCGGGGATAAACCGCCTACGCGCTGAACGCCAGCGGTGTGGTGAAT
GAGTTCCCCGCGCCAGCGGGGATAAACCGGTGTGGCCATGCACGCCTTTA
ACGGTGAACTGGAGTTCCCCGCGCCAGCGGGGATAAACCGCACGAACTCA
GCCAGAACGACAAACAAAAGGCGAGTTCCCCGCGCCAGCGGGGATAAACC
GGCACCAGTACGCGCCCCACGCTGACGGTTTCTGAGTTCCCCGCGCCAGC
GGGGATAAACCGCAGCTCCCATTTTCAAACCCAGGTACCCTGGGCCTCAT
GGGCCTTCCGCTCACTGCCCGCTTTCCAG [SEQ ID NO: 8]
GA1047360
GAGCTCCCGGGCTGACGGTAATAGAGGCACCTACAGGCTCCGGTAAAACG
GAAACAGCGCTGGCCTATGCTTGGAAACTTATTGATCAACAAATTGCGGA
TAGTGTTATTTTTGCCCTCCCAACACAAGCTACCGCGAATGCTATGCTTA
CGAGAATGGAAGCGAGCGCGAGCCACTTATTTTCATCCCCAAATCTTATT
CTTGCTCATGGCAATTCACGGTTTAACCACCTCTTTCAATCAATAAAATC
ACGCGCGATTACTGAACAGGGGCAAGAAGAAGCGTGGGTTCAGTGTTGTC
AGTGGTTGTCACAAAGCAATAAGAAAGTGTTTCTTGGGCAAATCGGCGTT
TGCACGATTGATCAGGTGTTGATTTCGGTATTGCCAGTTAAACACCGCTT
TATCCGTGGTTTGGGAATTGGTAGATCTGTTTTAATTGTTAATGAAGTTC
ATGCTTACGACACCTATATGAACGGCTTGCTCGAGGCAGTGCTCAAGGCT
CAGGCTGATGTGGGAGGGAGTGTTATTCTTCTTTCCGCAACCCTACCAAT
GAAACAAAAACAGAAGCTTCTGGATACTTATGGTCTGCATACAGATCCAG
TGGAAAATAACTCCGCATATCCACTCATTAACTGGCGAGGTGTGAATGGT
GCGCAACGTTTTGATCTGCTAGCGGATCCGGTACC [SEQ ID NO: 9]

TABLE 3
Primers

| | |
|---|---|
| BG3186 | ATAGCGCCATGGAACCTTTTAAATATATATGCCATTA |
| | [SEQ ID NO: 10] |
| BG3213 | ACAGTGGGATCCGCTTTGGGATTTGCAGGGATGACTC |
| | TGGT [SEQ ID NO: 11] |
| BG3303 | ATAGCGTCATGAATTTGCTTATTGATAACTGGATTCC |
| | TGTACG [SEQ ID NO: 12] |
| BG3212 | ACAGTGGCGGCCGCGCCATTTGATGGCCCTCCTTGCG |
| | GTTTTAA [SEQ ID NO: 13] |
| BG3076 | CGTATATCAAACTTTCCAATAGCATGAAGAGCAATGA |
| | AAAATAAC [SEQ ID NO: 14] |
| BG3449 | ATGATACCGCGAGACCCACGCTC |
| | [SEQ ID NO: 15] |
| BG3451 | CGGATAAAGTTGCAGGACCACTTC |
| | [SEQ ID NO: 16] |

### Protein Production and Purification

Cascade was expressed and purified as described (Jore et al., 2011). Throughout purification a buffer containing 20 mM HEPES pH 7.5, 75 mM NaCl, 1 mM DTT, 2 mM EDTA was used for resuspension and washing. Protein elution was performed in the same buffer containing 4 mM desthiobiotin. The Cascade-Cas3 fusion complex was expressed and purified in the same manner, with washing steps being performed with 20 mM HEPES pH 7.5, 200 mM NaCl and 1 mM DTT, and elution in 20 mM HEPES pH 7.5, 75 mM NaCl, 1 mM DTT containing 4 mM desthiobiotin.

### Electrophoretic Mobility Shift Assay

Purified Cascade or Cascade subsomplexes were mixed with pUC-λ in a buffer containing 20 mM HEPES pH 7.5, 75 mM NaCl, 1 mM DTT, 2 mM EDTA, and incubated at 37° C. for 15 minutes. Samples were run overnight on a 0.8% TAE Agarose gel and post-stained with SybR safe (Invitrogen) 1:10000 dilution in TAE for 30 minutes. Cleavage with BsmI (Fermentas) or Nt.BspQI (New England Biolabs) was performed in the HEPES reaction buffer supplemented with 5 mM MgCl₂.

### Scanning Force Microscopy

Purified Cascade was mixed with pUC-λ (at a ratio of 7:1, 250 nM Cascade, 35 nM DNA) in a buffer containing 20 mM HEPES pH 7.5, 75 mM NaCl, 0.2 mM DTT, 0.3 mM EDTA and incubated at 37° C.for 15 minutes. Subsequently, for AFM sample preparation, the incubation mixture was diluted 10× in double distilled water and MgCl₂ was added at a final concentration of 1.2 mM. Deposition of the protein-DNA complexes and imaging was carried out as described before (Dame et al., (2000) Nucleic Acids Res. 28: 3504-3510).

### Fluorescence Microscopy

BL21-AI cells carrying CRISPR en cas gene encoding plasmids, were grown overnight at 37° C. in Luria-Bertani broth (LB) containing ampicillin (100 μg/ml), kanamycin (50 μg/ml), streptomycin (50 μg/ml) and chloramphenicol (34 μg/ml). Overnight culture was diluted 1:100 in fresh antibiotic-containing LB, and grown for 1 hour at 37° C. Expression of cas genes and CRISPR was induced for 1 hour by adding L-arabinose to a final concentration of 0.2% and IPTG to a final concentration of 1 mM. For infection, cells were mixed with phage Lambda at a Multiplicity of Infection (MOI) of 4. Cells were applied to poly-L-lysine covered microscope slides, and analyzed using a Zeiss LSM510 confocal laser scanning microscope based on an Axiovert inverted microscope, with a 40× oil immersion objective (N.A. of 1.3) and an argon laser as the excitation source (514 nm) and detection at 530-600 nm. The pinhole was set at 203 μm for all measurements.

pUC-λ Transformation Studies

LB containing kanamycin (50 μg/ml), streptomycin (50 μg/ml) and chloramphenicol (34 μg/ml) was inoculated from an overnight pre-inoculum and grown to an OD₆₀₀ of 0.3. Expression of cas genes and CRISPR was induced for 45 minutes with 0.2% L-arabinose and 1 mM IPTG. Cells were collected by centrifugation at 4° C. and made competent by resuspension in ice cold buffer containing 100 mM RbCl₂, 50 mM MnCl₂, 30 mM potassium acetate, 10 mM CaCl₂ and 15% glycerol, pH 5.8. After a 3 hour incubation, cells were collected and resuspended in a buffer containing 10 mM MOPS, 10 mM RbCI, 75 mM CaCl₂, 15% glycerol, pH 6.8. Transformation was performed by adding 80 ng pUC-λ, followed by a 1 minute heat-shock at 42° C., and 5 minute cold-shock on ice. Next cells were grown in LB for 45 minutes at 37° C. before plating on LB-agar plates containing 0.2% L-arabinose, 1 mM IPTG, ampicillin (100 μg/ml), kanamycin (50 μg/ml), streptomycin (50 μg/ml) and chloramphenicol (34 μg/ml).

Plasmid curing was analyzed by transforming BL21-AI cells containing cas gene and CRISPR encoding plasmids with pUC-λ, while growing the cells in the presence of 0.2% glucose to suppress expression of the T7-polymerase gene. Expression of cas genes and CRISPR was induced by collecting the cells and re-suspension in LB containing 0.2% arabinose and 1 mM IPTG. Cells were plated on LB-agar containing either streptomycin, kanamycin and chloramphenicol (non-selective for pUC-λ) or ampicillin, streptomycin, kanamycin and chloramphenicol (selective for pUC-λ). After overnight growth the percentage of plasmid loss can be calculated from the ratio of colony forming units on the selective and non-selective plates.

### Phage Lambda Infection Studies

Host sensitivity to phage infection was tested using a virulent phage Lambda (λᵥᵢᵣ), as in (Brouns et al (2008) Science 321, 960-964.). The sensitivity of the host to infection was calculated as the efficiency of plaquing (the plaque count ratio of a strain containing an anti-λ CRISPR to that of the strain containing a non-targeting R44 CRISPR) as described in Brouns et al (2008).

### Example 1

### Cascade Exclusively Binds Negatively Supercoiled Target DNA

The 3 kb pUC19-derived plasmid denoted pUC-λ, contains a 350 bp DNA fragment corresponding to part of the J gene of phage λ, which is targeted by J3-Cascade (Cascade associated with crRNA containing spacer J3 (Westra et al (2010) Molecular Microbiology 77, 1380-1393). The electrophoretic mobility shift assays show that Cascade has high affinity only for negatively supercoiled (nSC) target plasmid. At a molar ratio of J3-Cascade to pUC-λ of 6:1 all nSC plasmid was bound by Cascade, (see FIG. 1A), while Cascade carrying the non-targeting crRNA R44 (R44-Cascade) displayed non-specific binding at a molar ratio of 128:1 (see FIG. 1B). The dissociation constant (Kd) of nSC pUC-λ was determined to be 13±1.4 nM for J3-Cascade (see FIG. 1E) and 429±152 nM for R44-Cascade (see FIG. 1F).

J3-Cascade was unable to bind relaxed target DNA with measurable affinity, such as nicked (see FIG. 1C) or linear pUC-λ (see FIG. 1D), showing that Cascade has high affinity for larger DNA substrates with a nSC topology.

To distinguish non-specific binding from specific binding, the BsmI restriction site located within the protospacer was used. While adding BsmI enzyme to pUC-λ gives a linear product in the presence of R44-Cascade (see FIG. 1G, lane 4), pUC-λ is protected from BsmI cleavage in the presence of J3-Cascade (see FIG. 1G, lane 7), indicating specific binding to the protospacer. This shows that Cas3 is not required for in vitro sequence specific binding of Cascade to a protospacer sequence in a nSC plasmid.

Cascade binding to nSC pUC-λ was followed by nicking with Nt.BspQI, giving rise to an OC topology. Cascade is released from the plasmid after strand nicking, as can be seen from the absence of a mobility shift (see FIG. 1H, compare lane 8 to lane 10). In contrast, Cascade remains bound to its DNA target when a ssDNA probe complementary to the displaced strand is added to the reaction before DNA cleavage by Nt.BspQI (see FIG. 1H, lane 9). The probe artificially stabilizes the Cascade R-loop on relaxed target DNA. Similar observations are made when both DNA strands of pUC-λ are cleaved after Cascade binding (see FIG. 1I, lane 8 and lane 9).

### Example 2

### Cascade Induces Bending of Bound Target DNA

Complexes formed between purified Cascade and pUC-λ were visualized. Specific complexes containing a single bound J3-Cascade complex were formed, while unspecific R44-Cascade yields no DNA bound complexes in this assay under identical conditions. Out of 81 DNA molecules observed 76% were found to have J3-Cascade bound (see FIG. 2A-P). Of these complexes in most cases Cascade was found at the apex of a loop (86%), whereas a small fraction only was found at non-apical positions (14%). These data show that Cascade binding causes bending and possibly wrapping of the DNA, probably to facilitate local melting of the DNA duplex.

### Example 3

### Naturally Occurring Fusions of Cas3 and Cse1: Cas3 Interacts with Cascade Upon Protospacer Recognition

Figure S3 shows sequence analysis of cas3 genes from organisms containing the Type I-E CRISPR/Cas system reveals that Cas3 and Cse1 occur as fusion proteins in Streptomyces sp. SPB78 (Accession Number: ZP_07272643.1), in Streptomyces griseus (Accession Number YP_001825054), and in Catenulispora acidiphila DSM 44928 (Accession Number YP_003114638).

### Example 4

### Bimolecular Fluorescence Complementation (BiFC) Shows How a Cse1 Fusion Protein Forming Part of Cascade Continues to Interact With Cas3

BiFC experiments were used to monitor interactions between Cas3 and Cascade in vivo before and after phage λ infection. BiFC experiments rely on the capacity of the non-fluorescent halves of a fluorescent protein, e.g., Yellow Fluorescent Protein (YFP) to refold and to form a fluorescent molecule when the two halves occur in close proximity. As such, it provides a tool to reveal protein-protein interactions, since the efficiency of refolding is greatly enhanced if the local concentrations are high, e.g., when the two halves of the fluorescent protein are fused to interaction partners. Cse1 was fused at the C-terminus with the N-terminal 155 amino acids of Venus (Cse1-N155Venus), an improved version of YFP (Nagai et al (2002) Nature Biotechnology 20, 87-90). Cas3 was C-terminally fused to the C-terminal 85 amino acids of Venus (Cas3-C85Venus).

BiFC analysis reveals that Cascade does not interact with Cas3 in the absence of invading DNA (FIG. 3ABC, FIG. 3P and FIG. 8). Upon infection with phage λ, however, cells expressing CascadeΔCse1, Cse1-N155Venus and Cas3-C85Venus are fluorescent if they co-express the anti-λ CRISPR 7Tm (FIG. 3DEF, FIG. 3P and FIG. 8). When they co-express a non-targeting CRISPR R44 (FIG. 3GHI, FIG. 3P and FIG. 8), the cells remain non-fluorescent. This shows that Cascade and Cas3 specifically interact during infection upon protospacer recognition and that Cse1 and Cas3 are in close proximity of each other in the Cascade-Cas3 binary effector complex.

These results also show quite clearly that a fusion of Cse1 with an heterologous protein does not disrupt the ribonucleoprotein formation of Cascade and crRNA, nor does it disrupt the interaction of Cascade and Cas3 with the target phage DNA, even when the Cas3 itself is also a fusion protein.

### Example 5

### Preparing a Designed Cas3-Cse1 Fusion Gives a Protein With In Vivo Functional Activity

Providing in vitro evidence for Cas3 DNA cleavage activity required purified and active Cas3. Despite various solubilization strategies, Cas3 overproduced (Howard et al (2011) Biochem. J. 439, 85-95) in E. coli BL21 is mainly present in inactive aggregates and inclusion bodies. Cas3 was therefore produced as a Cas3-Cse1 fusion protein, containing a linker identical to that of the Cas3-Cse1 fusion protein in S. griseus (see FIG. 10). When co-expressed with CascadeΔCse1 and CRISPR J3, the fusion-complex was soluble and was obtained in high purity with the same apparent stoichiometry as Cascade (FIG. 5A). When functionality of this complex was tested for providing resistance against phage λ infection, the efficiency of plaquing (eop) on cells expressing the fusion-complex J3-Cascade-Cas3 was identical as on cells expressing the separate proteins (FIG. 5B).

Since the J3-Cascade-Cas3 fusion-complex was functional in vivo, in vitro DNA cleavage assays were carried out using this complex. When J3-Cascade-Cas3 was incubated with pUC-λ in the absence of divalent metals, plasmid binding was observed at molar ratios similar to those observed for Cascade (FIG. 5C), while a-specific binding to a non-target plasmid (pUC-p7, a pUC19 derived plasmid of the same size as pUC-λ, but lacking a protospacer) occurred only at high molar ratios (FIG. 5D), indicating that a-specific DNA binding of the complex is also similar to that of Cascade alone.

Interestingly, the J3-Cascade-Cas3 fusion complex displays magnesium dependent endonuclease activity on nSC target plasmids. In the presence of 10 mM Mg²⁺ J3-Cascade-Cas3 nicks nSC pUC-λ (FIG. 5E, lane 3-7), but no cleavage is observed for substrates that do not contain the target sequence (FIG. 5E, lane 9-13), or that have a relaxed topology. No shift of the resulting OC band is observed, in line with previous observations that Cascade dissociates spontaneously after cleavage, without requiring ATP-dependent Cas3 helicase activity. Instead, the helicase activity of Cas3 appears to be involved in exonucleolytic plasmid degradation. When both magnesium and ATP are added to the reaction, full plasmid degradation occurred (FIG. 5H).

The inventors have found that Cascade alone is unable to bind protospacers on relaxed DNA. In contrast, the inventors have found that Cascade efficiently locates targets in negatively supercoiled DNA, and subsequently recruits Cas3 via the Cse1 subunit. Endonucleolytic cleavage by the Cas3 HD-nuclease domain causes spontaneous release of Cascade from the DNA through the loss of supercoiling, remobilizing Cascade to locate new targets. The target is then progressively unwound and cleaved by the joint ATP-dependent helicase activity and HD-nuclease activity of Cas3, leading to complete target DNA degradation and neutralization of the invader.

Referring to FIG. 6 and without wishing to be bound to any particular theory, a mechanism of operation for the CRISPR-interference type I pathway in E. coli may involve (1) First, Cascade carrying a crRNA scans the nSC plasmid DNA for a protospacer, with adjacent PAM. Whether during this stage strand separation occurs is unknown. (2) Sequence specific protospacer binding is achieved through basepairing between the crRNA and the complementary strand of the DNA, forming an R-loop. Upon binding, Cascade induces bending of the DNA. (3) The Cse1 subunit of Cascade recruits Cas3 upon DNA binding. This may be achieved by Cascade conformational changes that take place upon nucleic acid binding. (4) The HD-domain (darker part) of Cas3 catalyzes Mg²⁺-dependent nicking of the displaced strand of the R-loop, thereby altering the topology of the target plasmid from nSC to relaxed OC. (5a and 5b) The plasmid relaxation causes spontaneous dissociation of Cascade. Meanwhile Cas3 displays ATP-dependent exonuclease activity on the target plasmid, requiring the helicase domain for target dsDNA unwinding and the HD-nuclease domain for successive cleavage activity. (6) Cas3 degrades the entire plasmid in an ATP-dependent manner as it processively moves along, unwinds and cleaves the target dsDNA.

### Example 6

### Preparation of Artificial Cas-Strep Tag Fusion Proteins and Assembly of Cascade Complexes

Cascade complexes are produced and purified as described in Brouns et al (2008) Science 321: 960-4 (2008), using the expression plasmids listed in Supplementary Table 3 of Jore et al (2011) Nature Structural & Molecular Biology 18: 529-537. Cascade is routinely purified with an N-terminal Strep-tag II fused to CasB (or CasC in CasCDE). Size exclusion chromatography (Superdex 200 HR 10/30 (GE)) is performed using 20 mM Tris-HCl (pH 8.0), 0.1 M NaCl, 1 mM dithiotreitol. Cascade preparations (˜0.3 mg) are incubated with DNase I (Invitrogen) in the presence of 2.5 mM MgCl₂ for 15 min at 37° C. prior to size exclusion analysis. Co-purified nucleic acids are isolated by extraction using an equal volume of phenol:chloroform:isoamylalcohol (25:24:1) pH 8.0 (Fluka), and incubated with either DNase I (Invitrogen) supplemented with 2.5 mM MgCl₂ or RNase A (Fermentas) for 10 min at 37° C. Cas subunit proteins fused to the amino acid sequence of Strep-Tag are produced.

Plaque assays showing the biological activity of the Strep-Tag Cascade subunits are performed using bacteriophage Lambda and the efficiency of plaquing (EOP) was calculated as described in Brouns et al (2008).

For purification of crRNA, samples are analyzed by ion-pair reversed-phased-HPLC on an Agilent 1100 HPLC with UV₂₆₀ₙₘ detector (Agilent) using a DNAsep column 50 mm×4.6 mm I. D. (Transgenomic, San Jose, CA). The chromatographic analysis is performed using the following buffer conditions: A) 0.1 M triethylammonium acetate (TEAA) (pH 7.0) (Fluka); B) buffer A with 25% LC MS grade acetonitrile (v/v) (Fisher). crRNA is obtained by injecting purified intact Cascade at 75° C. using a linear gradient starting at 15% buffer B and extending to 60% B in 12.5 min, followed by a linear extension to 100% B over 2 min at a flow rate of 1.0 ml/min. Hydrolysis of the cyclic phosphate terminus was performed by incubating the HPLC-purified crRNA in a final concentration of 0.1 M HCl at 4° C.for 1 hour. The samples are concentrated to 5-10 μl on a vacuum concentrator (Eppendorf) prior to ESI-MS analysis.

Electrospray Ionization Mass spectrometry analysis of crRNA is performed in negative mode using an UHR-TOF mass spectrometer (maXis) or an HCT Ultra PTM Discovery instrument (both Bruker Daltonics), coupled to an online capillary liquid chromatography system (Ultimate 3000, Dionex, UK). RNA separations are performed using a monolithic (PS-DVB) capillary column (200 μm×50 mm I.D., Dionex, UK). The chromatography is performed using the following buffer conditions: C) 0.4 M 1,1,1,3,3,3,-Hexafluoro-2-propanol (HFIP, Sigma-Aldrich) adjusted with tricthylamine (TEA) to pH 7.0 and 0.1 mM TEAA, and D) buffer C with 50% methanol (v/v) (Fisher). RNA analysis is performed at 50° ° C.with 20% buffer D, extending to 40% D in 5 min followed by a linear extension to 60% D over 8 min at a flow rate of 2 82 l/min.

Cascade protein is analyzed by native mass spectrometry in 0.15 M ammonium acetate (pH 8.0) at a protein concentration of 5 μM. The protein preparation is obtained by five sequential concentration and dilution steps at 4° C.using a centrifugal filter with a cut-off of 10 kDa (Millipore). Proteins are sprayed from borosilicate glass capillaries and analyzed on a LCT electrospray time-of-flight or modified quadrupole time-of-flight instruments (both Waters, UK) adjusted for optimal performance in high mass detection (see Tahallah N et al (2001) Rapid Commun Mass Spectrom 15: 596-601 (2001) and van den Heuvel, R.H. et al. Anal Chem 78: 7473-83 (2006). Exact mass measurements of the individual Cas proteins were acquired under denaturing conditions (50% acetonitrile, 50% MQ, 0.1% formic acid). Sub-complexes in solution were generated by the addition of 2-propanol to the spray solution to a final concentration of 5% (v/v). Instrument settings were as follows; needle voltage ˜1.2 kV, cone voltage ˜175 V, source pressure 9 mbar. Xenon was used as the collision gas for tandem mass spectrometric analysis at a pressure of 1.5 10⁻² mbar. The collision voltage varied between 10-200 V.

Electrophoretic mobility shift assays (EMSA) are used to demonstrate the functional activity of Cascade complexes for target nucleic acids. EMSA is performed by incubating Cascade, CasBCDE or CasCDE with 1 nM labelled nucleic acid in 50 mM Tris-Cl pH 7.5, 100 mM NaCl. Salmon sperm DNA (Invitrogen) is used as competitor. EMSA reactions are incubated at 37° C.for 20-30 min prior to electrophoresis on 5% polyacrylamide gels. The gels are dried and analyzed using phosphor storage screens and a PMI phosphor imager (Bio-Rad). Target DNA binding and cleavage activity of Cascade is tested in the presence of 1-10 mM Ca, Mg or Mn-ions.

DNA targets are gel-purified long oligonucleotides (Isogen Life Sciences or Biolegio), listed in Supplementary Table 3 of Jore et al (2011). The oligonucleotides are end-labeled using γ³²P-ATP (PerkinElmer) and T4 kinase (Fermentas). Double-stranded DNA targets are prepared by annealing complementary oligonucleotides and digesting remaining ssDNA with Exonuclease I (Fermentas). Labelled RNA targets are in vitro transcribed using T7 Maxiscript or T7 Mega Shortscript kits (Ambion) with α³²P-CTP (PerkinElmer) and removing template by DNase I (Fermentas) digestion. Double stranded RNA targets are prepared by annealing complementary RNAs and digesting surplus ssRNA with RNase TI (Fermentas), followed by phenol extraction.

Plasmid mobility shift assays are performed using plasmid pWUR613 containing the R44 protospacer. The fragment containing the protospacer is PCR-amplified from bacteriophage P7 genomic DNA using primers BG3297 and BG 3298 (see Supplementary Table 3 of Jore et al (2011). Plasmid (0.4 μg) and Cascade were mixed in a 1:10 molar ratio in a buffer containing 5 mM Tris-HCl (pH 7.5) and 20 mM NaCl and incubated at 37° C.for 30 minutes. Cascade proteins were then removed by proteinase K treatment (Fluka) (0.15 U, 15 min, 37° C.) followed by phenol/chloroform extraction. RNA-DNA complexes were then treated with RNaseH (Promega) (2 U, 1 h, 37° C.).

Strep-Tag-Cas protein subunit fusions which form Cascade protein complexes or active sub-complexes with the RNA component (equivalent to a crRNA), have the expected biological and functional activity of scanning and specific attachment and cleavage of nucleic acid targets. Fusions of the Cas subunits with the amino acid chains of fluorescent dyes also form Cascade complexes and sub-complexes with the RNA component (equivalent to crRNA) which retains biological and functional activity and allows visualisation of the location of a target nucleic acid sequence in ds DNA for example.

### Example 7

### A Cascade-Nuclease Pair and Test of Nuclease Activity In Vitro

Six mutations designated “Sharkey” have been introduced by random mutagenesis and screening to improve nuclease activity and stability of the non-specific nuclease domain from Flavobacterium okeanokoites restriction enzyme FokI (see Guo, J., et al. (2010) J. Mol. Biol. 400: 96-107). Other mutations have been introduced that reduce off-target cleavage activity. This is achieved by engineering electrostatic interactions at the FokI dimer interface of a ZFN pair, creating one FokI variant with a positively charged interface (KKR, E490K, I538K, H537R) and another with a negatively charged interface (ELD, Q486E, 1499L, N496D) (see Doyon, Y., et al., (2011) Nature Methods 8: 74-9). Each of these variants is catalytically inactive as a homodimer, thereby reducing the frequency of off-target cleavage.

### Cascade-Nuclease Design

We translationally fused improved FokI nucleases to the N-terminus of Cse1 to generate variants of Cse1 being FokI^{KKR}-Cse1 and FokI^{ELD}-Cse1, respectively. These two variants are co-expressed with Cascade subunits (Cse2, Cas7, Cas5 and Cas6e), and one of two distinct CRISPR plasmids with uniform spacers. This loads the Cascade^{KKR} complex with uniform P7-crRNA, and the Cascade^{ELD} complex with uniform M13 g8-crRNA. These complexes are purified using the N-terminally StrepII-tagged Cse2 as described in Jore, M.M., et al., (2011) Nat. Struct. Mol. Biol. 18(5): 529-536. Furthermore an additional purification step can be carried out using an N-terminally HIS-tagged FokI, to ensure purifying full length and intact Cascade-nuclease fusion complexes.

The nucleotide and amino acid sequences of the fusion proteins used in this example were as follows:

>nucleotide sequence of FokI-(Sharkey-ELD)-Cse1
[SEQ ID NO: 18]
ATGGCTCAACTGGTTAAAAGCGAACTGGAAGAGAAAAAAAGTGAACTGCGCCACAAACTGAAA
TATGTGCCGCATGAATATATCGAGCTGATTGAAATTGCACGTAATCCGACCCAGGATCGTATT
CTGGAAATGAAAGTGATGGAATTTTTTATGAAAGTGTACGGCTATCGCGGTGAACATCTGGGT
GGTAGCCGTAAACCGGATGGTGCAATTTATACCGTTGGTAGCCCGATTGATTATGGTGTTATT
GTTGATACCAAAGCCTATAGCGGTGGTTATAATCTGCCGATTGGTCAGGCAGATGAAATGGAA
CGTTATGTGGAAGAAAATCAGACCCGTGATAAACATCTGAATCCGAATGAATGGTGGAAAGTT
TATCCGAGCAGCGTTACCGAGTTTAAATTCCTGTTTGTTAGCGGTCACTTCAAAGGCAACTAT
AAAGCACAGCTGACCCGTCTGAATCATATTACCAATTGTAATGGTGCAGTTCTGAGCGTTGAA
GAACTGCTGATTGGTGGTGAAATGATTAAAGCAGGCACCCTGACCCTGGAAGAAGTTCGTCGC
AAATTTAACAATGGCGAAATCAACTTTGCGGATCCCACCAACCGCGCGAAAGGCCTGGAAGCG
GTGAGCGTGGCGAGCatgaatttgcttattgataactggattcctgtacgcccgcgaaacggg
gggaaagtccaaatcataaatctgcaatcgctatactgcagtagagatcagtggcgattaagt
ttgccccgtgacgatatggaactggccgctttagcactgctggtttgcattgggcaaattatc
gccccggcaaaagatgacgttgaatttcgacatcgcataatgaatccgctcactgaagatgag
tttcaacaactcatcgcgccgtggatagatatgttctaccttaatcacgcagaacatccatta
tgcagaccaaaggtgtcaaagcaaatgatgtgactccaatggaaaaactgttggctggggtaa
gcggcgcgacgaattgtgcatttgtcaatcaaccggggcagggtgaagcattatgtggtggat
gcactgcgattgcgttattcaaccaggcgaatcaggcaccaggtfttggtggtggtfttaaaa
gcggtttacgtggaggaacacctgtaacaacgttcgtacgtgggatcgatcttcgttcaacgg
tgttactcaatgtcctcacattacctcgtcttcaaaaacaatttcctaatgaatcacatacgg
aaaaccaacctacctggattaaacctatcaagtccaatgagtctatacctgatcgtcaattgg
gtftgtccgtggtctattctggcaaccagcgcatattgaattatgcgatcccattgggattgg
taaatgttatgctgtggacaggaaagcaatttgcgttataccggttftcttaaggaaaaattt
acctttacagttaatgggctatggccccatccgcattcccatgtctggtaacagtcaagaaag
gggaggttgaggaaaaatttcttgctttcaccacctccgcaccatcatggacacaaatcagcc
gagttgtggtagataagattattcaaaatgaaaatggaaatcgcgtggcggcggttgtgaatc
aattcagaaatattgcgccgcaaagtcctcttgaattgattatggggggatatcgtaataatc
aagcatctattcttgaacggcgtcatgatgtgttgatgtttaatcaggggtggcaacaatacg
gcaatgtgataaacgaaatagtgactgttggtttgggatataaaacagccttacgcaaggcgt
tatatacctttgcagaagggfttaaaaataaagacttcaaaggggccggagtctctgttcatg
agactgcagaaaggcatttctatcgacagagtgaattattaattcccgatgtactggcgaatg
ttaatttttcccaggctgatgaggtaatagctgatttacgagacaaacttcatcaattgtgtg
aaatgctatttaatcaatctgtagctccctatgcacatcatcctaaattaataagcacattag
cgcttgcccgcgccacgctatacaaacatttacgggagttaaaaccgcaaggagggccatcaa
atggctga
>protein sequence of FokI-(Sharkey-ELD)-Cse1
[SEQ ID NO: 19]
MAQLVKSELEEKKSELRHKLKYVPHEYIELIEIARNPTQDRILEMKVMEFFMKVYGYRGEHLG
GSRKPDGAIYTVGSPIDYGVIVDTKAYSGGYNLPIGQADEMERYVEENQTRDKHLNPNEWWKV
YPSSVTEFKFLFVSGHFKGNYKAQLTRLNHITNCNGAVLSVEELLIGGEMIKAGTLTLEEVRR
KFNNGEINFADPTNRAKGLEAVSVASMNLLIDNWIPVRPRNGGKVQIINLQSLYCSRDQWRLS
LPRDDMELAALALLVCIGQIIAPAKDDVEFRHRIMNPLTEDEFQQLIAPWIDMFYLNHAEHPF
MQTKGVKANDVTPMEKLLAGVSGATNCAFVNQPGQGEALCGGCTAIALFNQANQAPGFGGGFK
SGLRGGTPVTTFVRGIDLRSTVLLNVLTLPRLQKQFPNESHTENQPTWIKPIKSNESIPASSI
GFVRGLFWQPAHIELCDPIGIGKCSCCGQESNLRYTGFLKEKFTFTVNGLWPHPHSPCLVTVK
KGEVEEKFLAFTTSAPSWTQISRVVVDKIIQNENGNRVAAVVNQFRNIAPQSPLELIMGGYRN
NQASILERRHDVL1VIENQGWQQYGNVINEIVTVGLGYKTALRKALYTFAEGFKNKDFKGAGV
SVHETAERHFYRQSELLIPDVLANVNFSQADEVIADLRDKLHQLCEMLFNQSVAPYAHHPKLI
STLALARATLYKHLRELKPQGGPSNG*
>nucleotide sequence of FokI-(Sharkey-KKR)-Cse1
[SEQ ID NO: 20]
ATGGCTCAACTGGTTAAAAGCGAACTGGAAGAGAAAAAAAGTGAACTGCGCCACAAACTGAAA
TATGTGCCGCATGAATATATCGAGCTGATTGAAATTGCACGTAATCCGACCCAGGATCGTATT
CTGGAAATGAAAGTGATGGAATTTTTTATGAAAGTGTACGGCTATCGCGGTGAACATCTGGGT
GGTAGCCGTAAACCGGATGGTGCAATTTATACCGTTGGTAGCCCGATTGATTATGGTGTTATT
GTTGATACCAAAGCCTATAGCGGTGGTTATAATCTGCCGATTGGTCAGGCAGATGAAATGCAG
CGTTATGTGAAAGAAAATCAGACCCGCAACAAACATATTAACCCGAATGAATGGTGGAAAGTT
TATCCGAGCAGCGTTACCGAGTTTAAATTCCTGTTTGTTAGCGGTCACTTCAAAGGCAACTAT
AAAGCACAGCTGACCCGTCTGAATCGTAAAACCAATTGTAATGGTGCAGTTCTGAGCGTTGAA
GAACTGCTGATTGGTGGTGAAATGATTAAAGCAGGCACCCTGACCCTGGAAGAAGTTCGTCGC
AAATTTAACAATGGCGAAATCAACTTTGCGGATCCCACCAACCGCGCGAAAGGCCTGGAAGCG
GTGAGCGTGGCGAGCatgaatttgcttattgataactggattcctgtacgcccgcgaaacggg
gggaaagtccaaatcataaatctgcaatcgctatactgcagtagagatcagtggcgattaagt
ttgccccgtgacgatatggaactggccgctttagcactgctggtttgcattgggcaaattatc
gccccggcaaaagatgacgttgaatttcgacatcgcataatgaatccgctcactgaagatgag
tttcaacaactcatcgcgccgtggatagatatgttctaccttaatcacgcagaacatccatta
tgcagaccaaaggtgtcaaagcaaatgatgtgactccaatggaaaaactgttggctggggtaa
gcggcgcgacgaattgtgcatttgtcaatcaaccggggcagggtgaagcattatgtggtggat
gcactgcgattgcgttattcaaccaggcgaatcaggcaccaggttftggtggtggttttaaaa
gcggtttacgtggaggaacacctgtaacaacgttcgtacgtgggatcgatcttcgttcaacgg
tgttactcaatgtcctcacattacctcgtcttcaaaaacaatttcctaatgaatcacatacgg
aaaaccaacctacctggattaaacctatcaagtccaatgagtctatacctgcttcgtcaattg
ggtttgtccgtggtctattctggcaaccagcgcatattgaattatgcgatcccattgggattg
gtaaatgttcttgctgtggacaggaaagcaatttgcgttataccggttttcttaaggaaaaat
ttacctttacagttaatgggctatggccccatccgcattccccttgtctggtaacagtcaaga
aaggggaggttgaggaaaaatttcttgctttcaccacctccgcaccatcatggacacaaatca
gccgagttgtggtagataagattattcaaaatgaaaatggaaatcgcgtggcggcggttgtga
atcaattcagaaatattgcgccgcaaagtcctcttgaattgattatggggggatatcgtaata
atcaagcatctattcttgaacggcgtcatgatgtgttgatgtttaatcaggggtggcaacaat
acggcaatgtgataaacgaaatagtgactgttggtttgggatataaaacagccttacgcaagg
cgttatatacctttgcagaagggtttaaaaataaagacttcaaaggggccggagtctctgttc
atgagactgcagaaaggcatttctatcgacagagtgaattattaattcccgatgtactggcga
atgttaatttttcccaggctgatgaggtaatagctgatttacgagacaaacttcatcaattgt
gtgaaatgctatttaatcaatctgtagctccctatgcacatcatcctaaattaataagcacat
tagcgcttgcccgcgccacgctatacaaacatttacgggagttaaaaccgcaaggagggccat
caaatggctga
>protein sequence of FokI-(Sharkey-KKR)-Cse1
[SEQ ID NO: 21]
MAQLVKSELEEKKSELRHKLKYVPHEYIELIEIARNPTQDRILEMKVMEFFMKVYGYRGEHLG
GSRKPDGAIYTVGSPIDYGVIVDTKAYSGGYNLPIGQADEMQRYVKENQTRNKHINPNEWWKV
YPSSVTEFKFLFVSGHFKGNYKAQLTRLNRKTNCNGAVLSVEELLIGGEMIKAGTLTLEEVRR
KFNNGEINFADPTNRAKGLEAVSVASMNLLIDNWIPVRPRNGGKVQIINLQSLYCSRDQWRLS
LPRDDMELAALALLVCIGQIIAPAKDDVEFRHRIMNPLTEDEFQQLIAPWIDMFYLNHAEHPF
MQTKGVKANDVTPMEKLLAGVSGATNCAFVNQPGQGEALCGGCTAIALFNQANQAPGFGGGFK
SGLRGGTPVTTFVRGIDLRSTVLLNVLTLPRLQKQFPNESHTENQPTWIKPIKSNESIPASSI
GFVRGLFWQPAHIELCDPIGIGKCSCCGQESNLRYTGFLKEKFTFTVNGLWPHPHSPCLVTVK
KGEVEEKFLAFTTSAPSWTQISRVVVDKIIQNENGNRVAAVVNQFRNIAPQSPLELEVIGGYR
NNQASILERRHDVL1VIENQGWQQYGNVINEIVTVGLGYKTALRKALYTFAEGFKNKDFKGAG
VSVHETAERHFYRQSELLIPDVLANVNFSQADEVIADLRDKLHQLCEMLFNQSVAPYAHHPKL
ISTLALARATLYKHLRELKPQGGPSNG*
>nuclotide sequence of His₆-Dual-monopartite NLS
SV40-FokI-(Sharkey-KKR)-Cse1
[SEQ ID NO: 22]
ATGcatcaccatcatcaccacCCGAAAAAAAAGCGCAAAGTGGATCCGAAGAAAAAACGTAAA
GTTGAAGATCCGAAAGACATGGCTCAACTGGTTAAAAGCGAACTGGAAGAGAAAAAAAGTGAA
CTGCGCCACAAACTGAAATATGTGCCGCATGAATATATCGAGCTGATTGAAATTGCACGTAAT
CCGACCCAGGATCGTATTCTGGAAATGAAAGTGATGGAATTTTTTATGAAAGTGTACGGCTAT
CGCGGTGAACATCTGGGTGGTAGCCGTAAACCGGATGGTGCAATTTATACCGTTGGTAGCCCG
ATTGATTATGGTGTTATTGTTGATACCAAAGCCTATAGCGGTGGTTATAATCTGCCGATTGGT
CAGGCAGATGAAATGCAGCGTTATGTGAAAGAAAATCAGACCCGCAACAAACATATTAACCCG
AATGAATGGTGGAAAGTTTATCCGAGCAGCGTTACCGAGTTTAAATTCCTGTTTGTTAGCGGT
CACTTCAAAGGCAACTATAAAGCACAGCTGACCCGTCTGAATCGTAAAACCAATTGTAATGGT
GCAGTTCTGAGCGTTGAAGAACTGCTGATTGGTGGTGAAATGATTAAAGCAGGCACCCTGACC
CTGGAAGAAGTTCGTCGCAAATTTAACAATGGCGAAATCAACTTTGCGGATCCCACCAACCGC
GCGAAAGGCCTGGAAGCGGTGAGCGTGGCGAGCatgaatttgcttattgataactggattcct
gtacgcccgcgaaacggggggaaagtccaaatcataaatctgcaatcgctatactgcagtaga
gatcagtggcgattaagtttgccccgtgacgatatggaactggccgctttagcactgctggtt
tgcattgggcaaattatcgccccggcaaaagatgacgttgaatttcgacatcgcataatgaat
ccgctcactgaagatgagtttcaacaactcatcgcgccgtggatagatatgttctaccttaat
cacgcagaacatccctttatgcagaccaaaggtgtcaaagcaaatgatgtgactccaatggaa
aaactgttggctggggtaagcggcgcgacgaattgtgcatttgtcaatcaaccggggcagggt
gaagcattatgtggtggatgcactgcgattgcgttattcaaccaggcgaatcaggcaccaggt
tttggtggtggttttaaaagcggtttacgtggaggaacacctgtaacaacgttcgtacgtggg
atcgatcttcgttcaacggtgttactcaatgtcctcacattacctcgtcttcaaaaacaattt
cctaatgaatcacatacggaaaaccaacctacctggattaaacctatcaagtccaatgagtct
atacctgcttcgtcaattgggtttgtccgtggtctattctggcaaccagcgcatattgaatta
tgcgatcccattgggattggtaaatgttcttgctgtggacaggaaagcaatttgcgttatacc
ggttftcttaaggaaaaatttacctttacagttaatgggctatggccccatccgcattcccct
tgtctggtaacagtcaagaaaggggaggttgaggaaaaatttcttgctttcaccacctccgca
ccatcatggacacaaatcagccgagttgtggtagataagattattcaaaatgaaaatggaaat
cgcgtggcggcggttgtgaatcaattcagaaatattgcgccgcaaagtcctcttgaattgatt
atggggggatatcgtaataatcaagcatctattatgaacggcgtcatgatgtgttgatgttta
atcaggggtggcaacaatacggcaatgtgataaacgaaatagtgactgttggtttgggatata
aaacagccttacgcaaggcgttatatacctttgcagaagggtttaaaaataaagacttcaaag
gggccggagtctctgttcatgagactgcagaaaggcatttctatcgacagagtgaattattaa
ttcccgatgtactggcgaatgttaatttttcccaggctgatgaggtaatagctgatttacgag
acaaacttcatcaattgtgtgaaatgctatttaatcaatctgtagctccctatgcacatcatc
ctaaattaataagcacattagcgcttgcccgcgccacgctatacaaacatttacgggagttaa
aaccgcaaggagggccatcaaatggctga
>protein sequence of His₆-Dual-monopartite NLS
SV40-FokI-(Sharkey-KKR)-Cse1
[SEQ ID NO: 23]
MHHHHHHPKKKRKVDPKKKRKVEDPKDMAQLVKSELEEKKSELRHKLKYVPHEYIELIEIARN
PTQDRILEMKVMEFFMKVYGYRGEHLGGSRKPDGAIYTVGSPIDYGVIVDTKAYSGGYNLPIG
QADEMQRYVKENQTRNKHINPNEWWKVYPSSVTEFKFLFVSGHFKGNYKAQLTRLNRKTNCNG
AVLSVEELLIGGEMIKAGTLTLEEVRRKFNNGEINFADPTNRAKGLEAVSVASMNLLIDNWIP
VRPRNGGKVQIINLQSLYCSRDQWRLSLPRDDMELAALALLVCIGQIIAPAKDDVEFRHRIMN
PLTEDEFQQLIAPWID1VIFYLNHAEHPFMQTKGVKANDVTPMEKLLAGVSGATNCAFVNQPG
QGEALCGGCTAIALFNQANQAPGFGGGFKSGLRGGTPVTTFVRGIDLRSTVLLNVLTLPRLQK
QFPNESHTENQPTWIKPIKSNESIPASSIGFVRGLFWQPAHIELCDPIGIGKCSCCGQESNLR
YTGFLKEKFTFTVNGLWPHPHSPCLVTVKKGEVEEKFLAFTTSAPSWTQISRVVVDKIIQNEN
GNRVAAVVNQFRNIAPQSPLELIMGGYRNNQASILERRHDVLMFNQGWQQYGNVINEIVTVGL
GYKTALRKALYTFAEGFKNKDFKGAGVSVHETAERHFYRQSELLIPDVLANVNFSQADEVIAD
LRDKLHQLCEMLFNQSVAPYAHHPKLISTLALARATLYKHLRELKPQGGPSNG*
>nucleotide sequence of His₆-Dual-monopartite NLS
SV40-FokI (Sharkey-ELD)-Cse1
[SEQ ID NO: 24]
ATGcatcaccatcatcaccacCCGAAAAAAAAGCGCAAAGTGGATCCGAAGAAAAAACGTAAA
GTTGAAGATCCGAAAGACATGGCTCAACTGGTTAAAAGCGAACTGGAAGAGAAAAAAAGTGAA
CTGCGCCACAAACTGAAATATGTGCCGCATGAATATATCGAGCTGATTGAAATTGCACGTAAT
CCGACCCAGGATCGTATTCTGGAAATGAAAGTGATGGAATTTTTTATGAAAGTGTACGGCTAT
CGCGGTGAACATCTGGGTGGTAGCCGTAAACCGGATGGTGCAATTTATACCGTTGGTAGCCCG
ATTGATTATGGTGTTATTGTTGATACCAAAGCCTATAGCGGTGGTTATAATCTGCCGATTGGT
CAGGCAGATGAAATGGAACGTTATGTGGAAGAAAATCAGACCCGTGATAAACATCTGAATCCG
AATGAATGGTGGAAAGTTTATCCGAGCAGCGTTACCGAGTTTAAATTCCTGTTTGTTAGCGGT
CACTTCAAAGGCAACTATAAAGCACAGCTGACCCGTCTGAATCATATTACCAATTGTAATGGT
GCAGTTCTGAGCGTTGAAGAACTGCTGATTGGTGGTGAAATGATTAAAGCAGGCACCCTGACC
CTGGAAGAAGTTCGTCGCAAATTTAACAATGGCGAAATCAACTTTGCGGATCCCACCAACCGC
GCGAAAGGCCTGGAAGCGGTGAGCGTGGCGAGCatgaatttgcttattgataactggattcct
gtacgcccgcgaaacggggggaaagtccaaatcataaatctgcaatcgctatactgcagtaga
gatcagtggcgattaagtttgccccgtgacgatatggaactggccgctttagcactgctggtt
tgcattgggcaaattatcgccccggcaaaagatgacgttgaatttcgacatcgcataatgaat
ccgctcactgaagatgagtttcaacaactcatcgcgccgtggatagatatgttctaccttaat
cacgcagaacatccctttatgcagaccaaaggtgtcaaagcaaatgatgtgactccaatggaa
aaactgttggctggggtaagcggcgcgacgaattgtgcatttgtcaatcaaccggggcagggt
gaagcattatgtggtggatgcactgcgattgcgttattcaaccaggcgaatcaggcaccaggt
tttggtggtggttttaaaagcggtttacgtggaggaacacctgtaacaacgttcgtacgtggg
atcgatcttcgttcaacggtgttactcaatgtcctcacattacctcgtcttcaaaaacaattt
cctaatgaatcacatacggaaaaccaacctacctggattaaacctatcaagtccaatgagtct
atacctgcttcgtcaattgggtttgtccgtggtctattctggcaaccagcgcatattgaatta
tgcgatcccattgggattggtaaatgttcttgctgtggacaggaaagcaatttgcgttatacc
ggttttcttaaggaaaaatttacctttacagttaatgggctatggccccatccgcattcccct
tgtctggtaacagtcaagaaaggggaggttgaggaaaaatttcttgctttcaccacctccgca
ccatcatggacacaaatcagccgagttgtggtagataagattattcaaaatgaaaatggaaat
cgcgtggcggcggttgtgaatcaattcagaaatattgcgccgcaaagtcctcttgaattgatt
atggggggatatcgtaataatcaagcatctattcttgaacggcgtcatgatgtgttgatgttt
aatcaggggtggcaacaatacggcaatgtgataaacgaaatagtgactgttggtttgggatat
aaaacagccttacgcaaggcgttatatacctttgcagaagggtttaaaaataaagacttcaaa
ggggccggagtctctgttcatgagactgcagaaaggcatttctatcgacagagtgaattatta
attcccgatgtactggcgaatgttaatttttcccaggctgatgaggtaatagctgatttacga
gacaaacttcatcaattgtgtgaaatgctatttaatcaatctgtagctccctatgcacatcat
cctaaattaataagcacattagcgcttgcccgcgccacgctatacaaacatttacgggagtta
aaaccgcaaggagggccatcaaatggctga
>protein sequence of His6-Dual-monopartite NLS
SV40-FokI(Sharkey-ELD)-Cse1
[SEQ ID NO: 25]
MHHEIHHHPKKKRKVDPKKKRKVEDPKDMAQLVKSELEEKKSELRHKLKYVPHEYIELIEIAR
NPTQDRILEMKVMEFFMKVYGYRGEHLGGSRKPDGAIYTVGSPIDYGVIVDTKAYSGGYNLPI
GQADEMERYVEENQTRDKHLNPNEWWKVYPSSVTEFKFLFVSGHFKGNYKAQLTRLNHITNCN
GAVLSVEELLIGGEMIKAGTLTLEEVRRKFNNGEINFADPTNRAKGLEAVSVASMNLLIDNWI
PVRPRNGGKVQIINLQSLYCSRDQWRLSLPRDDMELAALALLVCIGQIIAPAKDDVEFRHRIM
NPLTEDEFQQLIAPWIDMFYLNHAEHPFMQTKGVKANDVTPMEKLLAGVSGATNCAFVNQPGQ
GEALCGGCTAIALFNQANQAPGFGGGFKSGLRGGTPVTTFVRGIDLRSTVLLNVLTLPRLQKQ
FPNESHTENQPTWIKPIKSNESIPASSIGFVRGLFWQPAHIELCDPIGIGKCSCCGQESNLRY
TGFLKEKFTFTVNGLWPHPHSPCLVTVKKGEVEEKFLAFTTSAPSWTQISRVVVDKIIQNENG
NRVAAVVNQFRNIAPQSPLELIMGGYRNNQASILERRHDVLMFNQGWQQYGNVINEIVTVGLG
YKTALRKALYTFAEGFKNKDFKGAGVSVHETAERHFYRQSELLIPDVLANVNFSQADEVIADL
RDKLHQLCEMLFNQSVAPYAHHPKLISTLALARATLYKHLRELKPQGGPSNG*

### DNA Cleavage Assay

The specificity and activity of the complexes was tested using an artificially constructed target plasmid as a substrate. This plasmid contains M13 and P7 binding sites on opposing strands such that both FokI domains face each other (see FIG. 11). The distance between the Cascade binding sites varies between 25 and 50 basepairs with 5 bp increments. As the binding sites of Cascade need to be flanked by any of four known PAM sequences (5′-protospacer-CTT/CAT/CTC/CCT-3′ this distance range gives sufficient flexibility to design such a pair for almost any given sequence.

The sequences of the target plasmids used are as follows. The number indicated the distance between the M13 and P7 target sites. Protospacers are shown in bold, PAMs underlined:

Sequences of the target plasmids. The number indicates the distance between the M13 and P7 target sites. (protospacers in bold, PAMs underlined)

>50 bp
[SEQ ID NO: 26]
gaattcACAACGGTGAGCAAGTCACTGTTGGCAAGCCAGGATCTGAACAA
TACCGTCTTGCTTTCGAGCGCTAGCTCTAGAACTAGTCCTCAGCCTAGGC
CTCGTTCCGAAGCTGTCTTTCGCTGCTGAGGGTGACGATCCCGCATAGGC
GGCCTTTAACTCggatcc
>45 bp
[SEQ ID NO: 27]
gaattcACAACGGTGAGCAAGTCACTGTTGGCAAGCCAGGATCTGAACAA
TACCGTCTTTTCGAGCGCTAGCTCTAGAACTAGTCCTCAGCCTAGGCCTC
GTTCAAGCTGTCTTTCGCTGCTGAGGGTGACGATCCCGCATAGGCGGCCT
TTAACTCggatcc
>40 bp
[SEQ ID NO: 28]
gaattcACAACGGTGAGCAAGTCACTGTTGGCAAGCCAGGATCTGAACAA
TACCGTCTTCGAGCGCTAGCTCTAGAACTAGTCCTCAGCCTAGGCCTCGA
AGCTGTCTTTCGCTGCTGAGGGTGACGATCCCGCATAGGCGGCCTTTAAC
TCggatcc
>35 bp
[SEQ ID NO: 29]
gaattcACAACGGTGAGCAAGTCACTGTTGGCAAGCCAGGATCTGAACAA
TACCGTCTTGCGCTAGCTCTAGAACTAGTCCTCAGCCTAGGCCTAAGCTG
TCTTTCGCTGCTGAGGGTGACGATCCCGCATAGGCGGCCTTTAACTCgga
tcc
>30 bp
[SEQ ID NO: 30]
gaattcACAACGGTGAGCAAGTCACTGTTGGCAAGCCAGGATCTGAACAA
TACCGTCTTGCTAGCTCTAGAACTAGTCCTCAGCCTAGGAAGCTGTCTTT
CGCTGCTGAGGGTGACGATCCCGCATAGGCGGCCTTTAACTCggatcc
>25 bp
[SEQ ID NO: 31]
gaattcACAACGGTGAGCAAGTCACTGTTGGCAAGCCAGGATCTGAACAA
TACCGTCTTCTCTAGAACTAGTCCTCAGCCTAGGAAGCTGTCTTTCGCTG
CTGAGGGTGACGATCCCGCATAGGCGGCCTTTAACTCggatcc

Cleavage of the target plasmids was analysed on agarose gels, where negatively supercoiled (nSC) plasmid can be distinguished from linearized- or nicked plasmid. The cleavage site of the Cascade^{KKR/ELD} pair in a target vector was determined by isolating linear cleavage products from an agarose gel and filling in the recessed 3′ ends left by FokI cleavage with the Klenow fragment of E. coli DNA polymerase to create blunt ends. The linear vector was self-ligated, transformed, amplified, isolated and sequenced. Filling in of recessed 3′ ends and re-ligation will lead to extra nucleotides in the sequence that represents the overhang left by FokI cleavage.

By aligning the sequence reads to the original sequence, the cleavage sites can be found on a clonal level and mapped. Below, the additional bases incorporated into the sequence after filling in recessed 3′ ends left by FokI cleavage are underlined:

FokI cleavage
5′ CTTGCGCTAGCTCTAGAA [symbol] CTAGTCCTCAGCCTAGGCCTAAG 3′
3′ GAACGCGATCGAGATCTTGATC AGGAGTCGGATCCGGATTC 5′
3′ fill in, ligation
5′ CTTGCGCTAGCTCTAGAACTAG - CTAGTCCTCAGCCTAGGCCTAAG 3′
3′ GAACGCGATCGAGATCTTGATC - GATCAGGAGTCGGATCCGGATTC 5′

Reading from top to bottom, the 5′-3′ sequences above are SEQ ID NOs: 32-35, respectively.

### Cleavage of a Target Locus in Human Cells

The human CCR5 gene encodes the C-C chemokine receptor type 5 protein, which serves as the receptor for the human immunodeficiency virus (HIV) on the surface of white blood cells. The CCR5 gene is targeted using a pair of Cascade^{KKR/ELD} nucleases in addition to an artificial GFP locus. A suitable binding site pair is selected on the coding region of CCR5. Two separate CRISPR arrays containing uniform spacers targeting each of the binding sites are constructed using DNA synthesis (Geneart).

The human CCR5 target gene selection and CRISPR designs used are as follows:

>Part of genomic human CCR5 sequence,
containing whole ORF (position 347-1446).
[SEQ ID NO: 36]
GGTGGAACAAGATGGATTATCAAGTGTCAAGTCCAATCTATGACATCAAT
TATTATACATCGGAGCCCTGCCAAAAAATCAATGTGAAGCAAATCGCAGC
CCGCCTCCTGCCTCCGCTCTACTCACTGGTGTTCATCTTTGGTTTTGTGG
GCAACATGCTGGTCATCCTCATCCTGATAAACTGCAAAAGGCTGAAGAGC
ATGACTGACATCTACCTGCTCAACCTGGCCATCTCTGACCTGTTTTTCCT
TCTTACTGTCCCCTTCTGGGCTCACTATGCTGCCGCCCAGTGGGACTTTG
GAAATACAATGTGTCAACTCTTGACAGGGCTCTATTTTATAGGCTTCTTC
TCTGGAATCTTCTTCATCATCCTCCTGACAATCGATAGGTACCTGGCTGT
CGTCCATGCTGTGTTTGCTTTAAAAGCCAGGACGGTCACCTTTGGGGTGG
TGACAAGTGTGATCACTTGGGTGGTGGCTGTGTTTGCGTCTCTCCCAGGA
ATCATCTTTACCAGATCTCAAAAAGAAGGTCTTCATTACACCTGCAGCTC
TCATTTTCCATACAGTCAGTATCAATTCTGGAAGAATTTCCAGACATTAA
AGATAGTCATCTTGGGGCTGGTCCTGCCGCTGCTTGTCATGGTCATCTGC
TACTCGGGAATCCTAAAAACTCTGCTTCGGTGTCGAAATGAGAAGAAGAG
GCACAGGGCTGTGAGGCTTATCTTCACCATCATGATTGTTTATTTTCTCT
TCTGGGCTCCCTACAACATTGTCCTTCTCCTGAACACCTTCCAGGAATTC
TTTGGCCTGAATAATTGCAGTAGCTCTAACAGGTTGGACCAAGCTATGCA
GGTGACAGAGACTCTTGGGATGACGCACTGCTGCATCAACCCCATCATCT
ATGCCTTTGTCGGGGAGAAGTTCAGAAACTACCTCTTAGTCTTCTTCCAA
AAGCACATTGCCAAACGCTTCTGCAAATGCTGTTCTATTTTCCAGCAAGA
GGCTCCCGAGCGAGCAAGCTCAGTTTACACCCGATCCACTGGGGAGCAGG
AAATATCTGTGGGCTTGTGACACGGACTCAAGTGGGCTGGTGACCCAGTC

Red1/2: chosen target sites (distance: 34 bp, PAM 5′-CTT-3′). “Red 1 is first appearing underlined sequence in the above. Red2 is the second underlined sequence.

>CRISPR array red1 (italics = spacers,
bold = repeats)
[SEQ ID NO: 37]
ccatggTAATACGACTCACTATAGGGAGAATTAGCTGATCTTTAATAATA
AGGAAATGTTACATTAAGGTTGGTGGGTTGTTTTTATGGGAAAAAATGCT
TTAAGAACAAATGTATACTTTTAGAGAGTTCCCCGCGCCAGCGGGGATAA
ACCGCAAACACAGCATGGACGACAGCCAGGTACCTAGAGTTCCCCGCGCC
AGCGGGGATAAACCGCAAACACAGCATGGACGACAGCCAGGTACCTAGAG
TTCCCCGCGCCAGCGGGGATAAACCGCAAACACAGCATGGACGACAGCCA
GGTACCTAGAGTTCCCCGCGCCAGCGGGGATAAACCGAAAACAAAAGGCT
CAGTCGGAAGACTGGGCCTTTTGTTTTAACCCCTTGGGGCCTCTAAACGG
GTCTTGAGGGGTTTTTTGggtacc
>CRISPR array red2 (italics: spacers,
bold: repeats)
[SEQ ID NO: 38]
ccatggTAATACGACTCACTATAGGGAGAATTAGCTGATCTTTAATAATA
AGGAAATGTTACATTAAGGTTGGTGGGTTGTTTTTATGGGAAAAAATGCT
TTAAGAACAAATGTATACTTTTAGAGAGTTCCCCGCGCCAGCGGGGATAA
ACCGTGTGATCACTTGGGTGGTGGCTGTGTTTGCGTGAGTTCCCCGCGCC
AGCGGGGATAAACCGTGTGATCACTTGGGTGGTGGCTGTGTTTGCGTGAG
TTCCCCGCGCCAGCGGGGATAAACCGTGTGATCACTTGGGTGGTGGCTGT
GTTTGCGTGAGTTCCCCGCGCCAGCGGGGATAAACCGAAAACAAAAGGCT
CAGTCGGAAGACTGGGCCTTTTGTTTTAACCCCTTGGGGCCTCTAAACGG
GTCTTGAGGGGTTTTTTGggtacc

### Delivery of Cascade^{KKR/ELD} Into the Nucleus of Human Cells

Cascade is very stable as a multi-subunit protein-RNA complex and is easily produced in mg quantities in E. coli. Transfection or micro-injection of the complex in its intact form as purified from E. coli is used as methods of delivery (see FIG. 12). As shown in FIG. 12, Cascade-FokI nucleases are purified from E. coli and encapsulated in protein transfection vesicles. These are then fused with the cell membrane of human HepG2 cells releasing the nucleases in the cytoplasm (step 2). NLS sequences are then be recognized by importin proteins, which facilitate nucleopore passage (step 3). Cascade^{KKR} (open rectangle) and Cascade^{ELD} (filled rectangle) will then find and cleave their target site (step 4.), inducing DNA repair pathways that will alter the target site leading to desired changes. Cascade^{KKR/ELD} nucleases need to act only once and require no permanent presence in the cell encoded on DNA.

To deliver Cascade into human cells, protein transfection reagents are used from various sources including Pierce, NEB, Fermentas and Clontech. These reagents have recently been developed for the delivery of antibodies, and are useful to transfect a broad range of human cell lines with efficiencies up to 90%. Human HepG2 cells are transfected. Also, other cell lines including CHO-K1, COS-7, HeLa, and non-embryonic stem cells, are transfected.

To import the Cascade^{KKR/ELD} nuclease pair into the nucleus, a tandem monopartite nuclear localisation signal (NLS) from the large T-antigen of simian virus 40 (SV40) is fused to the N-terminus of FokI. This ensures import of only intact Cascade^{ELD/KKR} into the nucleus. (The nuclear pore complex translocates RNA polymerases (550 kDa) and other large protein complexes). As a check prior to transformations, the nuclease activity of the Cascade^{KKR/ELD} nuclease pair is checked in vitro using purified complexes and CCR5 PCR amplicons to exclude transfecting non-productive Cascade^{KKR/ELD} nuclease pairs.

### Surveyor Assay

Transfected cells are cultivated and passaged for several days. The efficiency of in vivo target DNA cleavage is then assessed by using the Surveyor assay of Guschin, D.Y., et al (2010) Methods Mol. Biol., 649: 247-256. Briefly, PCR amplicons of the target DNA locus will be mixed 1:1 with PCR amplicons from untreated cells. These are heated and allowed to anneal, giving rise to mismatches at target sites that have been erroneously repaired by NHEJ. A mismatch nuclease is then used to cleave only mismatched DNA molecules, giving a maximum of 50% of cleavage when target DNA cleavage by Cascade^{KKR/ELD} is complete. This procedure was then followed up by sequencing of the target DNA amplicons of treated cells. The assay allows for rapid assessment and optimization of the delivery procedure.

### Production of Cascade-Nuclease Pairs

The Cascade-nuclease complexes were constructed as explained above. Affinity purification from E. coli using the StrepII-tagged Cse2 subunit yields a complex with the expected stoichiometry when compared to native Cascade. Referring to FIG. 13, this shows the stoichiometry of native Cascade (1), Cascade^{KKR} with P7 CrRNA and Cascade^{ELD} with M13 CrRNA 24 h after purification using only Streptactin. Bands in native Cascade (1) are from top to bottom: Cse1, Cas7, Cas5, Cas6e, Cse2. Cascade^{KKR/ELD} show the FokI-Cse1 fusion band and an additional band representing Cse1 with a small part of FokI as a result of proteolytic degradation.

Apart from an intact FokI-Cse1 fusion protein, we observed that a fraction of the FokI-Cse1-fusion protein is proteolytically cleaved, resulting in a Cse1 protein with only the linker and a small part of FokI attached to it (as confirmed by Mass Spectrometry, data not shown). In most protein isolations the fraction of degraded fusion protein is approximately 40%. The isolated protein is stably stored in the elution buffer (20 mM HEPES pH 7.5, 75 mM NaCl, 1 mM DTT, 4 mM desthiobiotin) with additional 0.1% Tween 20 and 50% glycerol at −20° C. Under these storage conditions, integrity and activity of the complex have been found stable for at least three weeks (data not shown).

### Introduction of a His6-Tag and NLS to the Cascade-Nuclease

The Cascade nuclease fusion design was modified to incorporate a Nucleolar Localization Signal (NLS) to enable transport into the nucleus of eukaryotic cells. For this a tandem monopartite NLS from the large T-antigen of Simian Virus SV40 (sequence:

PKKKRKVDPKKKRKV) was translationally fused to the N-terminus of the FokI-Cse1 fusion protein, directly preceded by a His6-tag at the N-terminus. The His₆-tag (sequence: MHHHHHH) allows for an additional Ni²⁺-resin affinity purification step after StrepII purification. This additional step ensures the isolation of only full-length Cascade-nuclease fusion complex, and increases the efficiency of cleavage by eliminating the binding of non-intact Cascade complexes to the target site forming an unproductive nuclease pair.

### In Vitro Cleavage Assay

Cascade^{KKR/ELD} activity and specificity was assayed in vitro as described above. FIG. 14A shows plasmids with distances between protospacers of 25-50 bp (5 bp increments, lanes 1-6) incubated with Cascade^{KKR/ELD} for 30 minutes at 37° C. Lane 10 contains the target plasmid in its three possible topologies: the lowest band represents the initial, negatively supercoiled (nSC) form of the plasmid, the middle band represents the linearized form (cleaved by XbaI), whilst the upper band represents the open circular (OC) form (after nicking with Nt.BbrCI). Lane 7 shows incubation of a plasmid with both binding sites removed (negative control). Therefore FIG. 14A shows a typical cleavage assay using various target plasmids in which the binding sites are separated by 25 to 50 base pairs in 5 bp increments (lanes 1 to 6). These plasmids with distances of 25-50 bp were incubated with Cascade^{KKR/ELD} carrying anti P7 and M13 crRNA respectively. A plasmid containing no binding sites served as a control (lane 7). The original plasmid exists in negatively supercoiled form (nSC, control lane 8), and nicked or linearized products are clearly distinguishable. Upon incubation a linear cleavage product is formed when binding sites were separated by 30, 35 and 40 base pairs (lanes 2, 3, 4). At 25, 45 and 50 base pairs distance (lanes 1, 5, 6), the target plasmid appeared to be incompletely cleaved leading to the nicked form (OC). These results show the best cleavage in plasmids with distances between 30 and 40 bp, giving sufficient flexibility when designing a crRNA pair for any given locus. Both shorter and longer distances result in increased nicking activity while creating less DSBs. There is very little activity on a plasmid where the two protospacers have been removed, showing target specificity (lane 7).

### Cleavage Conditions

To assess the optimal buffer conditions for cleavage assays, and to estimate whether activity of the complex is expected at physiological conditions, the following two buffers were selected: (1) NEB4 (New England Biolabs, 50 mM potassium acetate, 20 mM Tris-acetate, 10 mM magnesium acetate, 1 mM dithiothreitol, pH 7.9) and (2) Buffer O (Fermentas, 50 mM Tris-HCl, 10 mM MgCl₂, 100 mM NaCl, 0.1 mg/mL BSA, pH 7.5). Of the two, NEB4 is recommended for optimal activity of the commercial intact FokI enzyme. Buffer O was chosen from a quick screen to give good activity and specificity (data not shown). FIG. 14B shows incubation with different buffers and different incubation times. Lanes 1-4 have been incubated with Fermentas Buffer O (lane 1, 2 for 15 minutes, lane 3, 4 for 30 minutes), lanes 5, 6 have been incubated with NEB4 (30 minutes). Lanes 1, 3, 5 used the target plasmid with 35 bp spacing, lanes 2, 4, 6 used the non-target plasmid (no binding sites). Lanes 7, 8 have been incubated with only Cascade^{KKR} or Cascade^{ELD} respectively (buffer O). Lane 9 is the topology marker as in (A). Lane 10 and 11 show the target and non-target plasmid incubated without addition of Cascade. Therefore in FIG. 14B, activity was tested on the target plasmid with 35 base pairs distance (lane 1, 3, 5) and a non-target control plasmid (lane 2, 4, 6). There was a high amount of unspecific nicking and less cleavage in NEB4 (lane 5,6), whilst buffer O shows only activity in the target plasmid with a high amount of specific cleavage and little nicking (lane 1-4). The difference is likely caused by the NaCl concentration in buffer O, higher ionic strength weakens protein-protein interactions, leading to less nonspecific activity. Incubation of 15 or 30 minutes shows little difference in both target and non-target plasmid (lane 1,2 or 3,4 respectively). Addition of only one type of Cascade (P7^{KKR} or M13^{ELD}) does not result in cleavage activity (lane 7, 8) as expected. This experiment shows that specific Cascade nuclease activity by a designed pair occurs when the NaCl concentration is at least 100 mM, which is near the physiological saline concentration inside cells (137 mM NaCl). The Cascade nuclease pair is expected to be fully active in vivo, in eukaryotic cells, while displaying negligible off-target cleavage activity.

### Cleavage Site

The site of cleavage in the target plasmid with a spacing of 35 bp (pTarget35) was determined. FIG. 15 shows how sequencing reveals up- and downstream cleavage sites by Cascade^{KKR/ELD} in the target plasmid with 35 base pair spacing. In FIG. 15A) is shown the target region within pTarget35 with annotated potential cleavage sites. Parts of the protospacers are indicated in red and blue. B) The bar chart shows four different cleavage patterns and their relative abundance within sequenced clones. The blue bars represent the generated overhang, while the left and right border of each bar represents the left and right cleavage site (see B for annotation).

FIG. 15A shows the original sequence of pTarget35, with numbered cleavage sites from −7 to +7 where 0 lies in the middle between the two protospacers (indicated in red and blue). Seventeen clones were sequenced and these all show cleavage around position 0, creating varying overhangs between 3 and 5 bp (see FIG. 15B). Overhangs of 4 are most abundant (cumulatively 88%), while overhangs of 3 and 5 occur only once (6% each). The cleavage occurred exactly as expected with no clones showing off target cleavage.

### Cleaving a Target Locus in Human Cells.

Cascade^{KKR/ELD} nucleases were successfully modified to contain an N-terminal His6-tag followed by a dual mono-partite Nucleolar Localisation Signal. These modified Cascade nuclease fusion proteins were co-expressed with either one of two synthetically constructed CRISPR arrays, each targeting a binding site in the human CCR5 gene. First the activity of this new nuclease pair is validated in vitro by testing the activity on a plasmid containing this region of the CCR5 gene. The nuclease pair is transfected to a human cell line, e.g. HeLa cell line. Efficiency of target cleavage is assessed using the Surveyor assay as described above.

1-65. (canceled)

66. A Type I CRISPR composition comprising:
a first Type I CASCADE protein complex comprising a first Cse1 subunit protein, having an N-terminus and a C-terminus; a first Escherichia coli (E. coli) Cas3 mutant protein fused to the N-terminus of the first Cse1 subunit protein by a first linker polypeptide; and a first CRISPR-derived RNA (crRNA) molecule comprising a spacer sequence complementary to a first target nucleic acid, and
a second Type I CASCADE protein complex comprising a second Cse1 subunit protein, having an N-terminus and a C-terminus; a second E. coli Cas3 mutant protein fused to the N-terminus of the second Cse1 subunit protein by a second linker polypeptide; and a second CRISPR-derived RNA (crRNA) molecule comprising a spacer sequence complementary to a second target nucleic acid.

67. The composition of claim 66, wherein the first Type I CASCADE protein complex and the second Type I CASCADE protein complex form a heterodimer.

68. The Type I CRISPR composition of claim 66, wherein the first and the second Cas3 mutant proteins comprise one or more mutation in the helicase domain.

69. The Type I CRISPR composition of claim 68, wherein the first and the second E. coli Cas3 mutant proteins are selected form the group consisting of Cas3 K320N, Cas3 D452N, and Cas3 S483A/T485A.

70. The Type I CRISPR composition of claim 69, wherein the first and the second E. coli Cas3 mutant proteins are Cas3 D452N.

71. The Type I CRISPR composition of claim 66, wherein the first and the second linker polypeptides comprise a Streptomyces griseus linker polypeptide.

72. The Type I CRISPR composition of claim 66, wherein the first and the second Type I CASCADE protein complexes further comprise a Type I CRISPR associated subunit protein selected from the group consisting of a Cas7 subunit protein, a Cas5 subunit protein, and a Cas6 subunit protein.

73. The Type I CRISPR composition of claim 66, wherein the first and the second Type I CASCADE protein complexes further comprise a Type I CRISPR associated Cse2 subunit protein.

74. The Type I CRISPR composition of claim 56, wherein the first and the second Type I CASCADE protein complexes comprise the following stoichiometries: Cse1₁Cse2₂Cas7₆Cas5₁Cas6₁ or Cse1₁Cse2₂Cas7₆Cas5₁Cas6e₁.

75. The Type I CRISPR composition of claim 66, wherein each of the first and the second CRISPR-derived RNA (crRNA) molecules has a length between 35 and 75 nucleotides.

76. The Type I CRISPR composition of claim 75, wherein each of the first and the second CRISPR-derived RNA (crRNA) molecules has a length of 61 nucleotides.

77. The Type I CRISPR composition of claim 66, wherein each of the first and the second Type I CASCADE protein complexes further comprises a nuclear localization signal.

78. A method of cleaving a double-stranded target DNA sequence, the method comprising contacting the double-stranded target DNA sequence with the Type I CRISPR composition of claim 1, wherein the first strand of the double-stranded target DNA sequence comprises a first target nucleic acid and a first cleavage site, and the second strand of the double-stranded target DNA sequence comprises a second target nucleic acid and a second cleavage site, and
wherein the first Type I CASCADE protein complex binds the first target nucleic acid and cleaves the first cleavage site, and the second Type I CASCADE protein complex binds the second target nucleic acid and cleaves the second cleavage site.

79. The method of claim 78, wherein the double-stranded target DNA sequence is in genomic DNA.

80. A cell comprising the Type I CRISPR composition of claim 66.

81. The cell of claim 80, wherein the cell is a prokaryotic cell.

82. The cell of claim 80, wherein the cell is selected from the group consisting of a plant cell, a fungal cell, an animal cell, a yeast cell, an insect cell, a mammalian cell, and a human cell.


SEQUENCE LISTING (50 sequences)

SEQ ID NO: 1 - protein, 502 aa, Escherichia coli
MNLLIDNWIPVRPRNGGKVQIINLQSLYCSRDQWRLSLPRDDMELAALALLVCIGQIIAPAKDDVEFRHRIMNPLTEDEFQQLIAPWIDMFYLNHAEHPFMQTKGVKANDVTPMEKLLAGVSGATNCAFVNQPGQGEALCGGCTAIALFNQANQAPGFGGGFKSGLRGGTPVTTFVRGIDLRSTVLLNVLTLPRLQKQFPNESHTENQPTWIKPIKSNESIPASSIGFVRGLFWQPAHIELCDPIGIGKCSCCGQESNLRYTGFLKEKFTFTVNGLWPHPHSPCLVTVKKGEVEEKFLAFTTSAPSWTQISRVVVDKIIQNENGNRVAAVVNQFRNIAPQSPLELIMGGYRNNQASILERRHDVLMFNQGWQQYGNVINEIVTVGLGYKTALRKALYTFAEGFKNKDFKGAGVSVHETAERHFYRQSELLIPDVLANVNFSQADEVIADLRDKLHQLCEMLFNQSVAPYAHHPKLISTLALARATLYKHLRELKPQGGPSNG

SEQ ID NO: 2 - protein, 160 aa, Escherichia coli
MADEIDAMALYRAWQQLDNGSCAQIRRVSEPDELRDIPAFYRLVQPFGWENPRHQQALLRMVFCLSAGKNVIRHQDKKSEQTTGISLGRALANSGRINERRIFQLIRADRTADMVQLRRLLTHAEPVLDWPLMARMLTWWGKRERQQLLEDFVLTTNKNA

SEQ ID NO: 3 - protein, 363 aa, Escherichia coli
MSNFINIHVLISHSPSCLNRDDMNMQKDAIFGGKRRVRISSQSLKRAMRKSGYYAQNIGESSLRTIHLAQLRDVLRQKLGERFDQKIIDKTLALLSGKSVDEAEKISADAVTPWVVGEIAWFCEQVAKAEADNLDDKKLLKVLKEDIAAIRVNLQQGVDIALSGRMATSGMMTELGKVDGAMSIAHAITTHQVDSDIDWFTAVDDLQEQGSAHLGTQEFSSGVFYRYANINLAQLQENLGGASREQALEIATHVVHMLATEVPGAKQRTYAAFNPADMVMVNFSDMPLSMANAFEKAVKAKDGFLQPSIQAFNQYWDRVANGYGLNGAAAQFSLSDVDPITAQVKQMPTLEQLKSWVRNNGEA

SEQ ID NO: 4 - protein, 224 aa, Escherichia coli
MRSYLILRLAGPMQAWGQPTFEGTRPTGRFPTRSGLLGLLGACLGIQRDDTSSLQALSESVQFAVRCDELILDDRRVSVTGLRDYHTVLGAREDYRGLKSHETIQTWREYLCDASFTVALWLTPHATMVISELEKAVLKPRYTPYLGRRSCPLTHPLFLGTCQASDPQKALLNYEPVGGDIYSEESVTGHHLKFTARDEPMITLPRQFASREWYVIKGGMDVSQ

SEQ ID NO: 5 - protein, 199 aa, Escherichia coli
MYLSKVIIARAWSRDLYQLHQGLWHLFPNRPDAARDFLFHVEKRNTPEGCHVLLQSAQMPVSTAVATVIKTKQVEFQLQVGVPLYFRLRANPIKTILDNQKRLDSKGNIKRCRVPLIKEAEQIAWLQRKLGNAARVEDVHPISERPQYFSGDGKSGKIQTVCFEGVLTINDAPALIDLVQQGIGPAKSMGCGLLSLAPL

SEQ ID NO: 6 - DNA, 392 bp, synthetic construct
  misc_feature 1..392: note = Description of Artificial Sequence: Synthetic polynucleotide
  source 1..392: mol_type = other DNA
actggaaagcgggcagtgaaaggaaggcccatgaggccagttaattaagcggatcctggcggcggcagcggcggcggcagcgacaagcagaagaacggcatcaaggcgaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaagcggccgcggcgcgcctaggccttgacggccttccttcaattcgccctatagtgag

SEQ ID NO: 7 - DNA, 603 bp, synthetic construct
  misc_feature 1..603: note = Description of Artificial Sequence: Synthetic polynucleotide
  source 1..603: mol_type = other DNA
cactatagggcgaattggcggaaggccgtcaaggccgcatttaattaagcggccgcaggcggcggcagcggcggcggcagcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctcatctgcaccaccggcaagctgcccgtgccctggcccaccctcgtgaccaccctcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcacggcctaactcgagggcgcgccctgggcctcatgggccttccgctcactgcccgctttccag

SEQ ID NO: 8 - DNA, 679 bp, synthetic construct
  misc_feature 1..679: note = Description of Artificial Sequence: Synthetic polynucleotide
  source 1..679: mol_type = other DNA
cactatagggcgaattggcggaaggccgtcaaggccgcatgagctccatggaaacaaagaattagctgatctttaataataaggaaatgttacattaaggttggtgggttgtttttatgggaaaaaatgctttaagaacaaatgtatacttttagagagttccccgcgccagcggggataaaccgggccgattgaaggtccggtggatggcttaaaagagttccccgcgccagcggggataaaccgccgcaggtacagcaggtagcgcagatcatcaagagttccccgcgccagcggggataaaccgacttctctccgaaaagtcaggacgctgtggcagagttccccgcgccagcggggataaaccgcctacgcgctgaacgccagcggtgtggtgaatgagttccccgcgccagcggggataaaccggtgtggccatgcacgcctttaacggtgaactggagttccccgcgccagcggggataaaccgcacgaactcagccagaacgacaaacaaaaggcgagttccccgcgccagcggggataaaccggcaccagtacgcgccccacgctgacggtttctgagttccccgcgccagcggggataaaccgcagctcccattttcaaacccaggtaccctgggcctcatgggccttccgctcactgcccgctttccag

SEQ ID NO: 9 - DNA, 685 bp, synthetic construct
  misc_feature 1..685: note = Description of Artificial Sequence: Synthetic polynucleotide
  source 1..685: mol_type = other DNA
gagctcccgggctgacggtaatagaggcacctacaggctccggtaaaacggaaacagcgctggcctatgcttggaaacttattgatcaacaaattgcggatagtgttatttttgccctcccaacacaagctaccgcgaatgctatgcttacgagaatggaagcgagcgcgagccacttattttcatccccaaatcttattcttgctcatggcaattcacggtttaaccacctctttcaatcaataaaatcacgcgcgattactgaacaggggcaagaagaagcgtgggttcagtgttgtcagtggttgtcacaaagcaataagaaagtgtttcttgggcaaatcggcgtttgcacgattgatcaggtgttgatttcggtattgccagttaaacaccgctttatccgtggtttgggaattggtagatctgttttaattgttaatgaagttcatgcttacgacacctatatgaacggcttgctcgaggcagtgctcaaggctcaggctgatgtgggagggagtgttattcttctttccgcaaccctaccaatgaaacaaaaacagaagcttctggatacttatggtctgcatacagatccagtggaaaataactccgcatatccactcattaactggcgaggtgtgaatggtgcgcaacgttttgatctgctagcggatccggtacc

SEQ ID NO: 10 - DNA, 37 bp, synthetic construct
  misc_feature 1..37: note = Description of Artificial Sequence: Synthetic primer
  source 1..37: mol_type = other DNA
atagcgccatggaaccttttaaatatatatgccatta

SEQ ID NO: 11 - DNA, 41 bp, synthetic construct
  misc_feature 1..41: note = Description of Artificial Sequence: Synthetic primer
  source 1..41: mol_type = other DNA
acagtgggatccgctttgggatttgcagggatgactctggt

SEQ ID NO: 12 - DNA, 43 bp, synthetic construct
  misc_feature 1..43: note = Description of Artificial Sequence: Synthetic primer
  source 1..43: mol_type = other DNA
atagcgtcatgaatttgcttattgataactggattcctgtacg

SEQ ID NO: 13 - DNA, 44 bp, synthetic construct
  misc_feature 1..44: note = Description of Artificial Sequence: Synthetic primer
  source 1..44: mol_type = other DNA
acagtggcggccgcgccatttgatggccctccttgcggttttaa

SEQ ID NO: 14 - DNA, 45 bp, synthetic construct
  misc_feature 1..45: note = Description of Artificial Sequence: Synthetic primer
  source 1..45: mol_type = other DNA
cgtatatcaaactttccaatagcatgaagagcaatgaaaaataac

SEQ ID NO: 15 - DNA, 23 bp, synthetic construct
  misc_feature 1..23: note = Description of Artificial Sequence: Synthetic primer
  source 1..23: mol_type = other DNA
atgataccgcgagacccacgctc

SEQ ID NO: 16 - DNA, 24 bp, synthetic construct
  misc_feature 1..24: note = Description of Artificial Sequence: Synthetic primer
  source 1..24: mol_type = other DNA
cggataaagttgcaggaccacttc

SEQ ID NO: 17 - protein, 199 aa, Escherichia coli; identical to SEQ ID NO: 5

SEQ ID NO: 18 - DNA, 2154 bp, synthetic construct
  misc_feature 1..2154: note = Description of Artificial Sequence: Synthetic polynucleotide
  source 1..2154: mol_type = other DNA
atggctcaactggttaaaagcgaactggaagagaaaaaaagtgaactgcgccacaaactgaaatatgtgccgcatgaatatatcgagctgattgaaattgcacgtaatccgacccaggatcgtattctggaaatgaaagtgatggaattttttatgaaagtgtacggctatcgcggtgaacatctgggtggtagccgtaaaccggatggtgcaatttataccgttggtagcccgattgattatggtgttattgttgataccaaagcctatagcggtggttataatctgccgattggtcaggcagatgaaatggaacgttatgtggaagaaaatcagacccgtgataaacatctgaatccgaatgaatggtggaaagtttatccgagcagcgttaccgagtttaaattcctgtttgttagcggtcacttcaaaggcaactataaagcacagctgacccgtctgaatcatattaccaattgtaatggtgcagttctgagcgttgaagaactgctgattggtggtgaaatgattaaagcaggcaccctgaccctggaagaagttcgtcgcaaatttaacaatggcgaaatcaactttgcggatcccaccaaccgcgcgaaaggcctggaagcggtgagcgtggcgagcatgaatttgcttattgataactggattcctgtacgcccgcgaaacggggggaaagtccaaatcataaatctgcaatcgctatactgcagtagagatcagtggcgattaagtttgccccgtgacgatatggaactggccgctttagcactgctggtttgcattgggcaaattatcgccccggcaaaagatgacgttgaatttcgacatcgcataatgaatccgctcactgaagatgagtttcaacaactcatcgcgccgtggatagatatgttctaccttaatcacgcagaacatccctttatgcagaccaaaggtgtcaaagcaaatgatgtgactccaatggaaaaactgttggctggggtaagcggcgcgacgaattgtgcatttgtcaatcaaccggggcagggtgaagcattatgtggtggatgcactgcgattgcgttattcaaccaggcgaatcaggcaccaggttttggtggtggttttaaaagcggtttacgtggaggaacacctgtaacaacgttcgtacgtgggatcgatcttcgttcaacggtgttactcaatgtcctcacattacctcgtcttcaaaaacaatttcctaatgaatcacatacggaaaaccaacctacctggattaaacctatcaagtccaatgagtctatacctgcttcgtcaattgggtttgtccgtggtctattctggcaaccagcgcatattgaattatgcgatcccattgggattggtaaatgttcttgctgtggacaggaaagcaatttgcgttataccggttttcttaaggaaaaatttacctttacagttaatgggctatggccccatccgcattccccttgtctggtaacagtcaagaaaggggaggttgaggaaaaatttcttgctttcaccacctccgcaccatcatggacacaaatcagccgagttgtggtagataagattattcaaaatgaaaatggaaatcgcgtggcggcggttgtgaatcaattcagaaatattgcgccgcaaagtcctcttgaattgattatggggggatatcgtaataatcaagcatctattcttgaacggcgtcatgatgtgttgatgtttaatcaggggtggcaacaatacggcaatgtgataaacgaaatagtgactgttggtttgggatataaaacagccttacgcaaggcgttatatacctttgcagaagggtttaaaaataaagacttcaaaggggccggagtctctgttcatgagactgcagaaaggcatttctatcgacagagtgaattattaattcccgatgtactggcgaatgttaatttttcccaggctgatgaggtaatagctgatttacgagacaaacttcatcaattgtgtgaaatgctatttaatcaatctgtagctccctatgcacatcatcctaaattaataagcacattagcgcttgcccgcgccacgctatacaaacatttacgggagttaaaaccgcaaggagggccatcaaatggctga

SEQ ID NO: 19 - protein, 717 aa, synthetic construct
  REGION 1..717: note = Description of Artificial Sequence: Synthetic polypeptide
MAQLVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADEMERYVEENQTRDKHLNPNEWWKVYPSSVTEFKFLFVSGHFKGNYKAQLTRLNHITNCNGAVLSVEELLIGGEMIKAGTLTLEEVRRKFNNGEINFADPTNRAKGLEAVSVASMNLLIDNWIPVRPRNGGKVQIINLQSLYCSRDQWRLSLPRDDMELAALALLVCIGQIIAPAKDDVEFRHRIMNPLTEDEFQQLIAPWIDMFYLNHAEHPFMQTKGVKANDVTPMEKLLAGVSGATNCAFVNQPGQGEALCGGCTAIALFNQANQAPGFGGGFKSGLRGGTPVTTFVRGIDLRSTVLLNVLTLPRLQKQFPNESHTENQPTWIKPIKSNESIPASSIGFVRGLFWQPAHIELCDPIGIGKCSCCGQESNLRYTGFLKEKFTFTVNGLWPHPHSPCLVTVKKGEVEEKFLAFTTSAPSWTQISRVVVDKIIQNENGNRVAAVVNQFRNIAPQSPLELIMGGYRNNQASILERRHDVLMFNQGWQQYGNVINEIVTVGLGYKTALRKALYTFAEGFKNKDFKGAGVSVHETAERHFYRQSELLIPDVLANVNFSQADEVIADLRDKLHQLCEMLFNQSVAPYAHHPKLISTLALARATLYKHLRELKPQGGPSNG

SEQ ID NO: 20 - DNA, 2154 bp, synthetic construct
  misc_feature 1..2154: note = Description of Artificial Sequence: Synthetic polynucleotide
  source 1..2154: mol_type = other DNA
atggctcaactggttaaaagcgaactggaagagaaaaaaagtgaactgcgccacaaactgaaatatgtgccgcatgaatatatcgagctgattgaaattgcacgtaatccgacccaggatcgtattctggaaatgaaagtgatggaattttttatgaaagtgtacggctatcgcggtgaacatctgggtggtagccgtaaaccggatggtgcaatttataccgttggtagcccgattgattatggtgttattgttgataccaaagcctatagcggtggttataatctgccgattggtcaggcagatgaaatgcagcgttatgtgaaagaaaatcagacccgcaacaaacatattaacccgaatgaatggtggaaagtttatccgagcagcgttaccgagtttaaattcctgtttgttagcggtcacttcaaaggcaactataaagcacagctgacccgtctgaatcgtaaaaccaattgtaatggtgcagttctgagcgttgaagaactgctgattggtggtgaaatgattaaagcaggcaccctgaccctggaagaagttcgtcgcaaatttaacaatggcgaaatcaactttgcggatcccaccaaccgcgcgaaaggcctggaagcggtgagcgtggcgagcatgaatttgcttattgataactggattcctgtacgcccgcgaaacggggggaaagtccaaatcataaatctgcaatcgctatactgcagtagagatcagtggcgattaagtttgccccgtgacgatatggaactggccgctttagcactgctggtttgcattgggcaaattatcgccccggcaaaagatgacgttgaatttcgacatcgcataatgaatccgctcactgaagatgagtttcaacaactcatcgcgccgtggatagatatgttctaccttaatcacgcagaacatccctttatgcagaccaaaggtgtcaaagcaaatgatgtgactccaatggaaaaactgttggctggggtaagcggcgcgacgaattgtgcatttgtcaatcaaccggggcagggtgaagcattatgtggtggatgcactgcgattgcgttattcaaccaggcgaatcaggcaccaggttttggtggtggttttaaaagcggtttacgtggaggaacacctgtaacaacgttcgtacgtgggatcgatcttcgttcaacggtgttactcaatgtcctcacattacctcgtcttcaaaaacaatttcctaatgaatcacatacggaaaaccaacctacctggattaaacctatcaagtccaatgagtctatacctgcttcgtcaattgggtttgtccgtggtctattctggcaaccagcgcatattgaattatgcgatcccattgggattggtaaatgttcttgctgtggacaggaaagcaatttgcgttataccggttttcttaaggaaaaatttacctttacagttaatgggctatggccccatccgcattccccttgtctggtaacagtcaagaaaggggaggttgaggaaaaatttcttgctttcaccacctccgcaccatcatggacacaaatcagccgagttgtggtagataagattattcaaaatgaaaatggaaatcgcgtggcggcggttgtgaatcaattcagaaatattgcgccgcaaagtcctcttgaattgattatggggggatatcgtaataatcaagcatctattcttgaacggcgtcatgatgtgttgatgtttaatcaggggtggcaacaatacggcaatgtgataaacgaaatagtgactgttggtttgggatataaaacagccttacgcaaggcgttatatacctttgcagaagggtttaaaaataaagacttcaaaggggccggagtctctgttcatgagactgcagaaaggcatttctatcgacagagtgaattattaattcccgatgtactggcgaatgttaatttttcccaggctgatgaggtaatagctgatttacgagacaaacttcatcaattgtgtgaaatgctatttaatcaatctgtagctccctatgcacatcatcctaaattaataagcacattagcgcttgcccgcgccacgctatacaaacatttacgggagttaaaaccgcaaggagggccatcaaatggctga

SEQ ID NO: 21 - protein, 717 aa, synthetic construct
  REGION 1..717: note = Description of Artificial Sequence: Synthetic polypeptide
MAQLVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINFADPTNRAKGLEAVSVASMNLLIDNWIPVRPRNGGKVQIINLQSLYCSRDQWRLSLPRDDMELAALALLVCIGQIIAPAKDDVEFRHRIMNPLTEDEFQQLIAPWIDMFYLNHAEHPFMQTKGVKANDVTPMEKLLAGVSGATNCAFVNQPGQGEALCGGCTAIALFNQANQAPGFGGGFKSGLRGGTPVTTFVRGIDLRSTVLLNVLTLPRLQKQFPNESHTENQPTWIKPIKSNESIPASSIGFVRGLFWQPAHIELCDPIGIGKCSCCGQESNLRYTGFLKEKFTFTVNGLWPHPHSPCLVTVKKGEVEEKFLAFTTSAPSWTQISRVVVDKIIQNENGNRVAAVVNQFRNIAPQSPLELIMGGYRNNQASILERRHDVLMFNQGWQQYGNVINEIVTVGLGYKTALRKALYTFAEGFKNKDFKGAGVSVHETAERHFYRQSELLIPDVLANVNFSQADEVIADLRDKLHQLCEMLFNQSVAPYAHHPKLISTLALARATLYKHLRELKPQGGPSNG

SEQ ID NO: 22 - DNA, 2235 bp, synthetic construct
  misc_feature 1..2235: note = Description of Artificial Sequence: Synthetic polynucleotide
  source 1..2235: mol_type = other DNA
atgcatcaccatcatcaccacccgaaaaaaaagcgcaaagtggatccgaagaaaaaacgtaaagttgaagatccgaaagacatggctcaactggttaaaagcgaactggaagagaaaaaaagtgaactgcgccacaaactgaaatatgtgccgcatgaatatatcgagctgattgaaattgcacgtaatccgacccaggatcgtattctggaaatgaaagtgatggaattttttatgaaagtgtacggctatcgcggtgaacatctgggtggtagccgtaaaccggatggtgcaatttataccgttggtagcccgattgattatggtgttattgttgataccaaagcctatagcggtggttataatctgccgattggtcaggcagatgaaatgcagcgttatgtgaaagaaaatcagacccgcaacaaacatattaacccgaatgaatggtggaaagtttatccgagcagcgttaccgagtttaaattcctgtttgttagcggtcacttcaaaggcaactataaagcacagctgacccgtctgaatcgtaaaaccaattgtaatggtgcagttctgagcgttgaagaactgctgattggtggtgaaatgattaaagcaggcaccctgaccctggaagaagttcgtcgcaaatttaacaatggcgaaatcaactttgcggatcccaccaaccgcgcgaaaggcctggaagcggtgagcgtggcgagcatgaatttgcttattgataactggattcctgtacgcccgcgaaacggggggaaagtccaaatcataaatctgcaatcgctatactgcagtagagatcagtggcgattaagtttgccccgtgacgatatggaactggccgctttagcactgctggtttgcattgggcaaattatcgccccggcaaaagatgacgttgaatttcgacatcgcataatgaatccgctcactgaagatgagtttcaacaactcatcgcgccgtggatagatatgttctaccttaatcacgcagaacatccctttatgcagaccaaaggtgtcaaagcaaatgatgtgactccaatggaaaaactgttggctggggtaagcggcgcgacgaattgtgcatttgtcaatcaaccggggcagggtgaagcattatgtggtggatgcactgcgattgcgttattcaaccaggcgaatcaggcaccaggttttggtggtggttttaaaagcggtttacgtggaggaacacctgtaacaacgttcgtacgtgggatcgatcttcgttcaacggtgttactcaatgtcctcacattacctcgtcttcaaaaacaatttcctaatgaatcacatacggaaaaccaacctacctggattaaacctatcaagtccaatgagtctatacctgcttcgtcaattgggtttgtccgtggtctattctggcaaccagcgcatattgaattatgcgatcccattgggattggtaaatgttcttgctgtggacaggaaagcaatttgcgttataccggttttcttaaggaaaaatttacctttacagttaatgggctatggccccatccgcattccccttgtctggtaacagtcaagaaaggggaggttgaggaaaaatttcttgctttcaccacctccgcaccatcatggacacaaatcagccgagttgtggtagataagattattcaaaatgaaaatggaaatcgcgtggcggcggttgtgaatcaattcagaaatattgcgccgcaaagtcctcttgaattgattatggggggatatcgtaataatcaagcatctattcttgaacggcgtcatgatgtgttgatgtttaatcaggggtggcaacaatacggcaatgtgataaacgaaatagtgactgttggtttgggatataaaacagccttacgcaaggcgttatatacctttgcagaagggtttaaaaataaagacttcaaaggggccggagtctctgttcatgagactgcagaaaggcatttctatcgacagagtgaattattaattcccgatgtactggcgaatgttaatttttcccaggctgatgaggtaatagctgatttacgagacaaacttcatcaattgtgtgaaatgctatttaatcaatctgtagctccctatgcacatcatcctaaattaataagcacattagcgcttgcccgcgccacgctatacaaacatttacgggagttaaaaccgcaaggagggccatcaaatggctga

SEQ ID NO: 23 - protein, 744 aa, synthetic construct
  REGION 1..744: note = Description of Artificial Sequence: Synthetic polypeptide
MHHHHHHPKKKRKVDPKKKRKVEDPKDMAQLVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINFADPTNRAKGLEAVSVASMNLLIDNWIPVRPRNGGKVQIINLQSLYCSRDQWRLSLPRDDMELAALALLVCIGQIIAPAKDDVEFRHRIMNPLTEDEFQQLIAPWIDMFYLNHAEHPFMQTKGVKANDVTPMEKLLAGVSGATNCAFVNQPGQGEALCGGCTAIALFNQANQAPGFGGGFKSGLRGGTPVTTFVRGIDLRSTVLLNVLTLPRLQKQFPNESHTENQPTWIKPIKSNESIPASSIGFVRGLFWQPAHIELCDPIGIGKCSCCGQESNLRYTGFLKEKFTFTVNGLWPHPHSPCLVTVKKGEVEEKFLAFTTSAPSWTQISRVVVDKIIQNENGNRVAAVVNQFRNIAPQSPLELIMGGYRNNQASILERRHDVLMFNQGWQQYGNVINEIVTVGLGYKTALRKALYTFAEGFKNKDFKGAGVSVHETAERHFYRQSELLIPDVLANVNFSQADEVIADLRDKLHQLCEMLFNQSVAPYAHHPKLISTLALARATLYKHLRELKPQGGPSNG

SEQ ID NO: 24 - DNA, 2235 bp, synthetic construct
  misc_feature 1..2235: note = Description of Artificial Sequence: Synthetic polynucleotide
  source 1..2235: mol_type = other DNA
atgcatcaccatcatcaccacccgaaaaaaaagcgcaaagtggatccgaagaaaaaacgtaaagttgaagatccgaaagacatggctcaactggttaaaagcgaactggaagagaaaaaaagtgaactgcgccacaaactgaaatatgtgccgcatgaatatatcgagctgattgaaattgcacgtaatccgacccaggatcgtattctggaaatgaaagtgatggaattttttatgaaagtgtacggctatcgcggtgaacatctgggtggtagccgtaaaccggatggtgcaatttataccgttggtagcccgattgattatggtgttattgttgataccaaagcctatagcggtggttataatctgccgattggtcaggcagatgaaatggaacgttatgtggaagaaaatcagacccgtgataaacatctgaatccgaatgaatggtggaaagtttatccgagcagcgttaccgagtttaaattcctgtttgttagcggtcacttcaaaggcaactataaagcacagctgacccgtctgaatcatattaccaattgtaatggtgcagttctgagcgttgaagaactgctgattggtggtgaaatgattaaagcaggcaccctgaccctggaagaagttcgtcgcaaatttaacaatggcgaaatcaactttgcggatcccaccaaccgcgcgaaaggcctggaagcggtgagcgtggcgagcatgaatttgcttattgataactggattcctgtacgcccgcgaaacggggggaaagtccaaatcataaatctgcaatcgctatactgcagtagagatcagtggcgattaagtttgccccgtgacgatatggaactggccgctttagcactgctggtttgcattgggcaaattatcgccccggcaaaagatgacgttgaatttcgacatcgcataatgaatccgctcactgaagatgagtttcaacaactcatcgcgccgtggatagatatgttctaccttaatcacgcagaacatccctttatgcagaccaaaggtgtcaaagcaaatgatgtgactccaatggaaaaactgttggctggggtaagcggcgcgacgaattgtgcatttgtcaatcaaccggggcagggtgaagcattatgtggtggatgcactgcgattgcgttattcaaccaggcgaatcaggcaccaggttttggtggtggttttaaaagcggtttacgtggaggaacacctgtaacaacgttcgtacgtgggatcgatcttcgttcaacggtgttactcaatgtcctcacattacctcgtcttcaaaaacaatttcctaatgaatcacatacggaaaaccaacctacctggattaaacctatcaagtccaatgagtctatacctgcttcgtcaattgggtttgtccgtggtctattctggcaaccagcgcatattgaattatgcgatcccattgggattggtaaatgttcttgctgtggacaggaaagcaatttgcgttataccggttttcttaaggaaaaatttacctttacagttaatgggctatggccccatccgcattccccttgtctggtaacagtcaagaaaggggaggttgaggaaaaatttcttgctttcaccacctccgcaccatcatggacacaaatcagccgagttgtggtagataagattattcaaaatgaaaatggaaatcgcgtggcggcggttgtgaatcaattcagaaatattgcgccgcaaagtcctcttgaattgattatggggggatatcgtaataatcaagcatctattcttgaacggcgtcatgatgtgttgatgtttaatcaggggtggcaacaatacggcaatgtgataaacgaaatagtgactgttggtttgggatataaaacagccttacgcaaggcgttatatacctttgcagaagggtttaaaaataaagacttcaaaggggccggagtctctgttcatgagactgcagaaaggcatttctatcgacagagtgaattattaattcccgatgtactggcgaatgttaatttttcccaggctgatgaggtaatagctgatttacgagacaaacttcatcaattgtgtgaaatgctatttaatcaatctgtagctccctatgcacatcatcctaaattaataagcacattagcgcttgcccgcgccacgctatacaaacatttacgggagttaaaaccgcaaggagggccatcaaatggctga

SEQ ID NO: 25 - protein, 744 aa, synthetic construct
  REGION 1..744: note = Description of Artificial Sequence: Synthetic polypeptide
MHHHHHHPKKKRKVDPKKKRKVEDPKDMAQLVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADEMERYVEENQTRDKHLNPNEWWKVYPSSVTEFKFLFVSGHFKGNYKAQLTRLNHITNCNGAVLSVEELLIGGEMIKAGTLTLEEVRRKFNNGEINFADPTNRAKGLEAVSVASMNLLIDNWIPVRPRNGGKVQIINLQSLYCSRDQWRLSLPRDDMELAALALLVCIGQIIAPAKDDVEFRHRIMNPLTEDEFQQLIAPWIDMFYLNHAEHPFMQTKGVKANDVTPMEKLLAGVSGATNCAFVNQPGQGEALCGGCTAIALFNQANQAPGFGGGFKSGLRGGTPVTTFVRGIDLRSTVLLNVLTLPRLQKQFPNESHTENQPTWIKPIKSNESIPASSIGFVRGLFWQPAHIELCDPIGIGKCSCCGQESNLRYTGFLKEKFTFTVNGLWPHPHSPCLVTVKKGEVEEKFLAFTTSAPSWTQISRVVVDKIIQNENGNRVAAVVNQFRNIAPQSPLELIMGGYRNNQASILERRHDVLMFNQGWQQYGNVINEIVTVGLGYKTALRKALYTFAEGFKNKDFKGAGVSVHETAERHFYRQSELLIPDVLANVNFSQADEVIADLRDKLHQLCEMLFNQSVAPYAHHPKLISTLALARATLYKHLRELKPQGGPSNG

SEQ ID NO: 26 - DNA, 168 bp, synthetic construct
  misc_feature 1..168: note = Description of Artificial Sequence: Synthetic polynucleotide
  source 1..168: mol_type = other DNA
gaattcacaacggtgagcaagtcactgttggcaagccaggatctgaacaataccgtcttgctttcgagcgctagctctagaactagtcctcagcctaggcctcgttccgaagctgtctttcgctgctgagggtgacgatcccgcataggcggcctttaactcggatcc

SEQ ID NO: 27 - DNA, 163 bp, synthetic construct
  misc_feature 1..163: note = Description of Artificial Sequence: Synthetic polynucleotide
  source 1..163: mol_type = other DNA
gaattcacaacggtgagcaagtcactgttggcaagccaggatctgaacaataccgtcttttcgagcgctagctctagaactagtcctcagcctaggcctcgttcaagctgtctttcgctgctgagggtgacgatcccgcataggcggcctttaactcggatcc

SEQ ID NO: 28 - DNA, 158 bp, synthetic construct
  misc_feature 1..158: note = Description of Artificial Sequence: Synthetic polynucleotide
  source 1..158: mol_type = other DNA
gaattcacaacggtgagcaagtcactgttggcaagccaggatctgaacaataccgtcttcgagcgctagctctagaactagtcctcagcctaggcctcgaagctgtctttcgctgctgagggtgacgatcccgcataggcggcctttaactcggatcc

SEQ ID NO: 29 - DNA, 153 bp, synthetic construct
  misc_feature 1..153: note = Description of Artificial Sequence: Synthetic polynucleotide
  source 1..153: mol_type = other DNA
gaattcacaacggtgagcaagtcactgttggcaagccaggatctgaacaataccgtcttgcgctagctctagaactagtcctcagcctaggcctaagctgtctttcgctgctgagggtgacgatcccgcataggcggcctttaactcggatcc

SEQ ID NO: 30 - DNA, 148 bp, synthetic construct
  misc_feature 1..148: note = Description of Artificial Sequence: Synthetic polynucleotide
  source 1..148: mol_type = other DNA
gaattcacaacggtgagcaagtcactgttggcaagccaggatctgaacaataccgtcttgctagctctagaactagtcctcagcctaggaagctgtctttcgctgctgagggtgacgatcccgcataggcggcctttaactcggatcc

SEQ ID NO: 31 - DNA, 143 bp, synthetic construct
  misc_feature 1..143: note = Description of Artificial Sequence: Synthetic polynucleotide
  source 1..143: mol_type = other DNA
gaattcacaacggtgagcaagtcactgttggcaagccaggatctgaacaataccgtcttctctagaactagtcctcagcctaggaagctgtctttcgctgctgagggtgacgatcccgcataggcggcctttaactcggatcc

SEQ ID NO: 32 - DNA, 41 bp, synthetic construct
  misc_feature 1..41: note = Description of Artificial Sequence: Synthetic oligonucleotide
  source 1..41: mol_type = other DNA
cttgcgctagctctagaactagtcctcagcctaggcctaag

SEQ ID NO: 33 - DNA, 41 bp, synthetic construct
  misc_feature 1..41: note = Description of Artificial Sequence: Synthetic oligonucleotide
  source 1..41: mol_type = other DNA
cttaggcctaggctgaggactagttctagagctagcgcaag

SEQ ID NO: 34 - DNA, 45 bp, synthetic construct
  misc_feature 1..45: note = Description of Artificial Sequence: Synthetic oligonucleotide
  source 1..45: mol_type = other DNA
cttgcgctagctctagaactagctagtcctcagcctaggcctaag

SEQ ID NO: 35 - DNA, 45 bp, synthetic construct
  misc_feature 1..45: note = Description of Artificial Sequence: Synthetic oligonucleotide
  source 1..45: mol_type = other DNA
cttaggcctaggctgaggactagctagttctagagctagcgcaag

SEQ ID NO: 36 - DNA, 1100 bp, Homo sapiens
  source 1..1100: mol_type = genomic DNA
ggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggcttgtgacacggactcaagtgggctggtgacccagtc

SEQ ID NO: 37 - DNA, 424 bp, synthetic construct
  misc_feature 1..424: note = Description of Artificial Sequence: Synthetic polynucleotide
  source 1..424: mol_type = other DNA
ccatggtaatacgactcactatagggagaattagctgatctttaataataaggaaatgttacattaaggttggtgggttgtttttatgggaaaaaatgctttaagaacaaatgtatacttttagagagttccccgcgccagcggggataaaccgcaaacacagcatggacgacagccaggtacctagagttccccgcgccagcggggataaaccgcaaacacagcatggacgacagccaggtacctagagttccccgcgccagcggggataaaccgcaaacacagcatggacgacagccaggtacctagagttccccgcgccagcggggataaaccgaaaacaaaaggctcagtcggaagactgggccttttgttttaaccccttggggcctctaaacgggtcttgaggggttttttgggtacc

SEQ ID NO: 38 - DNA, 424 bp, synthetic construct
  misc_feature 1..424: note = Description of Artificial Sequence: Synthetic polynucleotide
  source 1..424: mol_type = other DNA
ccatggtaatacgactcactatagggagaattagctgatctttaataataaggaaatgttacattaaggttggtgggttgtttttatgggaaaaaatgctttaagaacaaatgtatacttttagagagttccccgcgccagcggggataaaccgtgtgatcacttgggtggtggctgtgtttgcgtgagttccccgcgccagcggggataaaccgtgtgatcacttgggtggtggctgtgtttgcgtgagttccccgcgccagcggggataaaccgtgtgatcacttgggtggtggctgtgtttgcgtgagttccccgcgccagcggggataaaccgaaaacaaaaggctcagtcggaagactgggccttttgttttaaccccttggggcctctaaacgggtcttgaggggttttttgggtacc

SEQ ID NO: 39 - DNA, 43 bp, synthetic construct
  misc_feature 1..43: note = Description of Artificial Sequence: Synthetic oligonucleotide
  source 1..43: mol_type = other DNA
aaggatgccagtgataagtggaatgccatgtgggctgtcaaaa

SEQ ID NO: 40 - DNA, 43 bp, synthetic construct
  misc_feature 1..43: note = Description of Artificial Sequence: Synthetic oligonucleotide
  source 1..43: mol_type = other DNA
aaggatgcgagtgataagtggaatgccatgtgggctgtcaaaa

SEQ ID NO: 41 - DNA, 19 bp, synthetic construct
  misc_feature 1..19: note = Description of Artificial Sequence: Synthetic oligonucleotide
  source 1..19: mol_type = other DNA
gccatgtgggctgtcaaaa

SEQ ID NO: 42 - DNA, 23 bp, synthetic construct
  misc_feature 1..23: note = Description of Artificial Sequence: Synthetic oligonucleotide
  source 1..23: mol_type = other DNA
gaatgccatgtgggctgtcaaaa

SEQ ID NO: 43 - protein, 1604 aa, Streptomyces sp.
MPDQLNAPTPLGDRLTGAVRTVWAKHDRDTGKWLPLWRHMTDSAAVAGLLWDHWLPRNIKDLIAEPLPGGVADARSLCVWLAGTHDIGKATPAFACQVDELAGVMTAAGLDMRTSKQLGEDRRMAPHGLAGQVLLQEWLEERRGWTHRASAQFAVVAGGHHGVPPDHMQLHNLDAHPELLRTQGLAEAQWRAVQDELLDACALVFGVEERLDAWRTVKLPQTVQVLLTATVIVSDWIASNPDLFPYFPEEHPREEAERVAAAWQGLLLPAPWEPEEPSAPAAEFYASRFALPPGAVVRPVQEQALAMARDMERPGMLIIEAPMGEGKTEAALAVAEVFAARSGAGGCYVALPTMATSNAMFPRLLRWLDRLPRADVSGGRDHEQRSVLLAHAKSALQEDYATLMRESHRTIAAVDAYGDDSRPRKGRPAADGVRRKAPAELVAHQWLRGRKKGLLASFAVGTIDQLLMAGLKSRHLALRHLAMAGKVVVIDEVHAYDTYMNAYLDRVLAWLGEYRVPVVVLSATLPARRRGELAAAYTGEDAQALTEATGYPLLTAVVPGREAVQFVAAASGRGSDVLLEKLDDDDEALADRLDTDLADGGCALVVRNTVDRVMDTASVLRERFGADHVTVAHARFVDLDRARKDSELLARFGPPDPDGGSPQRPRNAHIVVASQVAEQSLDVDFDLLVSDLCPVDLLLQRMGRLHRHPRGRDQERRPARLRQARCLVTGVGWDTSPAPEADEGSRAIYGAYSLLRSLAVLAPHLGTAGAAGHPLRLPEDISPLVRRAYGEEDPCPPEWEPVLAPARDKYRTARERQSQKAEVFRLDEVRKAGRPLIGWIDAGVGDADDTPVGRAQVRDTKEGLEVLVVRRRADGSLCTLPWLDKGRGGLELPVDAVPSALAARAVAASGLRLPYHFTSSPQTLDRTLAELEELYVPAWQEKESHWIAGELILALDEEGRAALAGQQLVYNPEEGLLVASADANTEATSGRVMDGKPSSAGDGKPGHAADGNRARTTVGQSPADRQTHQPPEGERHPVPPSAAPPPARPSFDLTSRPWLPVLLKDGSERELSLPEVFDQARDIRRLVGDLPTQDFALTRMLLALLYDALSEPGGDMAPADTDAWEELWLSQSAYAAPVAAYLHRYRERFDLLHPESPFFQTPGLRTAKNEVFSLNRLVADVPNGDPFFSMRRPGVDRLGFAEAARWLVHAQAYDTSGIKTGAVGDPRVKAGKGYPQGPAWAGNLGGVLLEGDNLHETLLLNLIAGDTPGVHAAEVDRPAWRAEPSGPAPAPDLGLRPYGLRDLYTWQSRRIRLHHDADGVHGVVLAYGDSLEPHNRHGHEPMTSWRRSPTQEKKRQENLVYLPREHDPSRLAWRGMDGLLAGRETGSAQGPDGADRLAPKVVQWAAQLTTEGLLPRGYLIRTRVIGARYGTQQSVIDEVVDDGVLMPAVLLHEADRRYGDKAVDALHDAEKAVGALAQLAADLALAVGTDPEPGRNTARDLGFGTLDTHYRRWLRELGGTSDPEEHRDRWKQEVRRLVAELGERLLDGAGPAAWEGRLVETGKGTRWLNDAAAELRFRTRLREFLTTAPDTPTSPRPAPVESPA

SEQ ID NO: 44 - protein, 1559 aa, Streptomyces griseus
MSNTPMSRDHPESLSAYARLSPVSRTAWGKHDRQTEQWLPLWRHMADSAAVAERLWDQWVPDNVKALIADAFPQGAQDARRVAVFLACVHDIGKATPAFACQVDGLADRMRAAGLSMPYLKQFGLDRRMAPHGLAGQLLLQEWLAERFGWSERASGQFAVVAGGHHGTPPDHQHIHDLGLRPHLLRTAGESQDTWRSVQDELMDACAVRAGVGGRFGAWRSVRLPQPVQVVLTAIVIVSDWIASSSELFPYDPASWSPVGPEGEGRRLTAAWGGLDLPGPWRADQPDCTAAELFGKRFDLPEGAGVRPVQEEAVRVAQELPGPGLLIIEAPMGEGKTEAAFAAAEILAARTGAGGCLVALPTRATGDAMFPRLLRWLERLPSDGPRSVVLAHAKAALNEVWAGMTKADRRKITAVDLDSQVEDVSSAGGARRANPASLHAHQWLRGRKKALLSSFAVGTVDQVLFAGLKSRHLALRHLAVAGKVVIVDEVHAYDAYMSAYLDRVLEWLAAYRVPVVMLSATLPAHRRRELAAAYAGEETPELADALALPDDAYPLITAVAPGGLVLTARPEPASGRRTEVVLERLGDGPALLAARLDEELRDGGCALVVRNTVDRVLEAAEHLRAHFGAEAVTVAHSRFVAADRARNDTVLRERFGPGGDRPAGPHIVVASQVVEQSLDIDFDLLVTDLAPVDLVLQRMGRLHRHPRTRPPRLSRARCLITGVEDWHAERPVPVRGSLAVYQGPHTLLRALAVLGPHLDGVPLVLPDHISPLVQAAYDERPVGPAHWAPVLDEARRQYLTRLAEKRERADVFRLGPVRRPGRPLFGWLDGNAGDADDSRTGRAQVRDSEESLEVLVVQRRADGRLTTVSWLDGGRGGLDLPEHAPPPPRAAEVVAACALTLPRSLTHPGVIDRTIAELERFVVPAWQVKECPWLAGELLLVLDEDCQTRLSGLEVHYSTDQGLRVGSVGTRSTNRAKGLEAVSVASFDLVSRPWLPVQYEDGATGELSLREVFARAGEVRRLVGDLPTQELALLRLLLAILYDAYDEAPGRSGGAPAQLEDWEALWDEPDSFAVVAGYLDRHRDRFDLLHPERPFFQVAGLHTQKHEVASLNRIVADVPNGEAFFSMRRPGVHRLGLAEAARWLVHTHAYDASGIKSGMEGDARVKGGKVYPQGVGWVGGLGGVFAEGASLRETLLLNLIPTDEDILTSEPKADLPVWRRETPPGPGVVEGDPSAPRPAGPRDLYTWQSRRLLLHTEGSDAIGVVLGYGDPLSPANRQKTEPMTGWRRSPAQEKKLGRPLVYLPRQHDPGRAAWRGLASLLYPQGEDGDTTGRGTDRSRPAGIVRWLALLSTEGVLPKGSLIRTRLVGAVYGTQQSVVDDVVDDSIALPVVLLHQDRRLHGAVAVDAVADAERAVSALGHLAGNLARASGSEAGPATATARDQGFGALDGPYRRWLVDLAEDTDLERARAAWRDTVRLVVLGIGRELLDAAGRAAAEGRVIELPGVGKRWIDSSRADLWFRTRINRVLPRPLPEAHAPTADIHAGHAVRADEALSEETV

SEQ ID NO: 45 - protein, 1540 aa, Catenulispora acidiphila
MFNVGSTRCWGDGGLRNAAEDLSAATRSAWAKSDPDSGQSLSLIRHLADSAAIAEHLWDQWLPDHVKSLIAEGLPEGLVDGRTLAVWLAGTHDIGKLTPAFACQCEPLAQAMRECGLDMPTRTQFGDDRRVAPHGLAGQVLLREWLMERHGWSGRSADAFTVIAGGHHGVPPSYSQLHDLDAYPELLRTPGASEGIWKSSQHELLDACAVMTGASSRLAHWRGLRLSQQAQVLLTGLVIVADWIASNTDLFPYPALGTGEAAIDPGKRVELAWRGLELPAPWAPKYLMPGMQGLLASRFGLPADAQLRPVQQMAVQLASANAAPGLLVIEAPMGEGKTEAALLAAEILAARSGAGGVFLALPTQATSNAMFARVVNWLRQVPREGVASVHLAHGKAALDDAFASFLRAAPRLTSIDADGYAGEANVRRDRRAGSADMVAHQWLRGRKKGILSPFVVGTIDQLLFTGLKSRHLALRHLAVAGKVVVIDEVHAYDAYMSVYLERVLSWLGAYRVPVVLLSATLPADRRQALVEAYGGITSEALRDAREAYPVLTAVTIGAPAQAVGTEPAEGRRVDVNVEAFDDDLGRLADRLEAELVDGGCALIIRNTVGRVLQTAQQLRERFGAGQVTVAHSRFIDLDRARKDADLLARFGHDGARPRRHIVVASQVAEQSLDIDFDLLVTDLAPIDLVLQRMGRVHRHHRGGPEQSERPPSLRTARCLVTGVDWAGIPSAPIAGSVAVYGLHPLLRSLAVLQPYLTGSALTLPGDINPLVQCAYAQSFVAPTGWGEAMDAAQAEHMAHIVQQREGAMAFCLDEVRGPGRSLIGWIDGGVGDADDTRAGRAQVRDSPETIEVLVVQRGSDGVLRTLPWLDRGRGGLELPTEAVPPPRAARAAAASALRLPGLFAKPWMFDRVLRELEREYHEAWQAKESSWLQGELLLVLDEECRTVLAGYELSYNPDDGLEMVMPGEPHAAVVRDKEASDDKTASFDLTSAPWLPVLYADGMQGVLSLRDVFAQSNLIRRLVGDLPTQDFALLRLLLAVLYDAVDGPRDGQDWEDLWTSDDPFAAVPAYLDSHRERFDLLHPATPFYQVPGLQTAKGEVGPLNKIVADVPDGDPFLTMRMPGVEQLSFAEAARWLVHTQAFDTSGIKSGVVGDPKAVNGKRYPQGVAWLGNLGGVFAEGDTLRQTLLLNLIPADTTNLQVTSAQDVPAWRGTNGRAGSDHADAEPRVPAGLRDLYTWQSRRIRLEYDTRGVTGAVLTYGDELTAHNKHGVEPMTGWRRSKPQEKKLGLSTVYMPQQHDPTRAAWRGIESLLAGSAGSGSSQTGEPASHYRPKIVDWLGELAHHGNLPSRGLIRVRTSGAVYGTQQSIIDEVVSDELTMAVVLLHEDDPRFGKAAVTAVKDADSAVAALGDLASDLARAAGLDPEPERVTARDRAFGALDGPYRRWLLDLGNSTDPAAMRAVWQGRVYDIIAVQGQMLLDSAGSAAAQGRMVKTTRGERWMDDSLADLYFKGRIAKALSSRLGKKPTDPGEPVGIQEDPA

SEQ ID NO: 46 - protein, 1407 aa, synthetic construct
  REGION 1..1407: note = Description of Artificial Sequence: Synthetic polypeptide
MEPFKYICHYWGKSSKSLTKGNDIHLLIYHCLDVAAVADCWWDQSVVLQNTFCRNEMLSKQRVKAWLLFFIALHDIGKFDIRFQYKSAESWLKLNPATPSLNGPSTQMCRKFNHGAAGLYWFNQDSLSEQSLGDFFSFFDAAPHPYESWFPWVEAVTGHHGFILHSQDQDKSRWEMPASLASYAAQDKQAREEWISVLEALFLTPAGLSINDIPPDCSSLLAGFCSLADWLGSWTTTNTFLFNEDAPSDINALRTYFQDRQQDASRVLELSGLVSNKRCYEGVHALLDNGYQPRQLQVLVDALPVAPGLTVIEAPTGSGKTETALAYAWKLIDQQIADSVIFALPTQATANAMLTRMEASASHLFSSPNLILAHGNSRFNHLFQSIKSRAITEQGQEEAWVQCCQWLSQSNKKVFLGQIGVCTIDQVLISVLPVKHRFIRGLGIGRSVLIVDEVHAYDTYMNGLLEAVLKAQADVGGSVILLSATLPMKQKQKLLDTYGLHTDPVENNSAYPLINWRGVNGAQRFDLLAHPEQLPPRFSIQPEPICLADMLPDLTMLERMIAAANAGAQVCLICNLVDVAQVCYQRLKELNNTQVDIDLFHARFTLNDRREKENRVISNFGKNGKRNVGRILVATQVVEQSLDVDFDWLITQHCPADLLFQRLGRLHRHHRKYRPAGFEIPVATILLPDGEGYGRHEHIYSNVRVMWRTQQHIEELNGASLFFPDAYRQWLDSIYDDAEMDEPEWVGNGMDKFESAECEKRFKARKVLQWAEEYSLQDNDETILAVTRDGEMSLPLLPYVQTSSGKQLLDGQVYEDLSHEQQYEALALNRVNVPFTWKRSFSEVVDEDGLLWLEGKQNLDGWVWQGNSIVITYTGDEGMTRVIPANPKGDPTNRAKGLEAVSVASMNLLIDNWIPVRPRNGGKVQIINLQSLYCSRDQWRLSLPRDDMELAALALLVCIGQIIAPAKDDVEFRHRIMNPLTEDEFQQLIAPWIDMFYLNHAEHPFMQTKGVKANDVTPMEKLLAGVSGATNCAFVNQPGQGEALCGGCTAIALFNQANQAPGFGGGFKSGLRGGTPVTTFVRGIDLRSTVLLNVLTLPRLQKQFPNESHTENQPTWIKPIKSNESIPASSIGFVRGLFWQPAHIELCDPIGIGKCSCCGQESNLRYTGFLKEKFTFTVNGLWPHPHSPCLVTVKKGEVEEKFLAFTTSAPSWTQISRVVVDKIIQNENGNRVAAVVNQFRNIAPQSPLELIMGGYRNNQASILERRHDVLMFNQGWQQYGNVINEIVTVGLGYKTALRKALYTFAEGFKNKDFKGAGVSVHETAERHFYRQSELLIPDVLANVNFSQADEVIADLRDKLHQLCEMLFNQSVAPYAHHPKLISTLALARATLYKHLRELKPQGGPSNG

SEQ ID NO: 47 - DNA, 49 bp, synthetic construct
  misc_feature 1..49: note = Description of Artificial Sequence: Synthetic oligonucleotide
  source 1..49: mol_type = other DNA
ccgtcttgcgctagctctagaactagtcctcagcctaggcctaagctgt

SEQ ID NO: 48 - protein, 6 aa, synthetic construct
  REGION 1..6: note = Description of Artificial Sequence: Synthetic 6xHis tag
HHHHHH

SEQ ID NO: 49 - protein, 15 aa, unidentified
  REGION 1..15: note = Simian virus 40
PKKKRKVDPKKKRKV

SEQ ID NO: 50 - protein, 7 aa, synthetic construct
  REGION 1..7: note = Description of Artificial Sequence: Synthetic peptide
MHHHHHH